(19) United States
(12) Patent Application Publication
(10) Pub. No.: US 2019/0091173 A1
(43) Pub. Date: Mar. 28, 2019

(54) METHOD OF INCREASING TIME BETWEEN CONVULSIVE SEIZURES

(71) Applicant: ZOGENIX INTERNATIONAL LIMITED, Berkshire (GB)

(72) Inventors: Gail FARFEL, Franklin Lakes, NJ (US); Michael LOCK, Emeryville, CA (US); Brooks M. BOYD, Berkeley, CA (US)

(73) Assignee: ZOGENIX INTERNATIONAL LIMITED, Berkshire (GB)

(21) Appl. No.: 16/139,617

(22) Filed: Sep. 24, 2018

Related U.S. Application Data
(60) Provisional application No. 62/563,255, filed on Sep. 26, 2017.
(60) Provisional application No. 62/564,225, filed on Sep. 27, 2017.
(60) Provisional application No. 62/579,450, filed on Oct. 31, 2017.
(60) Provisional application No. 62/593,029, filed on Nov. 30, 2017.
(60) Provisional application No. 62/627,329, filed on Feb. 7, 2018.
(60) Provisional application No. 62/669,833, filed on May 10, 2018.
(60) Provisional application No. 62/696,801, filed on Jul. 11, 2018.

(51) Int. Cl. A61K 31/137; A61P 25/08

(52) U.S. Cl. CPC A61K 31/137; A61K 31/19; A61K 2300/00; A61P 25/08

(57) ABSTRACT

A method of increasing an average time between seizures in a human patient diagnosed with Dravet syndrome, comprising administering to the patient a therapeutically effective dose of fenfluramine or a pharmaceutically acceptable salt, base, acid or amine thereof, and repeating the administering over a period of days until the patient exhibits an increase from baseline in average time between convulsive seizures of 6 hours, days weeks or more.

### FIELD OF THE INVENTION

A method of treating patients with Dravet syndrome is described whereby the patient is repeatedly treated with fenfluramine and the treatment continued to obtain a desired end point not previously recognized.

### BACKGROUND OF THE INVENTION

This invention relates to the treatment of Dravet syndrome using an amphetamine derivative, specifically fenfluramine.

Fenfluramine, i.e. 3-trifluoromethyl-N-ethylamphetamine is an amphetamine derivative having the structure:

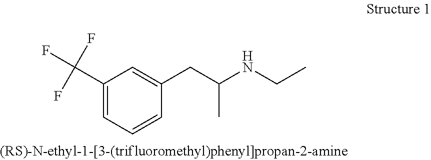 [SMILES: CCNC(C)Cc1cccc(C(F)(F)F)c1]

Fenfluramine was first marketed in the US in 1973 and had been administered in combination with phentermine to prevent and treat obesity. However, in 1997, it was withdrawn from the US market as its use was associated with the onset of cardiac valvular fibrosis and pulmonary hypertension. Subsequently, the drug was withdrawn from sale globally and is no longer indicated for use in any therapeutic area anywhere in the world.

Despite the health concerns surrounding fenfluramine, attempts have been made to identify further therapeutic uses for that product. Aicardi and Gastaut (New England Journal of Medicine (1985), 313:1419 and Archives of Neurology (1988) 45:923-925) reported four cases of self-induced photosensitive seizures that responded to treatment with fenfluramine.

Clemens, in Epilepsy Research (1988) 2:340-343 reported a study on a boy suffering pattern sensitivity-induced seizures that were resistant to anticonvulsive treatment. Fenfluramine reportedly successfully terminated these self-induced seizures and the author concluded that this was because fenfluramine blocked the photosensitive triggering mechanism.

In Neuropaediatrics, (1996); 27(4):171-173, Boel and Casaer reported on a study on the effects of fenfluramine on children with refractory epilepsy. They concluded that when fenfluramine was administered at a dose of 0.5 to 1 mg/kg/day, this resulted in a reduction in the number of seizures experienced by the patients.

In a letter to Epilepsia, published in that journal (Epilepsia, 43(2):205-206, 2002), Boel and Casaer commented that fenfluramine appeared to be of therapeutic benefit in patients with intractable epilepsy.

Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders, de novo gene mutations or degenerative disease, head injury or trauma, metabolic disorders, cerebrovascular accident and alcohol withdrawal.

Although the present invention has applicability with respect to a range of different types of epilepsies and epilepsy subtypes, it is more particularly focused on Dravet syndrome, Doose syndrome, infantile spasms, and Lennox-Gastaut syndrome. There are a large number of subtypes of epilepsy that have been characterized. For example, the most recent classification system adopted by the International League Against Epilepsy's (“ILAE”) Commission on Classification and Terminology provides the following list of epilepsy syndromes (See Berg et. al., “Revised terminology and concepts for organization of seizures,” Epilepsia, 51(4):676-685 (2010)):

I. Electroclinical syndromes arranged by age at onset:

A. Neonatal period (1. Benign familial neonatal epilepsy (BFNE), 2. Early myoclonic encephalopathy (EME), 3. Ohtahara syndrome),

B. Infancy (1. Epilepsy of infancy with migrating focal seizures, 2. West syndrome, 3. Myoclonic epilepsy in infancy (MEI), 4. Benign infantile epilepsy, 5. Benign familial infantile epilepsy, 6. Dravet syndrome, 7. Myoclonic encephalopathy in nonprogressive disorders),

C. Childhood (1. Febrile seizures plus (FS+) (can start in infancy), 2. Panayiotopoulos syndrome, 3. Epilepsy with myoclonic atonic (previously astatic) seizures, 4. Benign epilepsy with centrotemporal spikes (BECTS), 5. Autosomal-dominant nocturnal frontal lobe epilepsy (ADNFLE), 6. Late onset childhood occipital epilepsy (Gastaut type), 7. Epilepsy with myoclonic absences, 8. Lennox-Gastaut syndrome, 9. Epileptic encephalopathy with continuous spike-and-wave during sleep (CSWS), 10. Landau-Kleffner syndrome (LKS), Childhood absence epilepsy (CAE));

D. Adolescence—Adult (1. Juvenile absence epilepsy (JAE), 2. Juvenile myoclonic epilepsy (JME), 3 Epilepsy with generalized tonic-clonic seizures alone, 4. Progressive myoclonus epilepsies (PME), 5. Autosomal dominant epilepsy with auditory features (ADEAF), 6. Other familial temporal lobe epilepsies,

E. Less specific age relationship (1. Familial focal epilepsy with variable foci (childhood to adult), 2. Reflex epilepsies);

II. Distinctive constellations: A. Mesial temporal lobe epilepsy with hippocampal sclerosis (MTLE with HS), B. Rasmussen syndrome, C. Gelastic seizures with hypothalamic hamartoma, D. Hemiconvulsion-hemiplegia-epilepsy, E. Other epilepsies, distinguished by 1. presumed cause (presence or absence of a known structural or metabolic condition, then 2. primary mode of seizure onset (generalized vs. focal);

III. Epilepsies attributed to and organized by structural-metabolic causes:

Malformations of cortical development (hemimegaloencephaly, heterotopias, etc.),

Neurocutaneous syndromes (tuberous sclerosis complex, Sturge-Weber, etc.),

C. Tumor,

D. Infection,

E. Trauma;

IV. Angioma: A. Perinatal insults, B. Stroke, C. Other causes;

V. Epilepsies of unknown cause;

VI Conditions with epileptic seizures that are traditionally not diagnosed as a form of epilepsy per se; A. Benign neonatal seizures (BNS); and B. Febrile seizures (FS).

See Berg et al., “Revised terminology and concepts for organization of seizures,” Epilepsia, 51(4):676-685 (2010)).

As can be seen from, for example, Part V of that list, there are still subtypes of epilepsy that have not yet been fully characterized and thus, the list is far from complete. For subtypes that are classified as encephalopathies these conditions comprise a group of disorders in which seizure activity leads to progressive cognitive dysfunction.

Those skilled in the art will recognize that these subtypes of epilepsy are triggered by different stimuli, are controlled by different biological pathways and have different causes, whether genetic or environmental. In other words, the skilled artisan will recognize that teachings relating to one epileptic subtype are not necessarily applicable to other subtypes. This can include recognition that different epilepsy subtypes respond differently to different anticonvulsant drugs, where, for instance, one medicine may improve one condition while the same medicine may worsen another epilepsy condition.

Dravet syndrome is a rare and catastrophic form of intractable epilepsy that begins in infancy. Initially, in the first year of life the patient experiences prolonged seizures. In their second year, additional types of seizure begin to occur and this typically coincides with a developmental decline, possibly due to repeated seizures causing brain damage such as cerebral hypoxia. This then leads to poor development of cognition, language and motor skills.

Children with Dravet syndrome are likely to experience multiple seizures per day. Epileptic seizures are far more likely to result in death in sufferers of Dravet syndrome; approximately 10 to 15% of patients diagnosed with Dravet syndrome die in childhood, in some cases between two and four years of age. The mean age at death of patients is reported to be 8.7±9.8 years (SD), with 73% of deaths occurring before the age of 10 years, and 93% before the age of 20. Additionally, patients are at risk of numerous associated conditions including orthopedic developmental issues, impaired growth and chronic infections.

Of particular concern, children with Dravet syndrome are particularly susceptible to episodes of Status Epilepticus, a convulsive seizure lasting longer than 5 minutes. This severe and intractable condition is categorized as a medical emergency requiring immediate medical intervention, typically involving hospitalization for intravenous anticonvulsant medication and/or medically-induced coma. Status epilepticus can be fatal. It can also be associated with severe cerebral hypoxia, possibly leading to damage to brain tissue. Frequent hospitalizations of children with Dravet syndrome are clearly distressing, not only to the patient but also to family and caregivers.

The cost of care for Dravet syndrome patients is also high as the affected children require constant supervision and many require institutionalization as they reach teenage years.

At present, although a number of anticonvulsant therapies can be employed to reduce the instance of seizures in patients with Dravet syndrome, the results obtained with such therapies are typically poor and those therapies only effect partial cessation of seizures at best. Seizures associated with Dravet syndrome are typically resistant to conventional treatments. Further, many anticonvulsants such as clobazam and clonazepam have undesirable side effects, which are particularly acute and prominent in pediatric patients.

In addition, it may be undesirable to treat the patient with any sodium channel drugs that are particularly undesirable when treating patients with Dravet syndrome.

It has been found that a certain class of drugs that are widely used in treating epilepsy, namely sodium channel blockers including carbamazepine, oxcarbazepine, lamotrigine, lacosamide, rufinamide, phenytoin, and fosphenytoin are contra-indicated in Dravet syndrome. These drugs have been found to lead to a greater incidence of seizures in almost all Dravet syndrome patients. Similarly, selective GABA reuptake inhibitors/GABA transaminase (“GABA T”) inhibitors including vigabatrin and tiagabine should be avoided in Dravet syndrome.

Sodium channel blockers preferentially affect the sodium channel at a specific stage of its cycle of rest, activation and inactivation, often by delaying the recovery from the inactivated state, thereby producing a cumulative reduction of Na+.

Non-epileptic brains have a natural balance of excitation (that can evoke seizures) and inhibition (that can reduce seizures). In epilepsies that are caused by too much excitatory neurotransmission (many of the epilepsies except SCN1A mutation related epilepsies), sodium channel blockers are beneficial because they reduce the neurotransmitters that cause too much excitation.

The most common mutation associated with Dravet syndrome is in the SCNA1 gene; the gene codes for the alpha-1 subunit of the sodium ion channel (Nav1.1), containing 2,009 amino acids, primarily expressed in inhibitory neurons. At least 70-80% of patients with Dravet syndrome have SCN1A mutations in the gene's exon which cause a loss of sodium channel function. Dravet has suggested as high as 85% have an SNC1A mutation (Dravet C. The core Dravet syndrome phenotype. Epilepsia 2011; 52 (Suppl. 2): 3-9). Some researchers predict that since only coding regions of the SCN1A gene are sequenced it is likely that many of the remaining patients harbor mutations in regulatory regions of the gene (outside of the coding sequences) that impair or prevent expression of this channel. Complete loss-of-function mutations in NaV1, encoded by SCNA1, cause Dravet Syndrome, which involves severe, intractable epilepsy and comorbidities of ataxia, sleep disturbance, and cognitive impairment. Mice with loss-of function mutations in NaV1.1 channels have severely impaired sodium currents and action potential firing in hippocampal GABAergic inhibitory neurons without detectable effect on the excitatory pyramidal neurons, which would cause hyperexcitability and contribute to seizures in Dravet Syndrome.

Impaired Nav1.1 channels, sodium currents and action potential firing are similarly impaired in the GABAergic Purkinje neurons in the cerebellum, which likely contributes to ataxia, and in the reticular nucleus of the thalamus and the suprachiasmatic nucleus of the hypothalamus, which likely contribute to circadian rhythm disturbances and sleep disorder. [Noebels et al., Jasper's Basic Mechanisms of the Epilepsies, 4th edition, Bethesda (Md.): National Center for Biotechnology Information (US); 2012].

Since mild loss-of-function mutations in NaV1.1 channels present a milder epilepsy phenotype called Familial Febrile Seizures, a unified loss-of-function hypothesis has been proposed for the spectrum of epilepsy syndromes caused by genetic changes in NaV1.1 channels: mild impairment predisposes to febrile seizures, intermediate impairment leads to GEFS+epilepsy, and severe loss of function causes the intractable seizures and co-morbidities of Dravet Syndrome. (Catterall WA, et al., NaV1.1 channels and epilepsy. J. Physiol. 2010; 588: 1849-59).

Experts in the field were surprised that haploinsufficiency (in which only one functional copy of the gene, as opposed to the usual two) is not enough to maintain healthy neuronal network function of a NaV channel causes epilepsy, because reduced sodium current should lead to hypoexcitability rather than hyperexcitability. The mechanistic basis for hyperexcitability and co-morbidities in Dravet Syndrome was studied using an animal model generated by targeted deletion or mutation of the SCN1A gene in mouse. Homozygous null NaV1.1(−/−) mice developed ataxia and died on postnatal day (P) 15 Ogiwara, et al., J. Neurosci. 2007;27:5903-5914, Yu, et al. Nat. Neurosci. 2006;9:1142-1149. Heterozygous NaV1.1(+/−) mice exhibited spontaneous seizures and sporadic deaths beginning after P21, with a striking dependence on genetic background.

The loss of NaV1.1 did not change voltage-dependent activation or inactivation of sodium channels in hippocampal neurons, however, the sodium current density was substantially reduced in inhibitory interneurons of NaV1.1(+/−) and NaV1.1(−/−) mice, but not in their excitatory pyramidal neurons. This reduction in sodium current caused a loss of sustained high-frequency firing of action potentials in hippocampal and cortical interneurons, thereby impairing their in vivo inhibitory function that depends on generation of high-frequency bursts of action potentials.

Based on the mechanism in which sodium channel blockers work to prevent seizure activity, one would think that these mutations in the SCN1A gene that cause the sodium channel to be ineffective (in essence, blocked) should prevent seizures and make a person with Dravet syndrome less prone to epilepsy. However, this loss of function is believed to lead to increased seizure activity, presumably because the result of this mutation is a decreased amount of inhibitory neurotransmitter that normally exists in the correct amount in the brain to balance excitatory neurotransmitters that make seizure more likely to occur. In this situation, the problem with the balance of excitation and inhibition in the brain is not too much excitation, it is too little inhibition. Giving sodium channel blocking drugs to Dravet syndrome patients further decreases the number of inhibitory neurotransmitters in the brain, tipping the balance toward more seizure activity.

Sodium channel blocker drugs which may be contradicted in connection with the present invention may include the following: phenytoin, carbamazepine, lamotrigine, oxcarbazepine, rufinamide, lacosamide, eslicarbazepine acetate, and phosphenytoin.

Stiripentol is approved in Europe, Canada, Japan and Australia and was approved recently by the US FDA, for the treatment of Dravet syndrome. Possible mechanisms of action of stiripentol include direct effects mediated through the gamma-aminobutyric acid (GABA)A receptor and indirect effects involving inhibition of cytochrome P450 activity with resulting increase in blood levels of clobazam and its active metabolite. Stiripentol is labeled for use in conjunction with clobazam, and other antiepileptic drugs may be added such as valproate. However, concerns remain regarding the use of stiripentol due to its inhibitory effect on hepatic cytochrome P450 enzymes. Further, the interactions of stiripentol with a large number of drugs means that combination therapy (which is typically required for patients with Dravet syndrome) is problematic. Additionally, the effectiveness of stiripentol is limited, with few if any patients ever becoming seizure free.

Polypharmacy, the use of two or more anti-epileptic drugs, for the treatment of Dravet syndrome can result in a significant patient burden, as the side effects, or adverse events from the multiple medications can be additive, and result in limiting the effectiveness of the therapy due to intolerability; in other words the small benefit of a medication may not outweigh the risk or negative effects the drug is having on the patient.

Available antiepileptic drugs do not offer adequate seizure control and respective neurosurgical procedures are not an option. New treatments for Dravet syndrome remain an important unmet need despite some level of efficacy in clinical trials for cannabidiol (Epidiolex®) and stiripentol (Diacomit®), which can be associated with cognitive or appetite safety concerns, respectively. Baraban, S, et al., Brain, 140 (3), p. 669-683 (March 2017).

Thus, there is accordingly a need to provide an improved method for treating or preventing Dravet syndrome and/or for treating, preventing and/or ameliorating seizures experienced by sufferers of Dravet syndrome.

### SUMMARY OF THE INVENTION

The invention is a method or a formulation for use in treating a patient diagnosed with Dravet syndrome which comprises administering to a patient a therapeutically effective dose of fenfluramine or a pharmaceutically acceptable salt, base, acid or amine thereof which includes fenfluramine hydrochloride in a liquid formulation at a concentration of 1.25 mg/ml, 2.5 mg/ml or 5 mg/ml and providing that to the patient over a period of days, weeks or months on a once a day, twice a day, three times a day or four times a day basis wherein the dose is provided to the patient at a level of 0.2 mg/kg/day or 0.8 mg/kg/day up to a maximum of 30 mg per day. The dosing is preferably provided at twelve-hour intervals twice a day whereby an aspect of the invention is to reduce convulsive seizure frequency by 50% or more, 60% or more, 70% or more, 80% or more, 90% or more, 95% or more, or completely eliminate seizures in the patient over a period of 10 days, 20 days, 30 days, 50 days, 100 days or more.

In an aspect of the invention, the fenfluramine is the sole therapeutic agent administered to the patient.

In another aspect of the invention, the fenfluramine is adjunctive therapy and is co-administered with a second, or a second and third, or a second, third and fourth, therapeutic agent. Any second, or any combination of second and third, or any combination of second, third and fourth therapeutic agents of interest may be utilized. In some cases, the second, or a second and third, or a second, third and fourth, therapeutic agent is selected from the group consisting of cannabidiol, carbamazepine, ethosuximide, fosphenytoin, lamotrigine, levetiracetam, phenobarbital, topiramate, stiripentol, valproic acid, valproate, verapamil, and benzodiazepines such as clobazam, clonazepam, diazepam, lorazepam, and midazolam and a pharmaceutically acceptable salt or base thereof.

In another aspect of the invention, the treatment continues in amounts and over a period of time so as to reduce the need by the patient for rescue medication by 25% or more, 50% or more, 75% or more, or completely eliminate the need for rescue medication.

In another aspect of the invention, the treatment is continued in amounts and over a period of time so as to reduce the patient's hospitalization visits by 25% or more, 50% or more, 75% or more, or completely eliminate hospitalization visits due to seizures.

Another aspect of the invention comprises administering a liquid fenfluramine formulation by the use of an oral syringe which is graduated for precise measurement of the liquid formulation. The formulation may include flavoring and coloring agents or may be completely devoid of any excipient materials beyond those necessary to dissolve the fenfluramine in the liquid which may be water.

In some cases, it can be desirable to test the patients for a genetic mutation prior to administration of some of the therapeutic agents provided by the disclosure, especially in cases where use of specific agent is contraindicated either because the agent is ineffective or because it would have undesired or serious side effects. Thus, it is in some cases desirable to test patients prior to treatment. In the case of patients having Dravet syndrome, testing can be carried out for mutations in the SCN1A (such as partial or total deletion mutations, truncating mutations and/or missense mutations e.g. in the voltage or pore regions S4 to S6), SCN1 B (such as the region encoding the sodium channel β1 subunit), SCN2A, SCN3A, SCN9A, GABRG2 (such as the region encoding the γ2 subunit), GABRD (such as the region encoding the σ subunit) and I or PCDH19 genes have been linked to Dravet syndrome.

In some instances, the mutations occur in genes that are linked diseases and conditions characterized by various seizure types including, for example, generalized seizures, myoclonic seizures, absence seizures, and febrile seizures. Mutations may occur in one or more of the following genes: ALDH7A1, CACNA1A, CACNA1H, CACNB4, CASR, CHD2, CHRNA2, CHRNA4, CHRNB2, CLCN2, CNTN2, CSTB, DEPDC5, EFHC1, EPM2A, GABRA1, GABRB3, GABRD, GABRG2, GOSR2, GPR98, GRIN1, GRIN2A, GRIN2B, KCNMA1, KCNQ2, KCNQ3, KCTD7, MBD5, ME2, NHLRC1, PCDH19, PRICKLE1, PRICKLE2, PRRT2, SCARB2, SCN1A, SCN1B, SCN2A, SCN4A, SCN9A, SLC2A1, TBC1D24.

In some instances, the mutations occur in genes that are linked to age-related epileptic encephalopathies including, for example, early infantile epileptic encephalopathy. Mutations may occur in one or more of the following genes: ALDH7A1, ARHGEF9, ARX, CDKL5, CNTNAP2, FH, FOXG1, GABRG2, GRIN2A, GRIN2B, KCNT1, MAGI2, MAPK10, MECP2, NRXN1, PCDH19, PLCB1, PNKP, PNPO, PRRT2, RNASEH2A, RNASEH2B, RNASEH2C, SAMHD1, SCN1A, SCN1B, SCN2A, SCN8A, SCN9A, SLC25A22, SLC2A1, SLC9A6, SPTAN1, STXBP1, TCF4, TREX1, UBE3A, ZEB2.

In some instances, the mutations occur in genes that are linked to malformation disorders including, for example, neuronal migration disorders, severe microcephaly, pontocerebellar hypoplasia, Joubert syndrome and related disorders, holoprosencephaly, and disorders of the RAS/MAPK pathway. Mutations may occur in one or more of the following genes: AHI1, ARFGEF2, ARL13B, ARX, ASPM, ATR, BRAF,C12orf57, CASK, CBL, CC2D2A, CDK5RAP2, CDON, CENPJ, CEP152, CEP290, COL18A1, COL4A1, CPT2, DCX, EMX2, EOMES, FGF8, FGFR3, FKRP, FKTN, FLNA, GLI2, GLI3, GPR56, HRAS, INPP5E, KAT6B, KRAS, LAMA2, LARGE, MAP2K1, MAP2K2, MCPH1, MED17, NF1, NPHP1, NRAS, OFD1, PAFAH1B1, PAX6, PCNT, PEX7, PNKP, POMGNT1, POMT1, POMT2, PQBP1, PTCH1, PTPN11, RAB3GAP1, RAF1, RARS2, RELN, RPGRIP1L, SHH, SHOC2, SIX3, SLC25A19, SNAP29, SOS1, SPRED1, SRD5A3, SRPX2, STIL, TGIF1, TMEM216, TMEM67, TSEN2, TSEN34, TSEN54, TUBA1A, TUBAE, TUBB2B, VDAC1, WDR62,VRK1, ZIC2.

In some instances, the mutations occur in genes that are linked to epilepsy in X-linked intellectual disability. Mutations may occur in one or more of the following genes: ARHGEF9, ARX, ATP6AP2, ATP7A, ATRX, CASK, CDKL5, CUL4B, DCX, FGD1, GPC3, GRIA3, HSD17B10, IQSEC2, KDM5C, MAGT1, MECP2, OFD1, OPHN1, PAK3, PCDH19, PHF6, PLP1, PQBP1, RAB39B, SLC16A2, SLC9A6, SMC1A, SMS, SRPX2, SYN1, SYP.

In some instances, the mutations occur in genes that are linked to storage diseases and conditions characterized by organelle dysfunction including, for example, neuronal ceroid lipofuscinosis, lysosomal storage disorders, congenital disorders of glycosylation, disorders of peroxisome biogenesis, and leukodystrophies. Mutations may occur in one or more of the following genes: AGA, ALG1, ALG12, ALG2, ALG3, ALG6, ALG8, ALG9, ALG11, ALG13, ARSA, ARSB, ASPA, B4GALT1, CLN3, CLN5, CLN6, CLN8, COG1, COG4, COG5, COG6, COG7, COGS, CTSA, CTSD, DDOST, DOLK, DPAGT1, DPM1, DPM3, EIF2B1, EIF2B2, EIF2B3, EIF2B4, EIF2B5, FUCA1, GALC, GALNS, GFAP, GLB1, GNE, GNPTAB, GNPTG, GNS, GUSB, HEXA, HEXB, HGSNAT, HYAL1, IDS, IDUA, MCOLN1, MFSD8, MGAT2, MLC1, MOGS, MPDU1, MPI, NAGLU, NEU1, NOTCH3, NPC1, NPC2, PEX1, PEX12, PEX14, PEX2, PEX26, PEX3, PEX5, PEX6, PEX7, PEX10, PEX13, PEX16, PEX19, PGM1, PLP1, PMM2, PPT1, PSAP, RFT1, RNASEH2A, RNASEH2B, RNASEH2C, SAMHD1, SDHA, SGSH, SLC17A5, SLC35A1, SLC35A2, SLC35C1, SMPD1, SUMF1, TMEM165, TPP1, TREX1.

In some instances, the mutations occur in genes that are linked to syndromic disorders with epilepsy including, for example, juvenile myoclonic epilepsy, childhood absence epilepsy, benign rolandic epilepsy, Lennox-Gastaut syndrome, Dravet syndrome, Ohtahara syndrome, West syndrome, etc. Mutations may occur in one or more of the following genes: ATP2A2, ATP6V0A2, BCKDK, CACNA1A, CACNB4, CCDC88C, DYRK1A, HERC2, KCNA1, KCNJ10, KIAA1279, KMT2D, LBR, LGI1, MAPK10, MECP2, MEF2C, NDE1, NIPBL, PANK2, PIGV, PLA2G6, RAIL RBFOX1, SCN8A, SERPINI1, SETBP1, SLC1A3, SLC4A10, SMC3, SYNGAP1, TBX1, TSC1, TSC2, TUSC3, UBE3A, VPS13A, VPS13B.

In some instances, the mutations occur in genes that are linked to the occurrence of migraines. Mutations may occur in one or more of the following genes: ATP1A2, CACNA1A, NOTCH3, POLG, SCN1A, SLC2A1.

In some instances, the mutations occur in genes that are linked to Hyperekplexia. Mutations may occur in the following genes: ARHGEF9, GLRA1, GLRB, GPHN, SLC6A5.

In some instances, the mutations occur in genes that are linked to inborn errors of metabolism including, for example, disorders of carbohydrate metabolism, amino acid metabolism disorders, urea cycle disorders, disorders of organic acid metabolism, disorders of fatty acid oxidation and mitochondrial metabolism, disorders of porphyrin metabolism, disorders of purine or pyridine metabolism, disorders of steroid metabolism, disorders of mitochondrial function, disorders of peroxisomal function, and lysosomal storage disorders. Mutations may occur in one or more of the following genes: ABAT, ABCC8, ACOX1, ACY1, ADCK3, ADSL, ALDH4A1, ALDH5A1, ALDH7A1, AMT, ARG1, ATIC, ATP5A1, ATP7A, ATPAF2, BCS1L, BTD, C120RF65, CABC1, COQ2, COQ9, COX10, COX15, DDC, DHCR7, DLD, DPYD, ETFA, ETFB, ETFDH, FOLR1, GAMT, GATM, GCDH, GCSH, GLDC, GLUD1, GLUL,HPD, HSD17B10, HSD17B4, KCNJ11, L2HGDH, LRPPRC, MGME1, MMACHC, MOCS1, MOCS2, MTHFR, MTR, MTRR, NDUFA1, NDUFA2, NDUFAF6, NDUFS1, NDUFS3, NDUFS4, NDUFS7, NDUFS8, NDUFV1, PC, PDHA1, PDHX, PDSS1, PDSS2, PGK1, PHGDH, POLG, PRODH, PSAT1, QDPR, RARS2, SCO2, SDHA, SLC19A3, SLC25A15, SLC46A1, SLC6A8, SUCLA2, SUOX, SURF1, TACO1,TMEM70, VDAC1.

Other genetic tests can be carried out and/or can be required as a condition of treatment.

In a preferred embodiment, the one or more targets are selected from the group consisting of the sigma-1 receptor, the 5-HT_{1A} receptor, the 5-HT_{1D} receptor, the 5-HT_{2A} receptor, the 5-HT_{2C} receptor, and the SERT transporter.

An aspect of the invention is a method of treating a patient diagnosed with epilepsy, comprising:

determining the patient had previously failed treatment with stiripentol;

administering to the patient a therapeutically effective compound of fenfluramine or a pharmaceutically acceptable salt, base or acid thereof;

repeating the administration over a period of 4 weeks or more until a reduction in seizure frequency is observed.

An aspect of the invention is a method of reducing seizures a patient, comprising:

determining the patient had previously failed treatment with stiripentol, based on lack of efficacy;

administering to the patient a therapeutically effective compound of fenfluramine or a pharmaceutically acceptable salt, base or acid thereof;

repeating the administration until the patient's seizures are eliminated over a period of 10 days or more until more until a reduction in seizure severity or frequency is observed.

An aspect of the invention is a method of reducing the severity or frequency of seizures in a patient, comprising:

determining the patient had previously failed treatment with stiripentol, based on tolerability;

administering to the patient a therapeutically effective compound of fenfluramine or a pharmaceutically acceptable salt, base or acid thereof;

repeating the administration over a period of 4 weeks or more by administering the fenfluramine twice per day in a liquid formulation until the patient's seizures are eliminated over a period of 10 days or more.

An aspect of the invention is a method of treating a patient diagnosed with Dravet syndrome, comprising:

determining the patient had previously failed treatment with stiripentol, based on lack of efficacy or tolerability;

administering to the patient a therapeutically effective compound of fenfluramine or a pharmaceutically acceptable salt, base or acid thereof;

repeating the administration over a period of 4 weeks or more by administering the fenfluramine twice per day in a liquid formulation in an amount of 0.2 mg/kg/day to 0.8 mg/kg/day until the patient's seizures are eliminated over a period of 10 days or more.

### BRIEF DESCRIPTION OF THE DRAWINGS

FIG. 1 is a flow diagram that schematically shows a study design for a prospective merged analysis of two identical double-blind, placebo-controlled studies. These two studies both involve administering a liquid solution of fenfluramine hydrochloride as an oral solution and are specifically referenced as ZX008-1501(US/Canada) and ZX008-1502(Europe/Australia) which are referred to collectively as Study 1 herein.

FIG. 2 is a chart showing baseline data and demographics for the fenfluramine study 1501 (see FIG. 1 above).

FIG. 3 is a chart showing the number of concomitant anti-epileptic drugs (AEDs) per subject in the placebo and treatment groups.

FIG. 4 is a chart showing the most common concomitant AEDs administered in the placebo and treatment groups.

FIG. 5 is a chart showing the most common prior AEDs administered in the placebo and treatment groups.

FIG. 6 is two bar graphs showing the percent difference from placebo in reduction in mean monthly (28 days) convulsive seizures for the two-week titration period plus the twelve-week maintenance period. Study 1 met primary endpoint demonstrating ZX008, at a dose of 0.8 mg/kg/day, is superior to placebo as adjunctive therapy in the treatment of Dravet syndrome based on the change in the mean monthly convulsive seizure frequency (p<0.001). ZX008, at a dose of 0.2 mg/kg/day, also demonstrated superiority to placebo based on the same endpoint (p=0.019). P-values are treatment compared with placebo group.

FIG. 7 is a chart showing the mean and median percent reduction in seizures at various time points for placebo and treatment groups. The mean is affected by outliers more than the median. In a model used to compare treatment to placebo, baseline and post-treatment seizure frequency were log-transformed to diminish the effect of outliers and produce more symmetrically distributed data. The model yields an adjusted geometric mean, which lies between the arithmetic mean and the median. The percentage difference in the geometric mean (aka Least Squares Mean from the model) between 0.8 and placebo is 63.9%-between the arithmetic and geometric mean differences. The model used treatment group and age group ≥2 years to <6 years, and 6 years and older) as factors and log baseline as covariate.

FIG. 8 is a chart showing convulsive seizure frequency values at various time points for placebo and treatment groups.

FIG. 9 is three bar graphs showing the mean of convulsive seizure frequencies for placebo and treatment groups.

FIG. 10 is six bar graphs showing convulsive seizure responder rates during the titration period and the maintenance period for placebo and treatment groups. The proportion of patients who achieved ≥50% and ≥75% reduction in mean monthly convulsive seizures during the two-week titration period and the twelve-week maintenance period is shown. P-values calculated vs. placebo.

FIG. 11 is a graph that illustrates the percent reduction in seizure frequency during the titration period and the maintenance period for placebo and treatment groups.

FIG. 12 is a six bar graph which shows the median and mean of each subject's longest seizure free interval for the combined two week titration period and twelve week maintenance period. P-values are for median values for both treatment groups vs. placebo.

FIG. 13 are bar graphs which show the proportion of subjects who experienced seizure freedom or near seizure freedom in the placebo and treatment groups. The proportion of subjects who experienced zero (0) seizures or one (1) seizure throughout the full treatment period (two-week titration period and twelve-week maintenance period) is shown. Mean monthly seizure rate at baseline for all patients in Study 1 was 40/month.

FIG. 14 is a graph showing the longest seizure free interval by quartile. The calculation designated minimum duration as one day.

FIG. 15 provides a table of Clinical Global Impression-I (CGI-I) scale values given by an investigator for placebo and treatment groups.

FIG. 16 are bar graphs showing a visual representation of CGI-I ratings given by an investigator for placebo and treatment groups.

FIG. 17 provides a table of CGI-I values given by a parent or caregiver for placebo and treatment groups.

FIG. 18 are bar graphs showing a visual representation of CGI-I ratings given by a parent or caregiver for placebo and treatment groups.

FIG. 19 provides a table of treatment emergent adverse events for placebo and treatment groups.

FIG. 20 provides a table of treatment emergent adverse events for placebo and treatment groups.

FIG. 21 consists of FIG. 21A, FIG. 21B and FIG. 21C which provides a table of treatment emergent adverse events for placebo and treatment groups.

FIG. 22 provides a table of the numbers of subjects with treatment emergent adverse events and treatment emergent serious adverse events. Prospective cardiac safety monitoring throughout the study demonstrated no clinical or echocardiographic evidence of cardiac valvulopathy or pulmonary hypertension. The formulation was generally well-tolerated with adverse events consistent with the known safety profile of fenfluramine. The incidence of treatment emergent adverse events was higher in treatment groups as compared to placebo; however, the incidence of treatment emergent serious adverse events was similar in all three groups. Five subjects in the 0.8 mg/kg/day group had an adverse event leading to study discontinuation, compared to zero in the other treatment groups.

FIG. 23 summarizes information relating to Study 1, specifically noting the number of subjects that withdrew during the trial.

FIG. 24 summarizes adverse events and lists the most common adverse events.

FIG. 25 summarizes the cardiac ECHO (echocardiogram) findings of the Study 1 study.

FIG. 26 summarizes conclusions of Study 1.

FIG. 27 consists of Table 27a, 27b, 27c, 27d, 27e and 27f which show specific numbered data for the trial described here with respect to the placebo group and the 0.2 mg treatment group and 0.8 mg treatment group.

FIG. 28 is a graph showing the percent reduction in convulsive seizure frequency over a 28-day period for the Study 1 study the upper line showing the 0.8 mg/kg/day group and the middle lane showing the 0.2 mg/kg/day group and the lower line showing the placebo group.

Note that the FIGS. 1-28 and 36 are directed to Study 1 that is further described in Examples 2, 3 and 4 in the Detailed Description.

FIG. 29 is a schematic representation summarizing the study design for Study 1504 as described in Example 5.

FIG. 30 summarizes information on the proportion of patients who achieved ≥50% and ≥75% reductions in mean monthly convulsive seizures.

FIG. 31 summarizes information on the longest seizure free interval in both treatment and placebo arms of the study and presents the data as both the median and mean calculated values.

FIG. 32 summarizes information on the percentage of patients who experienced zero or one seizure throughout the full treatment period.

FIG. 33 summarizes information on seizure frequency throughout the treatment period and demonstrates that the treatment effect was durable across the full fifteen-week period and demonstrates no tachyphylaxis, a diminishing response to successive doses of a drug and is common in drugs acting on the nervous system.

FIG. 34 summarizes some of the findings relating to safety in Study 1504.

FIG. 35 summarizes adverse reactions occurring in ≥5 patients treated with ZX008 in Study 1 (0.2 mg/kg/day and 0.8 mg/kg/day; maximum 30 mg/day) and in Study 1504 (0.5 mg/kg/day in conjunction with stiripentol wherein the dose was titrated at 0.2 mg/kg/day increments maximum 20 mg/day) and pooled placebo.

FIG. 36 is a graph showing the reductions in non-convulsive seizures

### DETAILED DESCRIPTION OF THE INVENTION

Before the present methods of treatment are described, it is to be understood that this invention is not limited to particular method described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a step of administering” includes a plurality of such steps and reference to “the symptom” includes reference to one or more symptoms and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention.

Further, the dates of publication provided may be different from the actual publication dates which may need to be independently confirmed.

### Definitions

The term “reduction from baseline” is used throughout in order to refer to a reduction relative to the same or similar patient prior to administration of fenfluramine. During the baseline period, the patient is treated with other therapeutic agents, except for fenfluramine. Treatment with the same other therapeutic agents is substantially maintained during the treatment with fenfluramine. The comparison is made relative to the observations, measurements or tests made during the baseline period.

The term “fenfluramine” refers to both the free-base depicted in Structure 1 and its pharmaceutically acceptable acid addition salts. Pharmaceutically acceptable acid addition salts are those formed from acids which form non-toxic acid anions such as, for example, the hydrochloride, hydrobromide, sulphate, phosphate or acid phosphate, acetate, maleate, fumarate, lactate, tartrate, citrate and gluconate salts.

The term “ZX008” refers to fenfluramine hydrochloride formulated as an oral solution.

### Specific Aspects of the Invention

After many years of extensive research, it has unexpectedly been found that fenfluramine can administered as described here to reduce or eliminate seizures in patients with Dravet syndrome. This is confirmed by the results presented herein. Additional information is in the article by Ceulemans et al., Epilepsia (2012) 53(7):1131-1139, the contents of which are incorporated herein.

For the avoidance of doubt, the term “prevention” of seizures means the total or partial prevention (inhibition) of seizures. Ideally, the methods of the present invention result in a total prevention of seizures; indeed, this ideal has been achieved in a number of patients treated by the inventors. However, the invention also encompasses methods in which the instances of seizures are decreased by at least 50%, at least 60%, at least 70%, at least 80% or at least 90%.

It is known that patients with Dravet syndrome commonly experience photosensitive or induced seizures. From teachings in the prior art, e.g. Aicardi and Gastaut (1988) and Boel and Casaer (1996)—both discussed above—it might have been expected that fenfluramine would reduce photosensitive or induced seizures. Importantly, however, it has surprisingly been found that all types of seizures exhibited by patients with Dravet syndrome, that is seizures in addition to and other than those that are photosensitive or induced, convulsive and non-convulsive can be suppressed by treatment in accordance with a method of the present invention. Convulsive seizures involve the entire body and are involuntary; they include a sudden onset of very evident, intense rapid muscle contraction (tonic phase) and followed by jerking of extremities (clonic phase) of body muscles, and also may include shaking, loss of consciousness, difficulty breathing, loss of bowel/bladder control and/or confusion. usually lasting a few minutes. Atonic seizures are a type of seizure that causes sudden loss of muscle strength, also called akinetic seizures, drop attacks or drop seizures in which the sudden lack of muscle strength, or tone, can cause the person to fall to the ground and are typically classified as a type of motor seizure. Atonic seizures occur commonly in patients having Lennox Gastaut syndrome. The affected person usually remains conscious and may not fall, but may exhibit head drop, drooping eyelids, or they may drop anything they were holding.

Seizures that lack clonic or tonic activity or other major motor activity are classified as non-convulsive and they may range from being readily apparent to being nearly undetectable by an observer. Non-motor focal seizures with or without impaired awareness can involve sensory, cognitive, emotional or autonomic abnormalities depending on the area of the brain experiencing seizure activity.

Atypical absence seizures are so named because they are of longer duration and have a slower onset and offset than absence seizures (i.e., the more usual sort of impaired awareness seizure) and involve different symptoms. Atypical absence seizures may begin with staring into space, usually with a blank look accompanied usually by a change in muscle tone and movement. Repetitive blinking may occur which appears as rapid fluttering of the eyelids. Automatisms such as smacking of the lips or chewing movements, rubbing fingers together or making other hand motions may also occur which are not under the voluntary control of the patient. An atypical absence seizure can last up to 20 seconds or more.

Thus, in context of the present invention, the term “seizure” is used to not only encompass photosensitive or induced seizures, but some or all of the other types of seizures experienced by patients with epilepsy

Moreover, fenfluramine's therapeutic effects appear to be independent of any significant placebo effects. In general, the effects of the placebo arm in epilepsy clinical trials are generally quite positive, making an efficacious therapy difficult to validate. While seizure-freedom rates on placebo are quite low (0-2.8%), rates on 50%-responder rates on placebo are quite a bit larger (4-27%) (Burneo et al., 2002; Cramer et al., 1999; Guekht et al., 2010; Rheims et al., 2008; Zaccara et al., 2015), and may be higher yet due to a statistically significant publication bias in epilepsy public trials (Beyenburg et al., 2010). Although the placebo phenomenon may be partially attributable to normal disease progression (Goldenholz et al., Ann. Neurol. 2015 SEP; 78(3): 329-336. Published online 2015 Jul. 29, doi 10.1002/ana.24470), and its magnitude influenced by a number of factors, it is verifiable, and likely due to positive or negative expectations of patients and of investigators.

See generally Goldenholz et al., Response to Placebo in Clinical Epilepsy Trials—Old Ideas and New Insights Epilepsy Res. 2016 May; 122: 15-25, Published online 2016 Feb. 10. doi: 10.1016/j.eplepsyres.2016.02.002.

Unexpectedly, the results obtained in double-blinded fenfluramine clinical trials effectively match those from open label studies, which leads to the surprising conclusion that fenfluramine's efficacy is free of any placebo effect, unlike the majority of more conventional anti-epileptics. This is an unexpected and surprising result providing improvements in the reliability and robustness of fenfluramine's efficacy as an antiseizure medication in Dravet syndrome.

Thus, according to a further aspect of the present invention, there is provided a method of preventing or reducing seizures in a patient diagnosed with Dravet syndrome by administering to that patient a therapeutically effective dose of fenfluramine, whereby seizures are prevented or reduced. In various embodiments of this aspect, the instances of seizures are decreased by at least 50%, at least 60%, at least 70%, at least 80% or at least 90%.

Thus, according to a further aspect of the present invention, there is provided a method of treating a patient that exhibits a mutation in one, some or all of the above genes by administering to that patient an effective dose of fenfluramine. In certain embodiments of this aspect of the invention, the patient has been diagnosed with Dravet syndrome.

Fenfluramine has been known to inhibit serotonin reuptake and to trigger the release of serotonin in the brain due to disruption of its vesicular storage. Data from more recent studies provide evidence that fenfluramine is a positive allosteric modulator of the sigma-1 receptor. The results provided here indicate a high degree of efficacy in the treatment of Dravet syndrome using fenfluramine to dramatically reduce and in some cases completely eliminate seizures from patients being treated.

Thus, according to a still further aspect of the present invention, there is provided a method of stimulating or modulating one or more targets in the brain of a patient by administering a therapeutically effective dose of fenfluramine to said patient, wherein said one or more targets are selected from the group consisting of a chaperone protein, a bioamine transporter (BAT), and a 5HT receptor, wherein

(a) the chaperone protein is selected from the group consisting of the sigma-1 protein and the sigma-2 protein; and

(b) the BAT is selected from the serotonin transporter (SERT), the norepinephrine transporter (NET), and the dopamine transporter (SERT); and

(c) the 5-HT receptor is in a family of receptors selected from the group consisting of 5-HT1, 5-HT2, 5-HT3, 5-HT4, 5-HT5, 5-HT6, and 5-HT7; wherein
(i) the 5-HT receptor in the 5-HT1 receptor family is selected from the group consisting of 5-HT_{1A}, 5-HT_{1B}, and 5-HT_{1C}, 5-HT_{1D}, 5-HT_{1E}, and 5-HT_{1F};
(ii) the 5-HT receptor in the 5-HT2 receptor family is selected from the group consisting of 5-HT_{2A}, 5-HT_{2B}, and 5-HT_{2C};
(iii) the 5-HT receptor in the 5-HT3 receptor family is selected from the group consisting of 5-HT_{3A} and 5-HT_{3B};
(iv) the 5-HT receptor is 5-HT4;
(v) the 5-HT receptor in the 5-HT5 receptor family is selected from the group consisting of 5-HT_{5A} or 5-HT_{5B}; and
(vi) the 5-HT receptor in the 5-HT7 family is 5-HT7,

whereby the activity of the one or more targets in the brain of the patient are stimulated or modified.

In an embodiment, the stimulation of the one or more targets in a Dravet syndrome patient provides improvement in one or more symptoms of the disease chosen from reductions in (i) convulsive seizure frequency, ataxia, gait abnormality, sleep disturbances and cognitive impairment. Changes in ataxia can be measured for example by a clinical scale (SARA) developed by Schmitz-Hübsch et al. (Movement Disorders 2007, 22:1633-7) which assesses a range of different impairments in cerebellar ataxia. The scale is made up of 8 items related to gait, stance, sitting, speech, finger-chase test, nose-finger test, fast alternating movements and heel-shin test. Cognitive assessments in Dravet syndrome patients may be made using, for example, the BRIEF scale for measuring executive function or other measures such as those described by Ahca et al., in Child Neuropsychology, 21(5):693-715 (2014).

In a preferred embodiment, the one or more targets are selected from the group consisting of the sigma-1 receptor, the 5-HT_{1A} receptor, the 5-HT_{1D} receptor, the 5-HT_{2A} receptor, the 5-HT_{2C} receptor, and the SERT transporter.

In embodiments of the invention, any effective dose of fenfluramine can be employed. However, surprisingly low doses of fenfluramine have been found by the inventors to be efficacious, particularly for inhibiting or eliminating seizures in Dravet syndrome patients. Thus, in preferred embodiments of the invention, the maximum daily dose is not more than about 30 mg/day, with a daily dose of less than about 1.0 mg/kg/day, 0.9 mg/kg/day, 0.8 mg/kg/day, 0.7 mg/kg/day, 0.6 mg/kg/day, 0.5 mg/kg/day, about 0.4 mg/kg/day, about 0.3 mg/kg/day, about 0.25 mg/kg/day or about 0.2 mg/kg/day to about 0.1 mg/kg/day, about 0.05 mg/kg/day, or about 0.01 mg/kg/day is employed. Put differently, a preferred dose is not more than about 30 mg/day, and less than about 1 to about 0.01 mg/kg/day. Such a dose is less than the daily dose of fenfluramine suggested for administration to achieve weight loss.

The dose of fenfluramine administered in the methods of the present invention can be formulated in any pharmaceutically acceptable dosage form including, but not limited to oral dosage forms such as tablets including orally disintegrating tablets, capsules, lozenges, oral solutions or syrups, oral emulsions, oral gels, oral films, buccal liquids, powder e.g. for suspension, and the like; injectable dosage forms; transdermal dosage forms such as transdermal patches, ointments, creams; inhaled dosage forms; and/or nasally, rectally, vaginally administered dosage forms. Such dosage forms can be formulated for once a day administration, or for multiple daily administrations (e.g. 2, 3 or 4 times a day administration).

The dosage form of fenfluramine employed in the methods of the present invention can be prepared by combining fenfluramine with one or more pharmaceutically acceptable diluents, carriers, adjuvants, and the like in a manner known to those skilled in the art of pharmaceutical formulation.

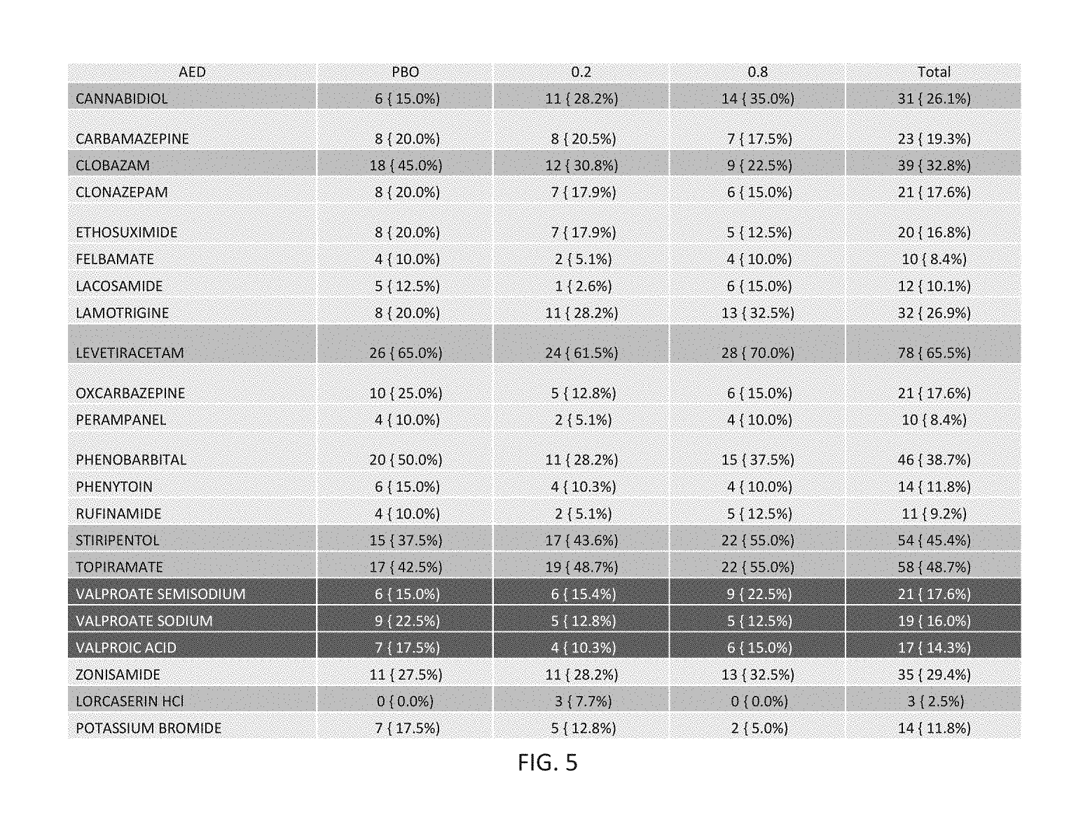

In a method of the present invention, fenfluramine can be employed as a monotherapy in the treatment of Dravet syndrome. Alternatively, fenfluramine can be co-administered simultaneously, sequentially or separately with one or more co-therapeutic agents, such as anticonvulsants. Possible co-therapeutic agents are listed in FIG. 5. Further, such agents can be selected from the group consisting of cannabidiol, carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, midazolam. Use of a pharmaceutically acceptable salt of a co-therapeutic agent is also contemplated. However, carbamazepine, oxcarbazepine, lamotrigine, phenytoin and vigabatrin are typically contraindicated in Dravet syndrome, as they tend to make seizures worse, rather than better.

Fenfluramine can be employed to treat a patient who has previously been treated with an anticonvulsant, e.g., as described herein, such as stiripentol or cannabidiol. In some instances, the patient is diagnosed with Dravet syndrome that is refractory to treatment with a particular anticonvulsant agent e.g., as described herein. In certain instances, the anticonvulsant agent is a modulator of neuronal GABA(A) receptors, such as stiripentol. By refractory to anticonvulsant agent (e.g., stiripentol or cannabidiol) is meant that the frequency of convulsive seizures (CSF) is not significantly reduced in the patient in response to therapy (e.g., monotherapy) with the anticonvulsant agent. In some cases, a significant reduction in CSF is a 10% or greater reduction in mean monthly convulsive seizures, such as 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, or 99% or greater reduction. In certain instances, the subject method is a method of preventing or treating seizures in a patient diagnosed with Dravet syndrome refractory to stiripentol by administering to that patient a therapeutically effective dose of fenfluramine, whereby seizures are prevented or reduced. In various embodiments of this aspect, the instances of seizures (e.g., mean monthly convulsive seizures) are decreased by at least 50%, at least 55%, at least 60%, at least 65%, at least 70%, at least 75%, at least 80%, at least 85%, at least 90%, or at least 95%.

The invention includes a use of a formulation for treating a patient diagnosed with Dravet syndrome, wherein the formulation comprises:

a therapeutically effective dose of fenfluramine or a pharmaceutically acceptable salt, base or acid thereof; and

wherein the use is for repeated administrations over a period of days until the patient exhibits a reduction from baseline in convulsive seizure frequency of 40% or more.

The invention includes a use of a formulation for treating a patient diagnosed with Dravet syndrome, wherein the formulation comprises:

a therapeutically effective dose of fenfluramine or a pharmaceutically acceptable salt, base or acid thereof in an amount of 0.2 mg/Kg/day or more, up to 30 mg/day;

a co-therapeutic agent; and

wherein the co-therapeutic agent and fenfluramine are in a liquid formulation for use in repeated daily administrations over a period of weeks until the patient exhibits a reduction from baseline in convulsive seizure frequency of 60% or more.

The invention includes a use as described throughout, wherein the fenfluramine is the only active ingredient administered to the patient.

The invention includes a use as described throughout, further comprising:

administering a co-therapeutic agent.

The invention includes a use as described throughout, wherein the co-therapeutic agent is selected from the group consisting of, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof.

In some aspects, provided herein is a method of reducing convulsive seizure frequency in a human patient diagnosed with Dravet syndrome or other epileptic encephalopathy, comprising administering to the patient a therapeutically effective dose of fenfluramine or a pharmaceutically acceptable salt, base, acid or amine thereof, and repeating the administering over a period of a day or days, weeks, months or years until the patient exhibits a significant reduction (e.g., 40%, 50% 60%, 70%, 80%, 90%, 95% or even greater) from baseline in convulsive seizure frequency. In some embodiments, the method further comprises repeating the administering until the patient is seizure free for a period of ≥1 day, or for a period of ≥9 days, or for a period of ≥14 days, or for a period of ≥21 days, or for a period of ≥14 weeks, or for a period of ≥6 months, or for a period of ≥1 year. In some embodiments, the method further comprises repeating the administering until the patient is permanently seizure free. In some embodiments of the method, convulsive seizures are completely eliminated for 10 days or more, 20 days or more, 30 days or more, 50 days or more, 100 days or more. In some embodiments of the method, the repeating administration continues over a period of 4 weeks or more until a significant reduction from baseline in convulsive seizure frequency is observed. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof. In some embodiments, the administering is over a period of months, and the co-therapeutic agent is clobazam. In some embodiments, the co-therapeutic agent is a combination of stiripentol, valproate and clobazam. Administration may be daily, once a day, twice a day, three times a day or four times a day. In some embodiments, the dose is provided to the patient at a level of 0.2 mg/kg/day or 0.8 mg/kg/day up to a maximum of 30 mg per day. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments, the treatment improves two or more symptoms selected from the group consisting of convulsive seizures, ataxias, gait abnormalities, sleep disturbances and cognitive impairment. In some aspects, the present disclosure provides a method of treating a patient diagnosed with Dravet syndrome, comprising administering to the patient a therapeutically effective dose of fenfluramine or a pharmaceutically acceptable salt, base or acid thereof in an amount of 0.2 mg/kg/day or more, up to 30 mg/day; administering a co-therapeutic agent; and repeating the administering of the co-therapeutic agent and fenfluramine over a period of weeks until the patient exhibits a reduction from baseline in convulsive seizure frequency of 60% or more. In some aspects, the present disclosure provides a use of a formulation for treating a patient diagnosed with Dravet syndrome, wherein the formulation comprises a therapeutically effective dose of fenfluramine or a pharmaceutically acceptable salt, base or acid thereof in an amount of 0.2 mg/Kg/day or more, up to 30 mg/day; a co-therapeutic agent; and wherein the co-therapeutic agent and fenfluramine are in a liquid formulation; for use over a period of weeks until the patient exhibits a reduction from baseline in convulsive seizure frequency of 60% or more. Pharmaceutical compositions and formulations for use in practicing the subject methods are also provided.

The invention includes a use of a formulation for treating a patient diagnosed with Dravet syndrome, wherein the formulation comprises:

a therapeutically effective dose of fenfluramine or a pharmaceutically acceptable salt, base or acid thereof;

wherein the use is for repeated administrations over a period of days until the patient exhibits an increase from baseline in an average time between convulsive seizures of eight hours or more.

The invention includes a use of a formulation for treating a patient diagnosed with Dravet syndrome, wherein the formulation comprises:

a therapeutically effective dose of fenfluramine or a pharmaceutically acceptable salt, base or acid thereof in an amount of 0.2 mg/Kg/day or more, up to 30 mg/day;

administering a co-therapeutic agent; and

wherein the co-therapeutic agent and fenfluramine are in a liquid formulation for use in repeated daily administrations over a period of weeks until the patient exhibits an increase from baseline in average time between convulsive seizures of one week or more.

The invention includes a use as described throughout, wherein the fenfluramine is the only active ingredient administered to the patient.

The invention includes a use as described throughout, further comprising:

administering a co-therapeutic agent.

The invention includes a use as described throughout, wherein the co-therapeutic agent is selected from the group consisting of, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof.

In some aspects, provided herein is a method of increasing an average time between seizures in a human patient diagnosed with Dravet syndrome or other epileptic encephalopathy, comprising administering to the patient a therapeutically effective dose of fenfluramine or a pharmaceutically acceptable salt, base, acid or amine thereof, and repeating the administering over a period of days until the patient exhibits an increase from baseline in average time between convulsive seizures of six hours or more, or an average time of eight hours or more, or an average time of one day or more, or an average time of two days or more, or an average time of one week or more, or an average time of one month or more. In some embodiments, a patient diagnosed with Dravet syndrome or other epileptic encephalopathy is treated by administering to the patient a therapeutically effective dose of fenfluramine or a pharmaceutically acceptable salt, base, acid or amine thereof, and repeating the administering over a period of a day or days, or over a period of weeks, months or years until the patient exhibits an increase from baseline in average time between convulsive seizures of 6 to 23 hours or more, 1 to 6 days or more, 1 to 3 weeks or more, 1 to 11 months or more, one year or more, or seizures are completely eliminated for 10 days or more, 20 days or more, 30 days or more, 50 days or more, 100 days or more. In some embodiments, the administering is repeated over a period of days until the patient exhibits an increase from baseline in average time between convulsive seizures of 4 hours or more, 5 hours or more, 6 hours or more, 7 hours or more, 8 hours or more, 9 hours or more, 12 hours or more, 15 hours or more, 18 hours or more, or 24 hours or more. In some embodiments, repeating the administering occurs over a period of a day or days, or over a period of weeks, or over a period of months or over a period of years. In some embodiments in which the repeat administration is daily, the administration is once a day, twice a day, three times a day or four times a day. In some embodiments, the dose is provided to the patient at a level of 0.2 mg/kg/day or 0.8 mg/kg/day up to a maximum of 30 mg per day. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof. In some embodiments, the administering is over a period of months, and the co-therapeutic agent is clobazam. In some embodiments, the co-therapeutic agent is a combination of stiripentol, valproate and clobazam. In some embodiments, the method further comprises repeating the administering over a period of weeks until the patient exhibits an increase from baseline in an average time between convulsive seizures of one month or more. In some embodiments, the method further comprises repeating the administering until the patient is seizure free for a period of ≥1 day, or for a period of ≥9 days, or for a period of ≥14 days, or for a period of ≥21 days, or for a period of ≥14 weeks, or for a period of ≥6 months, or for a period of ≥1 year. In some embodiments, the method further comprises repeating the administering until the patient is permanently seizure free. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml. In some embodiments, the fenfluramine hydrochloride in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments of the method, the repeating administration continues over a period of 4 weeks or more until an increase from baseline in average time between convulsive seizures of 6 to 23 hours or more, 1 to 6 days or more, 1 to 3 weeks or more, 1 to 11 months or more, one year or more, or seizures are completely eliminated for 10 days or more, 20 days or more, 30 days or more, 50 days or more, 100 days or more is observed. In some embodiments, the therapeutically effective dose of fenfluramine or a pharmaceutically acceptable salt, base, acid or amine thereof is twice per day in a liquid formulation in an amount of 0.2 mg/kg/day to 0.8 mg/kg/day. In some aspects, the present disclosure provides a method of treating a patient diagnosed with Dravet syndrome, by administering to the patient a therapeutically effective dose of fenfluramine or a pharmaceutically acceptable salt, base or acid thereof in an amount of 0.2 mg/kg/day or more, up to 30 mg/day; administering a co-therapeutic agent; and repeating the administering of the co-therapeutic agent and fenfluramine over a period of weeks until the patient exhibits an increase from baseline in average time between convulsive seizures of one week or more. In some aspects, the present disclosure provides a use of a formulation for treating a patient diagnosed with Dravet syndrome, wherein the formulation comprises a therapeutically effective dose of fenfluramine or a pharmaceutically acceptable salt, base or acid thereof in an amount of 0.2 mg/kg/day, up to 30 mg/day; a co-therapeutic agent; and wherein the co-therapeutic agent and fenfluramine are in a liquid formulation, for use over a period of weeks until the patient exhibits an increase from baseline in average time between convulsive seizures of one week or more. Pharmaceutical compositions and formulations for use in practicing the subject methods are also provided.

The invention includes a use of a formulation for treating a patient diagnosed with Dravet syndrome, wherein the formulation comprises:

a therapeutically effective dose of fenfluramine or a pharmaceutically acceptable salt, base or acid thereof;

wherein the use is for repeated administrations over a period of days until the patient exhibits a reduction from baseline in a seizure type experienced by the patient.

The invention includes a use of a formulation for treating a patient diagnosed with Dravet syndrome, wherein the formulation comprises:

a therapeutically effective dose of fenfluramine or a pharmaceutically acceptable salt, base or acid thereof in an amount of 0.2 mg/kg/day or more, up to 30 mg/day;

a co-therapeutic agent; and

wherein the co-therapeutic agent and fenfluramine are in a liquid formulation for use in repeated daily administrations over a period of weeks until the patient exhibits a reduction from baseline of two types of seizures.

The invention includes a use as described throughout, wherein the fenfluramine is the only active ingredient administered to the patient.

The invention includes a use as described throughout, further comprising:

administering a co-therapeutic agent.

The invention includes a use as described throughout, wherein the co-therapeutic agent is selected from the group consisting of, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof.

In some aspects, provided herein is a method of reducing a particular type of seizure in a human patient diagnosed with Dravet syndrome or other epileptic encephalopathy, by administering to the patient a therapeutically effective dose of fenfluramine or a pharmaceutically acceptable salt, base, acid or amine thereof, and repeating the administering over a period of a day or days, or over a period of weeks, months or years until the patient exhibits a significant reduction (e.g., 40%, 50% 60%, 70%, 80%, 90%, 95% or even greater) from baseline in seizures of a particular type. In some aspects, provided herein is a method of treating a patient diagnosed with Dravet syndrome, by administering to the patient a therapeutically effective dose of fenfluramine or a pharmaceutically acceptable salt, base or acid thereof; and repeating the administering over a period of days until the patient exhibits a reduction from baseline in a seizure type experienced by the patient. The reduction may be of one, two, three or multiple specific types of seizures. In some embodiments, two seizure types are reduced. In some embodiments, three seizure types are reduced. In some embodiments of the method, the seizure type reduced is selected from the group consisting of non-convulsive seizures, generalized seizures, myoclonic seizures, absence/atypical absence seizures, and febrile seizures, or any combination thereof. In some embodiments, particularly in Dravet syndrome, multiple seizure types are typically present including convulsive seizures consisting of generalized clonic seizures (GCS), generalized tonic-clonic seizures (prior terminology was grand mal), or alternating unilateral clonic seizures; myoclonic seizures; atypical absences and obtundation (dulled or impaired awareness) status; focal seizures, with or without secondary generalization; or, more rarely, tonic seizures. In some embodiments, the seizure type reduced is selected from the group consisting of photosensitive seizures and self-induced seizures. In some embodiments, the seizure type reduced is selected from atonic, or focal seizures without clear observable motor signs. In some embodiments, the method further comprises recording the seizure types experienced daily by the patient or caregiver in an electronic diary. In some embodiments, repeating the administering occurs over a period of a day or days, or over a period of weeks, or over a period of months or over a period of years. In some embodiments in which the repeat administration is daily, the administration is once a day, twice a day, three times a day or four times a day. In some embodiments, the dose is provided to the patient at a level of 0.2 mg/kg/day or 0.8 mg/kg/day up to a maximum of 30 mg per day. In some embodiments, the method further comprises repeating the administering of the fenfluramine in an amount of 0.2 mg/kg/day or more up to 30 mg/day until the patient no longer experiences at least one type of seizure experienced by the patient prior to administering the fenfluramine. In some embodiments, the method further comprises repeating the administering of the fenfluramine in an amount of 0.2 mg/kg/day or more up to 30 mg/day until the patient improves two or more symptoms selected from the group consisting of convulsive seizures, ataxias, gait abnormalities, sleep disturbances and cognitive impairment. In some embodiments, the patient exhibits a reduction from baseline in a particular seizure type of 50% or more, 60% or more, 70% or more, 80% or more, 90% or more, 95% or more. In some embodiments, the particular seizure type is completely eliminated for 10 days or more, 20 days or more, 30 days or more, 50 days or more, 100 days or more. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof. In some embodiments, the administering is over a period of months, and the co-therapeutic agent is clobazam. In some embodiments, the co-therapeutic agent is a combination of stiripentol, valproate and clobazam. In some embodiments of the method, the repeating administration continues over a period of 4 weeks or more until a reduction from baseline in a particular seizure type experienced by the patient is observed. In some embodiments, the repeating administration continues until a particular seizure type experienced by the patient is eliminated for a period of 10 days or more. In some embodiments, the repeating administration continues over a period of 4 weeks or more by administering the fenfluramine twice per day in a liquid formulation in an amount of 0.2 mg/kg/day to 0.8 mg/kg/day until a particular seizure type experienced by the patient is eliminated over a period of 10 days or more. In some aspects, the present disclosure provides a method of treating a patient diagnosed with Dravet syndrome, comprising administering to the patient a therapeutically effective dose of fenfluramine or a pharmaceutically acceptable salt, base or acid thereof in an amount of 0.2 mg/kg/day or more, up to 30 mg/day; administering a co-therapeutic agent; and repeating the administering of the co-therapeutic agent and fenfluramine over a period of weeks until the patient exhibits a reduction from baseline in two types of seizures. In some aspects, provided herein is a use of a formulation for treating a patient diagnosed with Dravet syndrome, wherein the formulation comprises a therapeutically effective dose of fenfluramine or a pharmaceutically acceptable salt, base or acid thereof in an amount of 0.2 mg/kg/day or more, up to 30 mg/day; a co-therapeutic agent; and wherein the co-therapeutic agent and fenfluramine are in a liquid formulation for use over a period of weeks until the patient exhibits a reduction from baseline of two types of seizures. Pharmaceutical compositions and formulations for use in practicing the subject methods are also provided.

The invention includes a use of a formulation for treating a patient diagnosed with Dravet syndrome, wherein the formulation comprises:

a therapeutically effective dose of fenfluramine or a pharmaceutically acceptable salt, base or acid thereof;

a concomitant anti-epileptic drug (AED); and

wherein the fenfluramine and the AED are in a liquid formulation for use in repeated daily administrations over a period of days while gradually reducing AED administered while maintaining efficacy of treatment.

The invention includes a use of a formulation for treating a patient diagnosed with a refractory epilepsy, wherein the formulation comprises:

a therapeutically effective dose of fenfluramine or a pharmaceutically acceptable salt, base or acid thereof in an amount of 0.2 mg/Kg/day or more, up to 30 mg/day;

a concomitant anti-epileptic drug (AED);

monitoring symptoms of the patient;

wherein the fenfluramine and AED are in a liquid formulation for use in repeated daily administrations while gradually reducing AED administered while continuing the monitoring to confirm symptoms are maintained or improved.

The invention includes a use as described throughout, wherein the fenfluramine is the only active ingredient administered to the patient.

The invention includes a use as described throughout, further comprising:

administering a co-therapeutic agent.

The invention includes a use as described throughout, wherein the co-therapeutic agent is selected from the group consisting of, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof.

In some aspects, provided herein is a method of reducing dosage of a concomitant medication in a human patient diagnosed with Dravet syndrome or other epileptic encephalopathy, by administering to the patient a therapeutically effective dose of fenfluramine or a pharmaceutically acceptable salt, base, acid or amine thereof, and repeating the administering over a period of days while reducing the dose of one or more concomitant anti-seizure or anti-epileptic drugs (AEDs) from baseline and thereby decreasing the amount of medication given to the patient while reducing adverse side effects. In some aspects, provided herein is a method of treating a patient diagnosed with Dravet syndrome by administering to the patient a therapeutically effective dose of fenfluramine or a pharmaceutically acceptable salt, base, acid or amine thereof; administering to the patient over a period of days a concomitant AED; and continuing to administer the fenfluramine and the AED over a period of days while gradually reducing AED administered while maintaining the efficacy of treatment. In some embodiments of the method, the concomitant AED is reduced in increments while monitoring efficacy of the treatment. In some embodiments of the method, the incremental reduction continues over a period of days, or over a period of weeks, or over a period of months. In some embodiments of the method, the reduction continues until the patient no longer receives a dose of the concomitant AED. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof. In some embodiments, the administering is over a period of months, and the co-therapeutic agent is clobazam. In some embodiments, the co-therapeutic agent is a combination of stiripentol, valproate and clobazam. In some embodiments, the method further comprises repeating the administration until the clobazam is no longer administered. In some embodiments of the method, the treatment improves two or more symptoms selected from the group consisting of convulsive seizures, ataxias, gait abnormalities, sleep disturbances and cognitive impairment. In some embodiments, the method further comprises repeating the administering of the AED until the amount of AED administered on a daily basis is reduced by 25% or more. In some embodiments, the method further comprises repeating the administering of the AED until the amount of AED administered on a daily basis is reduced by 50% or more. In some embodiments, the method further comprises repeating the administering of the AED until the amount of AED administered on a daily basis is reduced by 75% or more. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some aspects, provided herein is a method of treating a patient diagnosed with refractory epilepsy by administering to the patient over a period of days a therapeutically effective dose of fenfluramine or a pharmaceutically acceptable salt, base or acid thereof in an amount of 0.2 mg/kg/day or more, up to 30 mg/day; administering to the patient over a period of days a concomitant anti-epileptic drug (AED); monitoring symptoms of the patient; and continuing to administer the fenfluramine and AED while gradually reducing AED administered while continuing the monitoring to confirm symptoms are maintained or improved. In some embodiments, the refractory epilepsy is selected from the group consisting of Dravet syndrome, Lennox-Gastaut syndrome, and Doose syndrome. In some embodiments, the refractory epilepsy is Dravet syndrome and the fenfluramine and AED are administered twice daily in a liquid formulation. In some embodiments, the fenfluramine is administered in an amount of 0.2 mg/Kg/day to 0.8 mg/day up to a maximum of 30 mg/day. In some aspects, provided herein is a use of formulation for treating a patient diagnosed with a refractory epilepsy, wherein the formulation comprises a therapeutically effective dose of fenfluramine or a pharmaceutically acceptable salt, base or acid thereof in an amount of 0.2 mg/Kg/day or more, up to 30 mg/day; a concomitant anti-epileptic drug (AED); wherein both the fenfluramine and AED are for use while monitoring symptoms of the patient; and wherein the fenfluramine and AED are used while gradually reducing AED administered while continuing the monitoring to confirm symptoms are maintained or improved. Pharmaceutical compositions and formulations for use in practicing the subject methods are also provided.

The invention includes a use of a formulation for treating a patient in a selected patient population diagnosed with Dravet syndrome, the formulation comprising:

a therapeutically effective dose of fenfluramine or a pharmaceutically acceptable salt, base or acid thereof, and

wherein the formulation is for use with a patient previously determined non-responsive when treated with cannabidiol or the patient's response to cannabidiol diminished over time; and

wherein the use is repeated over a period of days until the patient exhibits a reduction from baseline in convulsive seizure frequency.

The invention includes a use of a formulation for treating a patient in a selected patient population wherein the patient is diagnosed with Dravet syndrome, the formulation comprising:

a therapeutically effective dose of fenfluramine or a pharmaceutically acceptable salt, base or acid thereof in an amount of 0.2 mg/Kg/day or more, up to 30 mg/day;

administering a co-therapeutic agent; and

wherein the formulation is for use with a patient previously determined non-responsive when treated with cannabidiol or the patient's response to cannabidiol diminished over time; and

wherein the use is repeated over a period of weeks until the patient exhibits a reduction from baseline in convulsive seizure frequency of 60% or more.

The invention includes a use as described throughout, wherein the fenfluramine is the only active ingredient administered to the patient.

The invention includes a use as described throughout, further comprising:

administering a co-therapeutic agent.

The invention includes a use as described throughout, wherein the co-therapeutic agent is selected from the group consisting of, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof.

In some aspects, provided herein is a method of treating a selected epileptic patient population, wherein the epileptic patient population is selected based on a determination that the epileptic patients have previously been non-responsive when treated with cannabidiol. In some embodiments, the method comprises selecting the patient based on a previously failed treatment with cannabidiol, based on lack of efficacy or tolerability. The method comprises identifying a patient or a population of patients diagnosed with Dravet syndrome or other epileptic encephalopathy who previously had been non-responsive when treated with cannabidiol or the patient's response to cannabidiol diminished with increasing time. The selected population of patients is then treated by administering, to each identified patient, a therapeutically effective dose of fenfluramine or a pharmaceutically acceptable salt, base, acid or amine thereof; and repeating the administering over a period of a day or days, or over a period of weeks, months or years, until the patient exhibits a reduction from baseline in convulsive seizure frequency. In some aspects, provided herein is a method of treating a patient in a selected patient population diagnosed with Dravet syndrome by determining a patient has previously been non-responsive when treated with cannabidiol or the patient's response to cannabidiol diminished over time; identifying the patient so determined as being non-responsive; administering to the non-responsive patient a therapeutically effective dose of fenfluramine or a pharmaceutically acceptable salt, base or acid thereof; and repeating the administering over a period of days until the patient exhibits a reduction from baseline in convulsive seizure frequency. In some aspects, provided herein is a method of treating a patient in a selected patient population diagnosed with Dravet syndrome by determining a patient has previously been non-responsive when treated with cannabidiol or the patient's response to cannabidiol diminished over time; identifying the patient so determined as being non-responsive; administering to the non-responsive patient a therapeutically effective dose of fenfluramine or a pharmaceutically acceptable salt, base or acid thereof; and repeating the administering over a period of days until the patient exhibits a reduction from baseline in convulsive seizure frequency. In some embodiments of the method, the patient is administered the therapeutically effective dose for a period of weeks/months/years and the reduction from baseline is sustained for a period of weeks/months/years. In some embodiments in which the repeat administration is daily, the administration is once a day, twice a day, three times a day or four times a day. In some embodiments, the dose is provided to the patient at a level of 0.2 mg/kg/day or 0.8 mg/kg/day up to a maximum of 30 mg per day. In some embodiments, the patient exhibits a reduction from baseline in convulsive seizure frequency of 50% or more, 60% or more, 70% or more, 80% or more, 90% or more, 95% or more. In some embodiments, seizures are completely eliminated for 10 days or more, 20 days or more, 30 days or more, 50 days or more, 100 days or more. In some embodiments, the method further comprises repeating the administering until the patient is seizure free for a period of ≥1 day, or for a period of ≥9 days, or for a period of ≥14 days, or for a period of ≥21 days, or for a period of ≥14 weeks, or for a period of ≥6 months, or for a period of ≥1 year. In some embodiments, the method further comprises repeating the administering until the patient is permanently seizure free. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof. In some embodiments, the administering is over a period of months, and the co-therapeutic agent is clobazam. In some embodiments, the co-therapeutic agent is a combination of stiripentol, valproate and clobazam. In some embodiments, the treatment improves two or more symptoms selected from the group consisting of convulsive seizures, ataxias, gait abnormalities, sleep disturbances and cognitive impairment. In some aspects, the present disclosure provides a method of treating a patient in a selected patient population wherein the patient is diagnosed with Dravet syndrome, comprising determining a patient has previously been non-responsive when treated with cannabidiol, or the patient's response to cannabidiol diminished over time; identifying the patient so determined as being non-responsive; administering to the non-responsive patient a therapeutically effective dose of fenfluramine or a pharmaceutically acceptable salt, base or acid thereof in an amount of 0.2 mg/kg/day or more, up to 30 mg/day; administering a co-therapeutic agent; and repeating the administering of the co-therapeutic agent and fenfluramine over a period of weeks until the patient exhibits a reduction from baseline in convulsive seizure frequency of 60% or more. In some aspects, provided herein is a method of adjusting dose of cannabidiol in a human patient diagnosed with Dravet syndrome or other epileptic encephalopathy, by administering, to a patient receiving cannabidiol, a therapeutically effective dose of fenfluramine or a pharmaceutically acceptable salt, base, acid or amine thereof, and increasing the fenfluramine dosage to 0.4 mg/kg/day for days 18-24 of fenfluramine therapy; and thereafter increasing the daily dosage to 0.5 mg/kg/day; provided that the total dosage of fenfluramine does not exceed 20 mg/day. In some aspects, provided herein is a method of dosing a patient with fenfluramine, wherein the patient is receiving cannabidiol therapy and commencing fenfluramine therapy for treating a form of epilepsy, by administering to the patient receiving cannabidiol an initial dosage of fenfluramine of 0.2 mg/kg/day for the first seven days of fenfluramine therapy; increasing the initial dosage to 0.4 mg/kg/day for days 18-24 of fenfluramine therapy; and thereafter increasing the daily dosage to 0.5 mg/kg/day; provided that the total dosage of fenfluramine does not exceed 20 mg/day. If a treating physician determines that a patient needs a more rapid titration the dose may be increased in increments of not more than 0.2 mg/kg/day every 4 days, up to a dose of 0.5 mg/kg/day or a maximum dose of 20 mg/day. In some embodiments of these methods, the form of epilepsy is chosen from Dravet syndrome, Lennox-Gastaut syndrome and Doose syndrome. In some embodiments of these methods, the titration provides increased tolerability of the combination of cannabidiol and fenfluramine. In some embodiments, the patient is already receiving one or more co-therapeutic agents in addition to cannabidiol. In some aspects, the present disclosure provides a method of treating a patient in a selected patient population wherein the patient is diagnosed with Dravet syndrome, comprising determining a patient has previously been non-responsive when treated with cannabidiol or the patient's response to cannabidiol diminished over time; identifying the patient so determined as being non-responsive; administering to the non-responsive patient a therapeutically effective dose of fenfluramine or a pharmaceutically acceptable salt, base or acid thereof in an amount of 0.2 mg/kg/day or more, up to 30 mg/day; administering a co-therapeutic agent; and repeating the administering of the co-therapeutic agent and fenfluramine over a period of weeks until the patient exhibits a reduction from baseline in convulsive seizure frequency of 60% or more. In some aspects, provided herein is a use of a formulation for treating a patient in a selected patient population wherein the patient is diagnosed with Dravet syndrome, wherein the formulation comprises a therapeutically effective dose of fenfluramine or a pharmaceutically acceptable salt, base or acid thereof in an amount of 0.2 mg/kg/day or more, up to 30 mg/day; a co-therapeutic agent; and wherein the formulation is used with a patient previously determined to be non-responsive when treated with cannabidiol, or the patient's response to cannabidiol diminished over time; wherein the co-therapeutic agent and fenfluramine are for use over a period of weeks until the patient is determined as non-responsive to cannabidiol exhibits a reduction from baseline in convulsive seizure frequency of 60% or more. Pharmaceutical compositions and formulations for use in practicing the subject methods are also provided.

The invention includes a use of a formulation for treating a patient in a selected patient population diagnosed with Dravet syndrome, the formulation comprising:

a therapeutically effective dose of fenfluramine or a pharmaceutically acceptable salt, base or acid thereof;

wherein the formulation is for use with a patient previously determine non-responsive when treated with stiripentol or the patient's response to stiripentol diminished over time; and

wherein the use is repeated over a period of days until the patient exhibits a reduction from baseline in convulsive seizure frequency.

The invention includes a use of a formulation for treating a patient in a selected patient population wherein the patient is diagnosed with Dravet syndrome, the use comprising:

a therapeutically effective dose of fenfluramine or a pharmaceutically acceptable salt, base or acid thereof in an amount of 0.2 mg/Kg/day or more, up to 30 mg/day;

administering a co-therapeutic agent; and

wherein the formulation is for use with a patient previously determined non-responsive when treated with stiripentol or the patient's response to stiripentol diminished over time;

wherein the use is repeated over a period of weeks until the patient exhibits a reduction from baseline in convulsive seizure frequency of 60% or more.

The invention includes a use as described throughout, wherein the fenfluramine is the only active ingredient administered to the patient.

The invention includes a use as described throughout, further comprising:

administering a co-therapeutic agent.

The invention includes a use as described throughout, wherein the co-therapeutic agent is selected from the group consisting of, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof.

In some aspects, provided herein is a method of treating a selected epileptic patient population, wherein the epileptic patient population is selected based on a determination that the epileptic patients have previously been non-responsive when treated with stiripentol. In some embodiments, the method comprises selecting the patient based on a previously failed treatment with stiripentol, based on lack of efficacy or tolerability. The method comprises identifying a patient or a population of patients diagnosed with Dravet syndrome or other epileptic encephalopathy who previously had been non-responsive when treated with stiripentol or the patient's response to stiripentol diminished with increasing time. The selected population of patients is then treated by administering, to each identified patient, a therapeutically effective dose of fenfluramine or a pharmaceutically acceptable salt, base, acid or amine thereof; and repeating the administering over a period of a day or days, or over a period of weeks, months or years, until the patient exhibits a reduction from baseline in convulsive seizure frequency. In some aspects, provided herein is a method of treating a patient in a selected patient population diagnosed with Dravet syndrome by determining a patient has previously been non-responsive when treated with stiripentol or the patient's response to stiripentol diminished over time; identifying the patient so determined as being non-responsive; administering to the non-responsive patient a therapeutically effective dose of fenfluramine or a pharmaceutically acceptable salt, base or acid thereof; and repeating the administering over a period of days until the patient exhibits a reduction from baseline in convulsive seizure frequency. In some embodiments of the method, the patient is administered the therapeutically effective dose for a period of weeks/months/years and the reduction from baseline is sustained for a period of weeks/months/years. In some embodiments in which the repeat administration is daily, the administration is once a day, twice a day, three times a day or four times a day. In some embodiments, the dose is provided to the patient at a level of 0.2 mg/kg/day or 0.8 mg/kg/day up to a maximum of 30 mg per day. In some embodiments, the patient exhibits a reduction from baseline in convulsive seizure frequency of 50% or more, 60% or more, 70% or more, 80% or more, 90% or more, 95% or more. In some embodiments, seizures are completely eliminated for 10 days or more, 20 days or more, 30 days or more, 50 days or more, 100 days or more. In some embodiments, the method further comprises repeating the administering until the patient is seizure free for a period of ≥1 day, or for a period of ≥9 days, or for a period of ≥14 days, or for a period of ≥21 days, or for a period of ≥14 weeks, or for a period of ≥6 months, or for a period of ≥1 year. In some embodiments, the method further comprises repeating the administering until the patient is permanently seizure free. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof. In some embodiments, the administering is over a period of months, and the co-therapeutic agent is clobazam. In some embodiments, the co-therapeutic agent is a combination of stiripentol, valproate and clobazam. In some embodiments, the treatment improves two or more symptoms selected from the group consisting of convulsive seizures, ataxias, gait abnormalities, sleep disturbances and cognitive impairment. In some aspects, the present disclosure provides a method of treating a patient in a selected patient population wherein the patient is diagnosed with Dravet syndrome, comprising determining a patient has previously been non-responsive when treated with stiripentol or the patient's response to stiripentol diminished over time; identifying the patient so determined as being non-responsive; administering to the non-responsive patient a therapeutically effective dose of fenfluramine or a pharmaceutically acceptable salt, base or acid thereof in an amount of 0.2 mg/kg/day or more, up to 30 mg/day; administering a co-therapeutic agent; and repeating the administering of the co-therapeutic agent and fenfluramine over a period of weeks until the patient exhibits a reduction from baseline in convulsive seizure frequency of 60% or more. In some aspects, provided herein is a method of adjusting dose of stiripentol in a human patient diagnosed with Dravet syndrome or other epileptic encephalopathy, by administering, to a patient receiving stiripentol, a therapeutically effective dose of fenfluramine or a pharmaceutically acceptable salt, base, acid or amine thereof, and increasing the fenfluramine dosage to 0.4 mg/kg/day for days 18-24 of fenfluramine therapy; and thereafter increasing the daily dosage to 0.5 mg/kg/day; provided that the total dosage of fenfluramine does not exceed 20 mg/day. In some aspects, provided herein is a method of dosing a patient with fenfluramine, wherein the patient is receiving stiripentol therapy and commencing fenfluramine therapy for treating a form of epilepsy, by administering to the patient receiving stiripentol an initial dosage of fenfluramine of 0.2 mg/kg/day for the first seven days of fenfluramine therapy; increasing the initial dosage to 0.4 mg/kg/day for days 18-24 of fenfluramine therapy; and thereafter increasing the daily dosage to 0.5 mg/kg/day; provided that the total dosage of fenfluramine does not exceed 20 mg/day. If a treating physician determines that a patient needs a more rapid titration the dose may be increased in increments of not more than 0.2 mg/kg/day every 4 days, up to a dose of 0.5 mg/kg/day or a maximum dose of 20 mg/day. In some embodiments of these methods, the form of epilepsy is chosen from Dravet syndrome, Lennox-Gastaut syndrome and Doose syndrome. In some embodiments of these methods, the titration provides increased tolerability of the combination of stiripentol and fenfluramine. In some embodiments, the patient is already receiving one or more co-therapeutic agents in addition to stiripentol. In some aspects, provided herein is a use of a formulation for treating a patient in a selected patient population wherein the patient is diagnosed with Dravet syndrome, wherein the formulation comprises a therapeutically effective dose of fenfluramine or a pharmaceutically acceptable salt, base or acid thereof in an amount of 0.2 mg/kg/day or more, up to 30 mg/day; a co-therapeutic agent; and wherein the formulation is used with a patient previously determined to be non-responsive when treated with stiripentol, or the patient's response to stiripentol diminished over time; wherein the co-therapeutic agent and fenfluramine are for use over a period of weeks until the patient is determined as non-responsive to stiripentol exhibits a reduction from baseline in convulsive seizure frequency of 60% or more. Pharmaceutical compositions and formulations for use in practicing the subject methods are also provided.

The formulation may include flavoring and coloring agents or may be completely devoid of any excipient materials beyond those necessary to dissolve the fenfluramine in the liquid which may be water.

In some embodiments of the method, fenfluramine is the only active ingredient administered to the patient. In some embodiments, the method further comprises administering a co-therapeutic agent. In some embodiments, the fenfluramine is adjunctive therapy and is co-administered with a second, or a second and third, or a second, third and fourth, therapeutic agent. Any second, or second and third, or second, third and fourth therapeutic agents may be utilized. In some cases, the additional therapeutic agents are selected from the group consisting of cannabidiol, carbamazepine, ethosuximide, fosphenytoin, lamotrigine, levetiracetam, phenobarbital, topiramate, stiripentol, valproic acid, valproate, verapamil, and benzodiazepines such as clobazam, clonazepam, diazepam, lorazepam, and midazolam and a pharmaceutically acceptable salt or base thereof.

Aspects of the subject methods include identifying a patient previously treated unsuccessfully with stiripentol who will benefit from treatment with fenfluramine according to the methods described herein. Fenfluramine can then be employed to treat the patient either as a subsequent monotherapy or as a co-therapy with stiripentol. In some cases, the patient can be monitored for a reduction in instances of seizures (e.g., mean monthly convulsive seizures) relative to that observed under prior treatment with stiripentol.

Fenfluramine can be employed to treat a patient who has previously been treated with cannabidiol. In some instances, the patient is diagnosed with Dravet syndrome that is refractory to treatment with cannabidiol. By refractory to cannabidiol is meant that the frequency of convulsive seizures (CSF) is not significantly reduced in the patient in response to therapy (e.g., monotherapy) with cannabidiol (CBD). In some cases, a significant reduction in CSF is a 10% or greater reduction in mean monthly convulsive seizures, such as 15% or greater, 20% or greater, 25% or greater, 30% or greater, 35% or greater, 40% or greater, or 45% or greater reduction. In certain instances, the subject method is a method of preventing or treating seizures in a patient diagnosed with Dravet syndrome refractory to treatment with cannabidiol by administering to that patient a therapeutically effective dose of fenfluramine, whereby seizures are prevented or reduced. In various embodiments of this aspect, the instances of seizures (e.g., mean monthly convulsive seizures) are decreased by at least 50%, at least 60%, at least 70%, at least 80% or at least 90%. Aspects of the subject methods include identifying a patient previously treated unsuccessfully with cannabidiol who will benefit from treatment with fenfluramine according to the methods described herein.

Fenfluramine can then be employed to treat the patient either as a subsequent monotherapy or as a co-therapy with a second agent, such as cannabidiol. In some cases, the patient can be monitored for a reduction in instances of seizures (e.g., mean monthly convulsive seizures) relative to that observed under prior treatment with cannabidiol.

Fenfluramine can be administered in the form of the free base, or in the form of a pharmaceutically acceptable salt, for example selected from the group consisting of hydrochloride, hydrobromide, hydroiodide, maleate, sulphate, tartrate, acetate, citrate, tosylate, succinate, mesylate and besylate. Further illustrative pharmaceutically acceptable salts can be found in Berge et al., J. Pharm. Sci. (1977) 68(1):1-19.

Fenfluramine for use in the methods of the present invention may be produced according to any pharmaceutically acceptable process known to those skilled in the art.

Examples of processes for synthesizing fenfluramine are provided in the following documents: GB1413070, GB1413078 and EP441160. An example of a fenfluramine drug product synthesis is provided in US20180148403.

The dose of fenfluramine to be used in a method of the present invention can be provided in the form of a kit, including instructions for using the dose in one or more of the methods of the present invention. In certain embodiments, the kit can additionally comprise a dosage form comprising one or more co-therapeutic agents. The kit may also contain directions for initiating fenfluramine therapy in a patient, in some instances the direction may take into account co-administration with other interacting antiepileptic drugs and provide alternate dosing instructions when the patient also receives those drugs concomitantly.

A method of the present invention can be practiced on any appropriately diagnosed patient. In a typical embodiment of the present invention, the patient may be an adult, and may be aged about 18 or less, about 16 or less, about 14 or less, about 12 or less, about 10 or less, about 8 or less, about 6 or less or about 4 or less to about 0 months or more, about 1 month or more, about 2 months or more, about 4 months or more, about 6 months or more or about 1 year or more. Thus, the diagnosed patient is typically about one month old or older when treated.

The invention is further illustrated in the following Comparative Examples.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Table 1 provides results based on the data presented in Ceulemans et al., Epilepsia (2012) 53(7):1131-1139. Patients were administered an average daily dose of fenfluramine of 0.34 mg/kg/day for between 1 and 22 years.

TABLE 1
Seizure Free Patients and Responders
(Treated with Fenfluramine and Valproate)
Fenfluramine

| Seizure-free Patients | >50% Reduction in Seizures |
|---|---|
| 8/12 (66%) | 9/12 (75%) |

As can be seen from the foregoing data, long-term fenfluramine treatment advantageously resulted in a seizure-free condition in 66.6% of test subjects.

Additionally, long-term fenfluramine treatment advantageously resulted in a reduction in seizures of 75%.

These results confirm that fenfluramine provides long term elimination/reduction in seizures.

These results were achieved, in the vast number of cases, using significantly lower doses of fenfluramine than those proposed previously in the treatment of various conditions typified by seizures. Additionally, and surprisingly, fenfluramine effectively reduced the incidence of all types of seizures and not only photosensitive or self-induced seizures.

The subjects treated with fenfluramine were monitored using echocardiography for possible heart valve defects. No clinically relevant defects were identified.

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

### Example 2

Two identical Phase 3 studies were carried out with a liquid formulation of fenfluramine. Approximately 120 subjects with Dravet syndrome, a rare form of pediatric epilepsy, were intended to be randomized in each study in three treatment groups: 0.2 mg/kg/day of the liquid fenfluramine formulation, 0.8 mg/kg/day the liquid fenfluramine formulation and placebo (n=40/group).

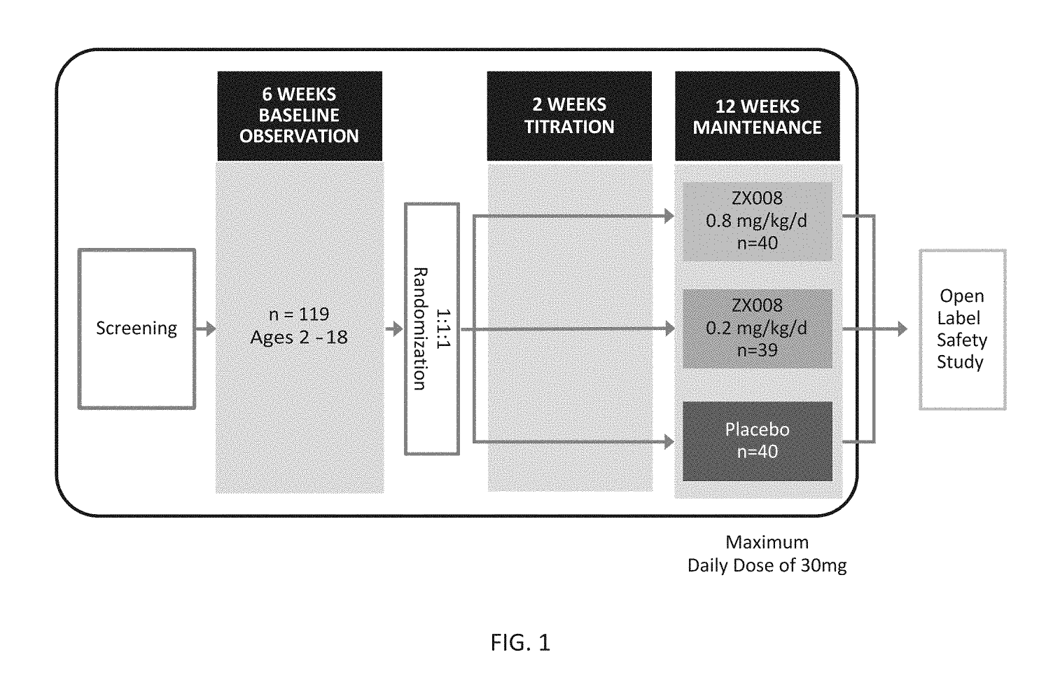

The first 119 subjects to be randomized in both identical studies combined were analyzed and reported as Study 1 (FIG. 1), covered by a Statistical Analysis Plan (SAP).

Primary and key secondary endpoints in the Study 1 SAP were pre-determined. All patients were treated twice per day over a period of 14 weeks using an oral syringe to administer the formulation. The treatment period included a two-week titration period and a 12-week maintenance period.

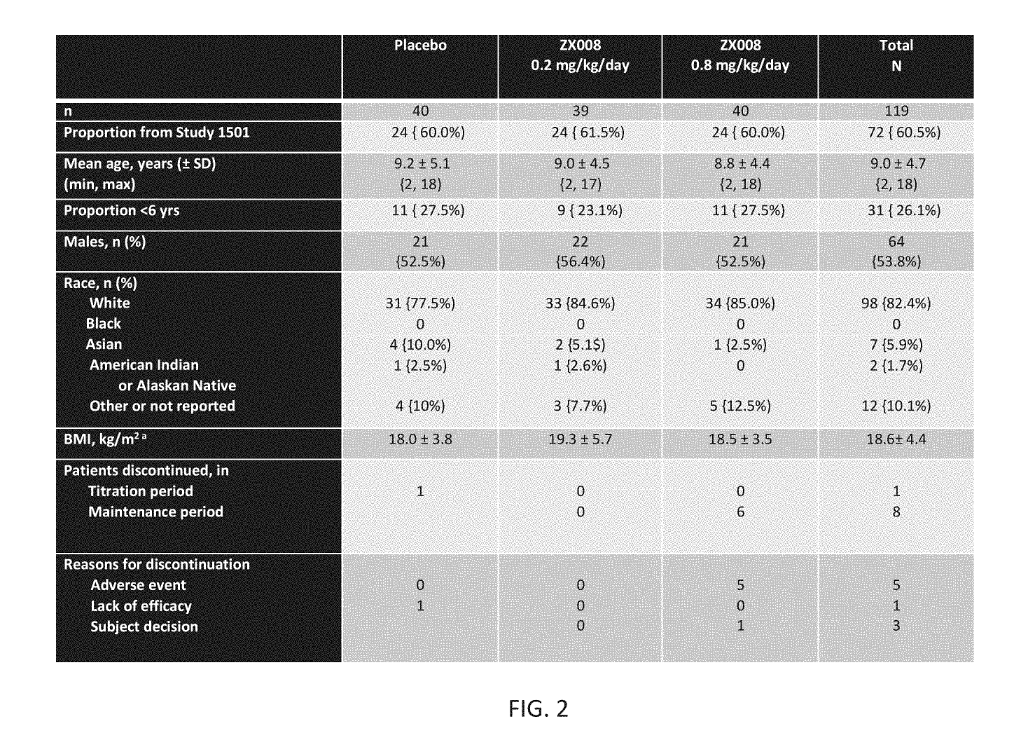

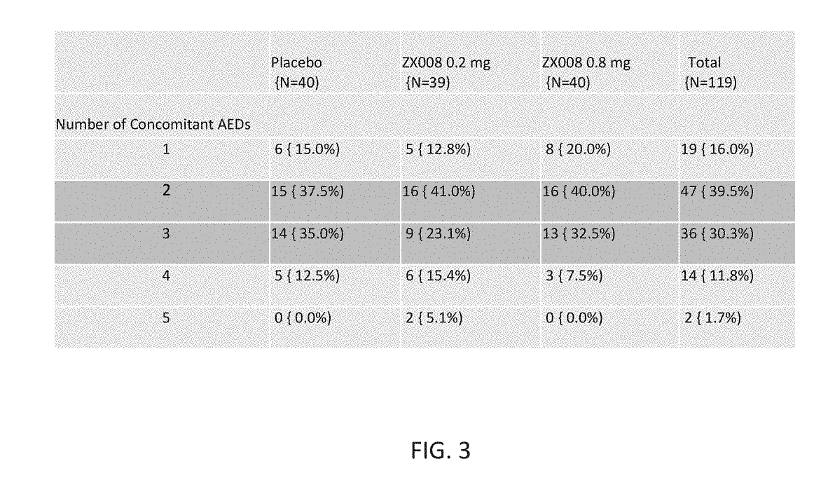

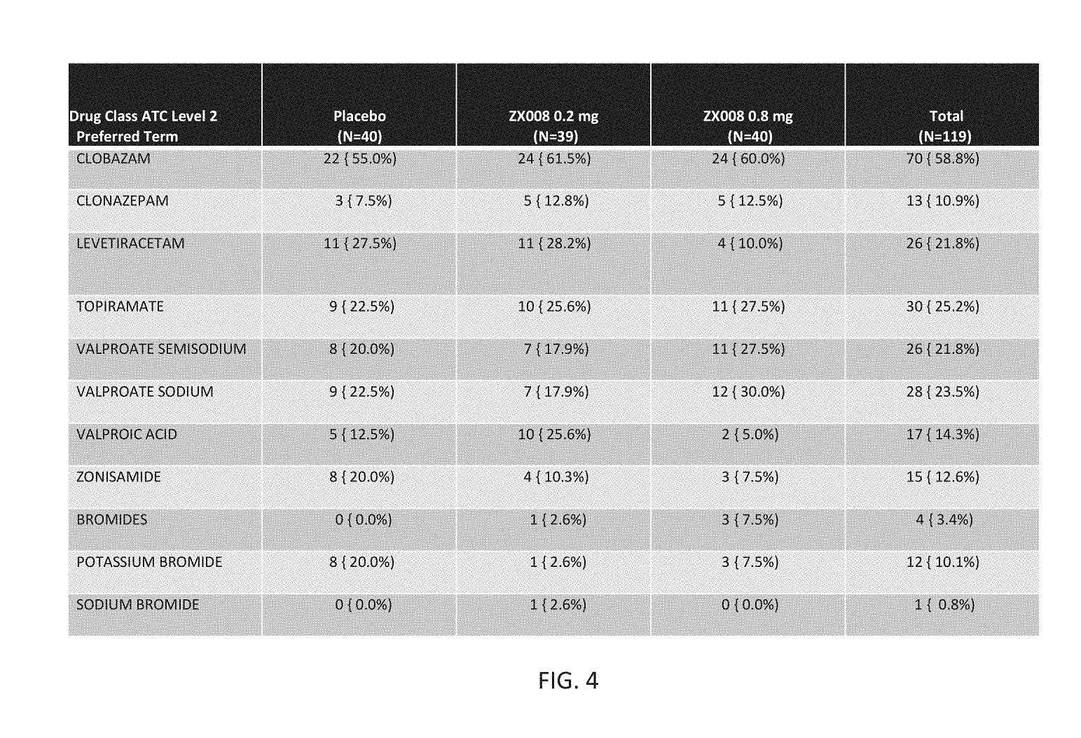

The initial results of Study 1 are outlined below. The randomized, double blind, placebo controlled, Phase 3 study enrolled 119 patients across sites in the United States, Canada, Europe and Australia. The median age of patients was 8 years, the mean age of patients was 9 years (range, 2-18 years) (FIG. 2).

Following a six-week baseline observation period, patients were randomized to one of three treatment groups: liquid fenfluramine formulation 0.8 mg/kg/day (30 mg maximum daily dose; n=40), liquid fenfluramine formulation 0.2 mg/kg/day (n=39) and placebo (n=40) in which liquid fenfluramine formulation or placebo was added to current regimens of antiepileptic drugs. Patients were titrated to their target dose over two weeks, and then remained at that fixed dose for 12 weeks. The mean baseline convulsive seizure frequency across the study groups was approximately 40 seizures per month. 110 (92%) patients completed the study (85% 0.8 mg/kg/day; 100% 0.2 mg/kg/day; 93% placebo).

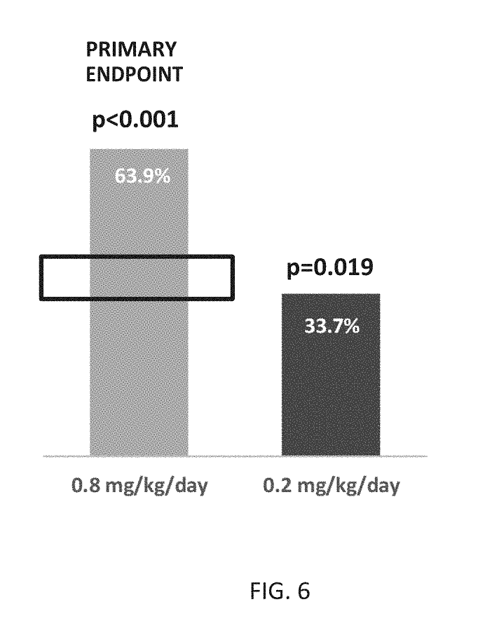

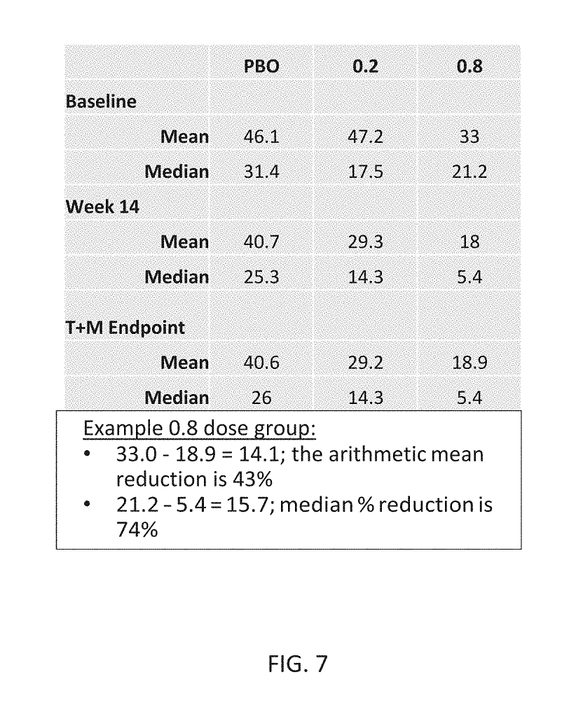

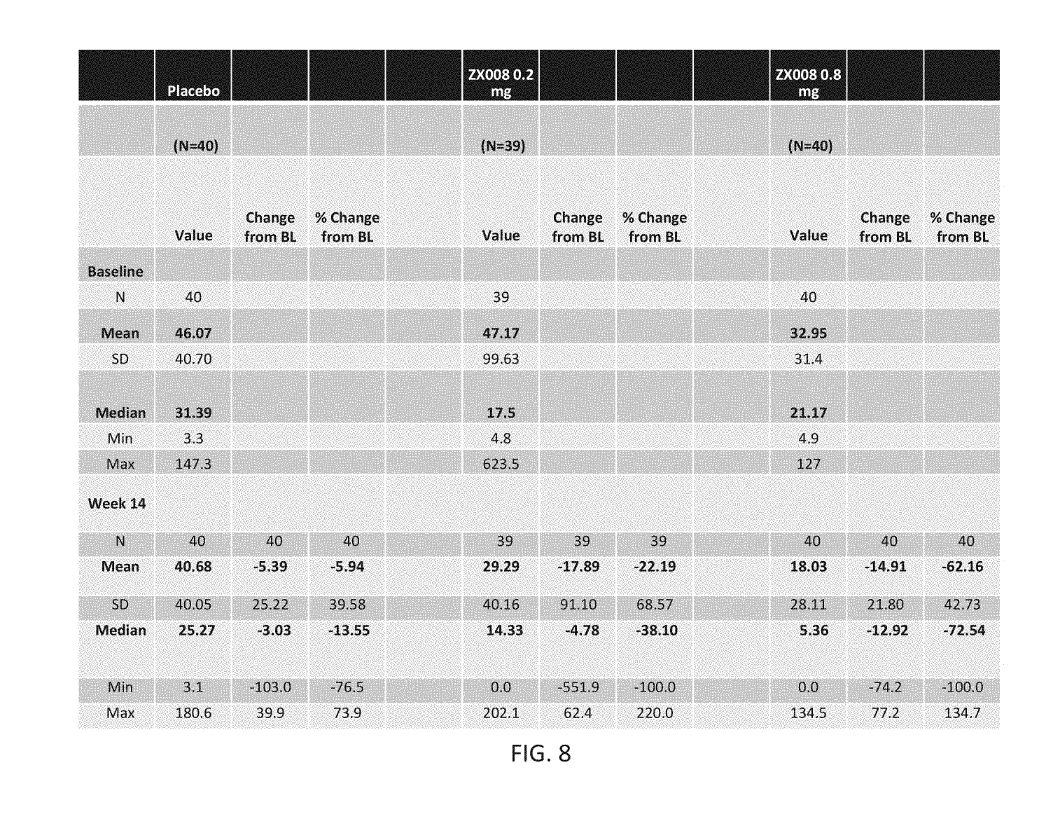

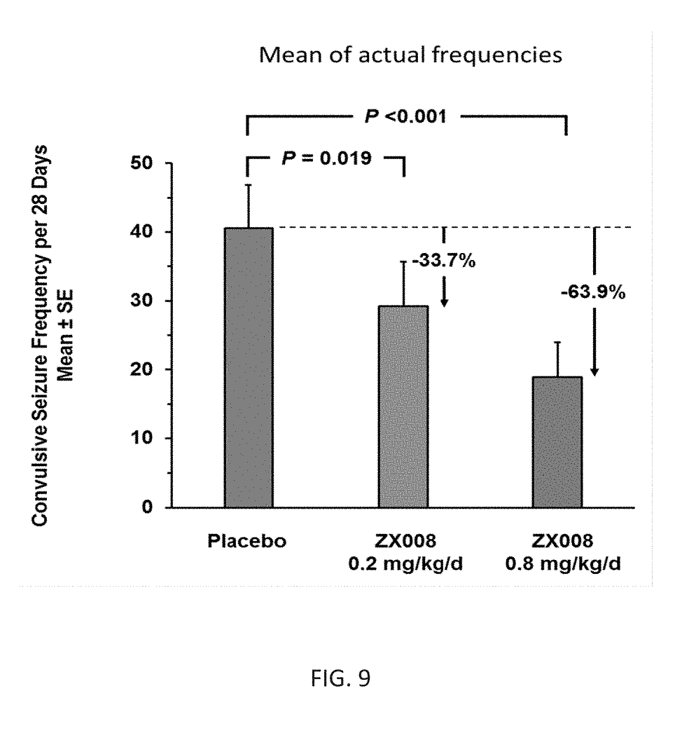

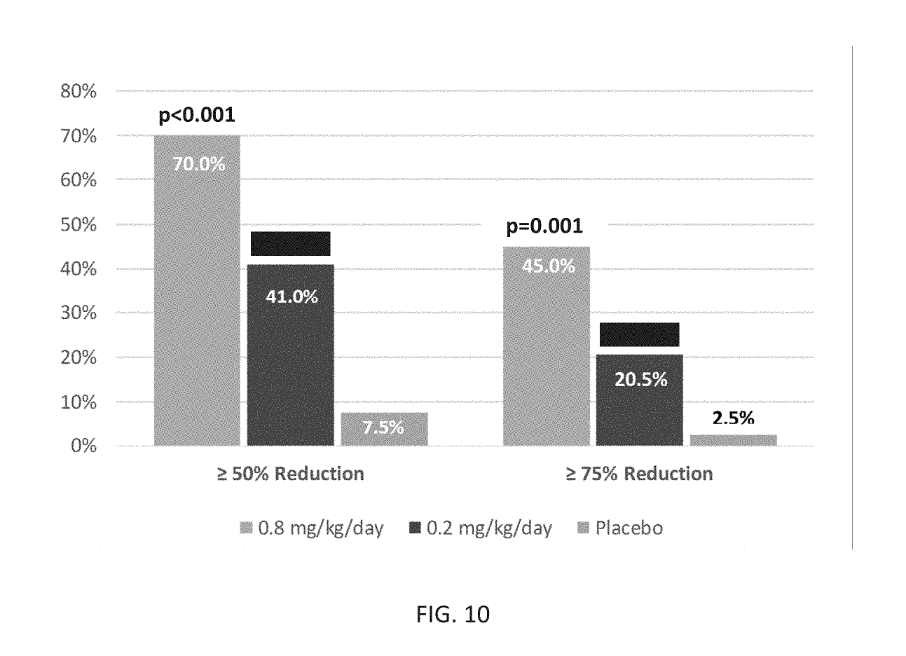

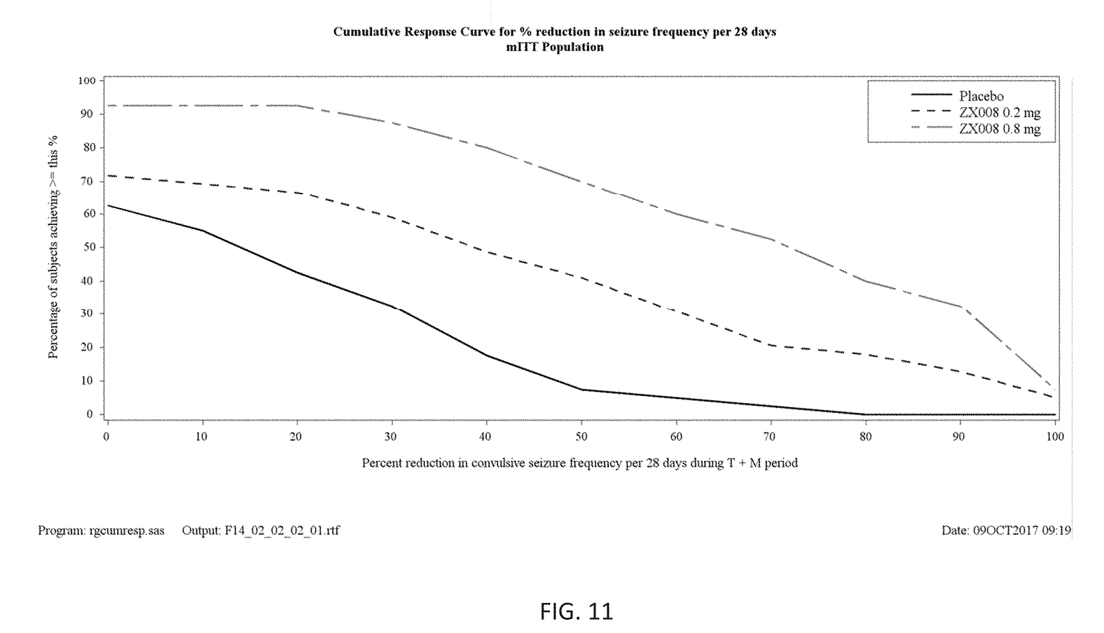

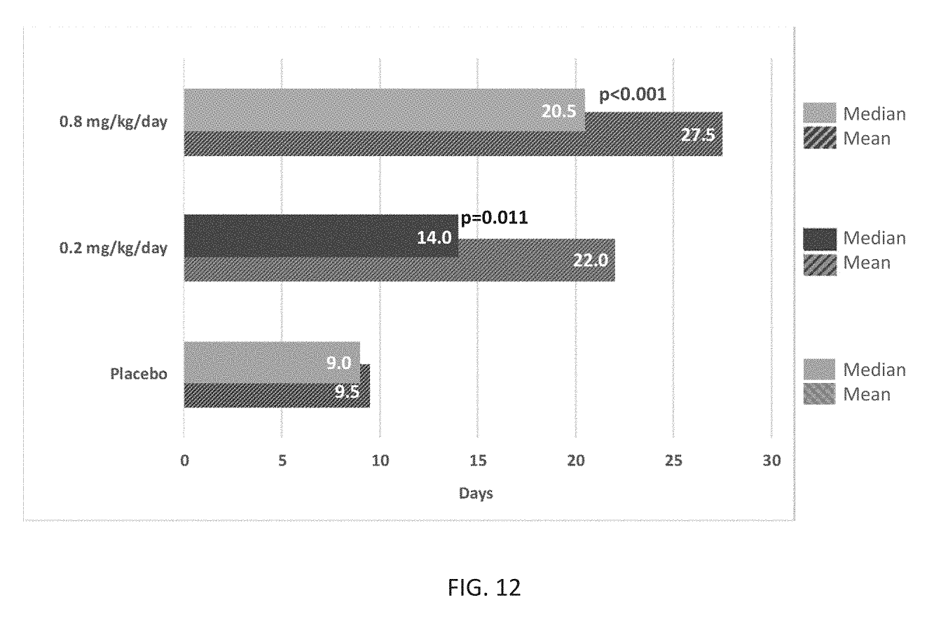

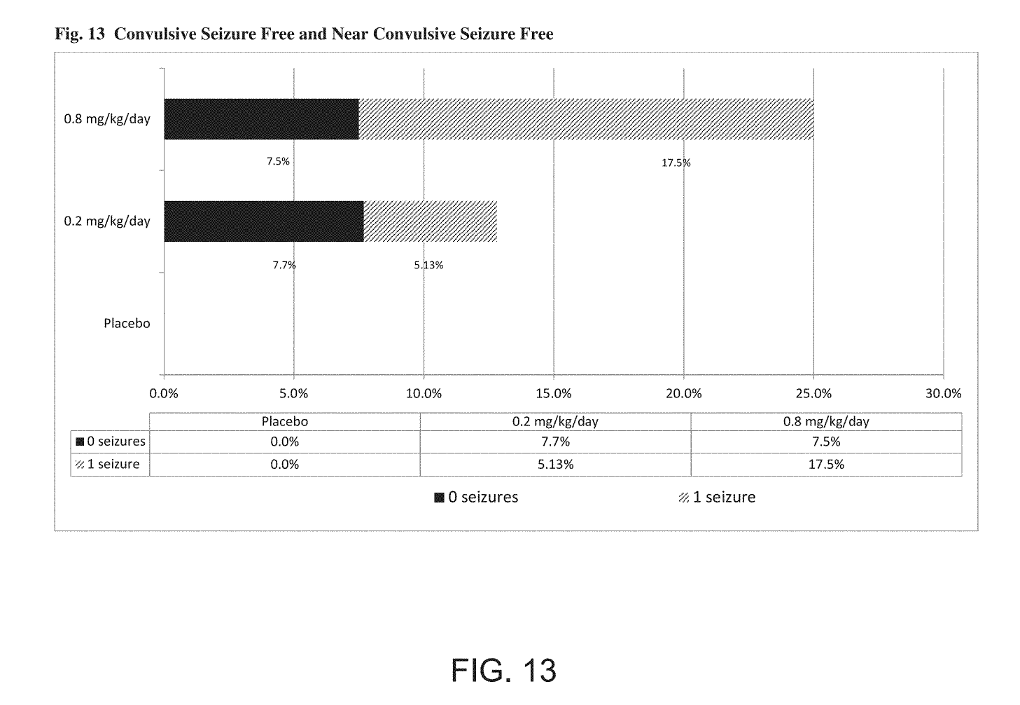

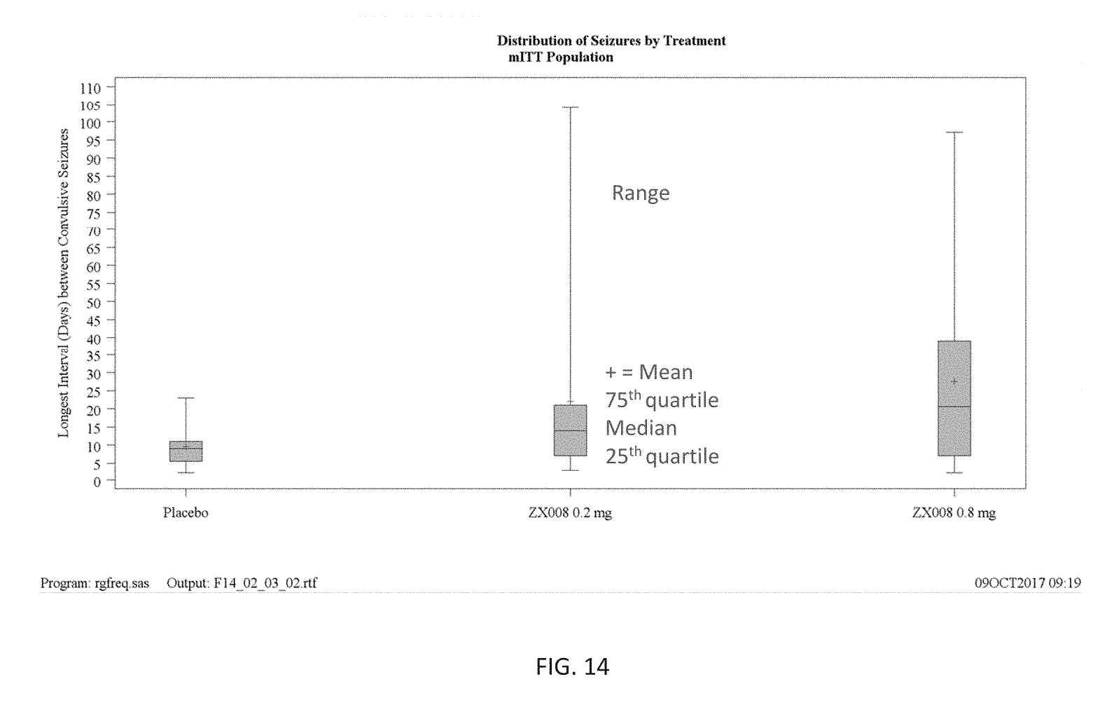

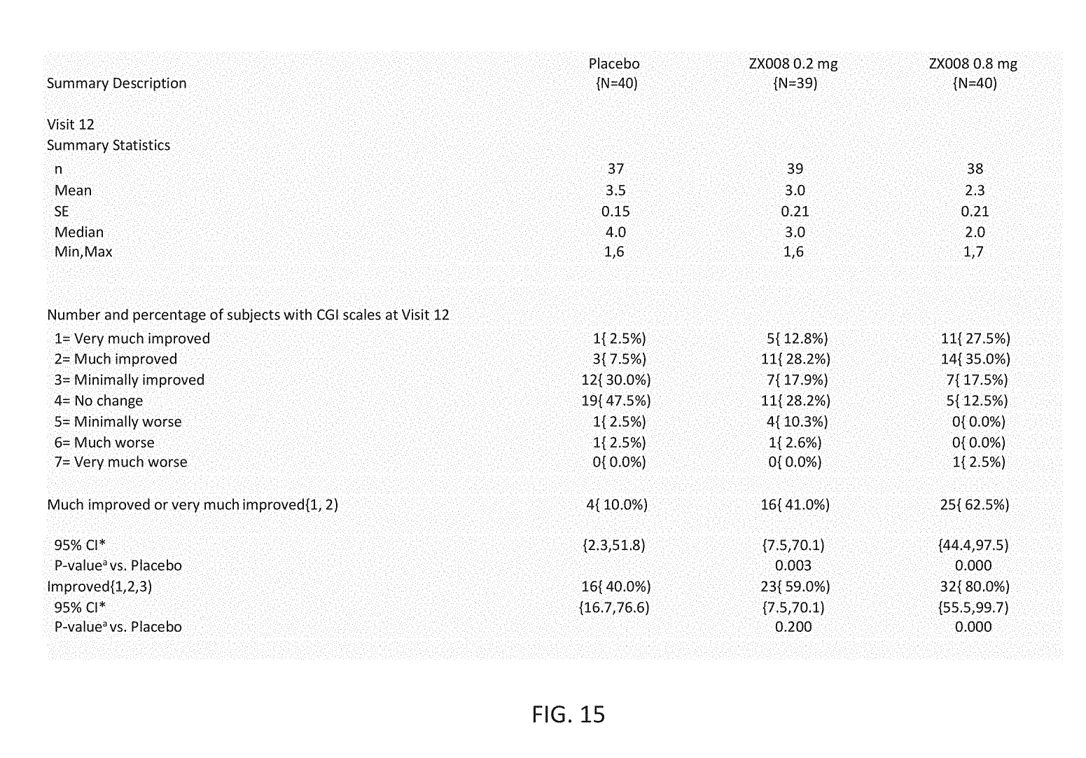

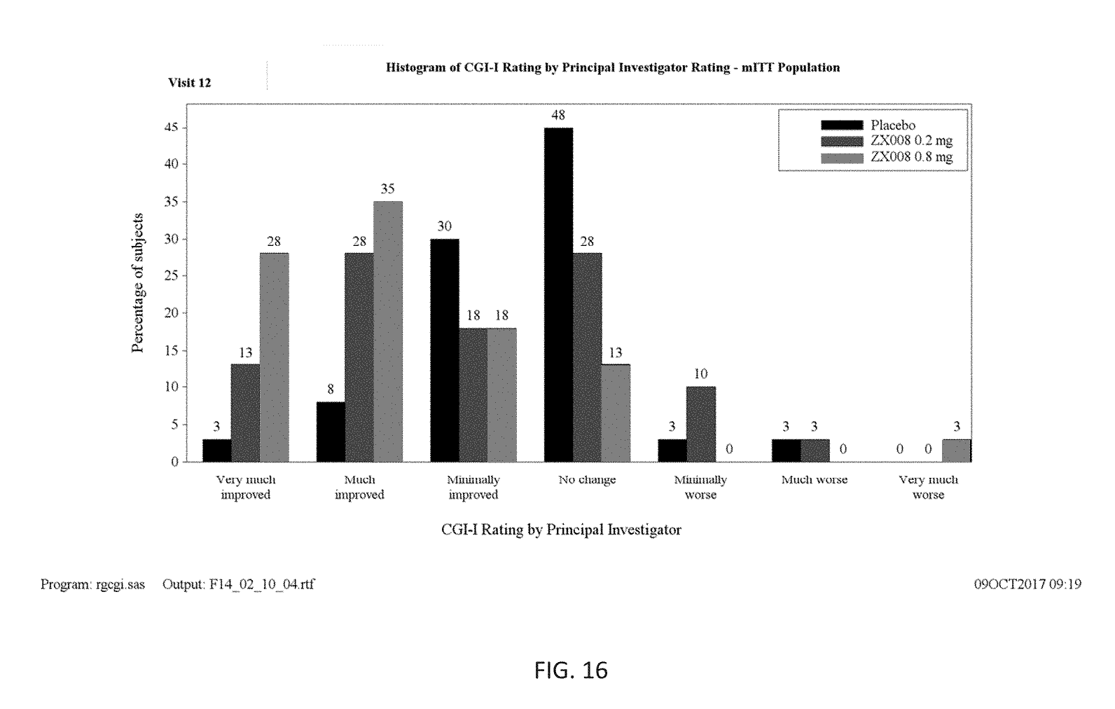

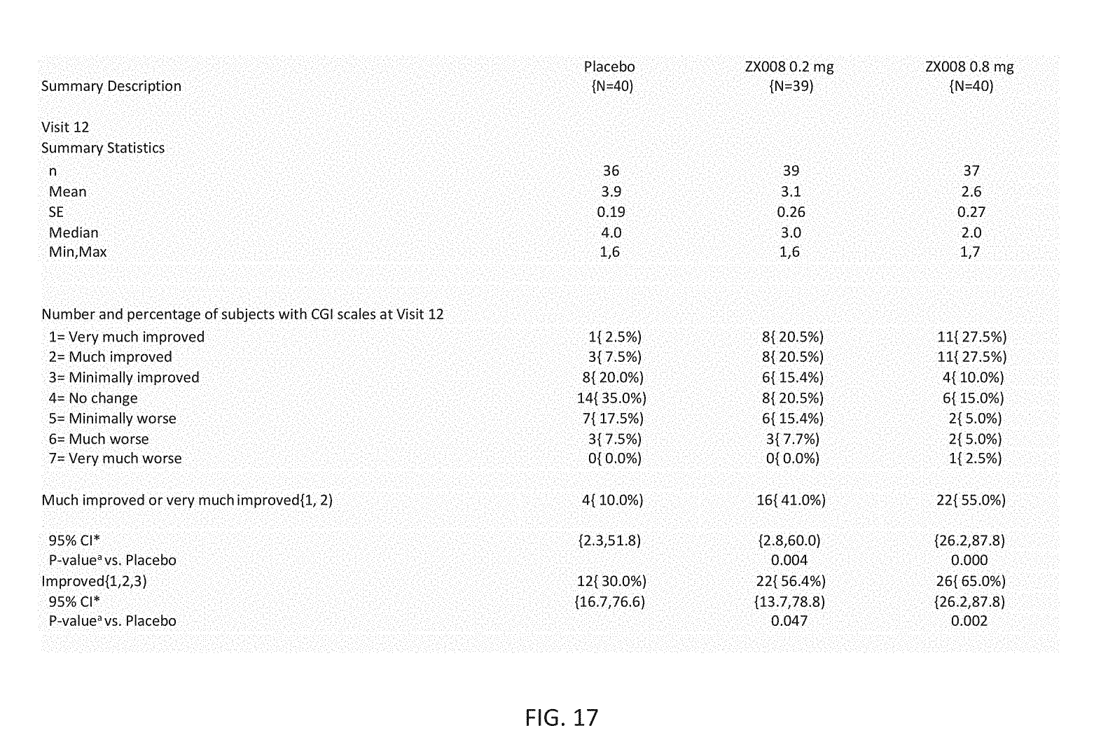

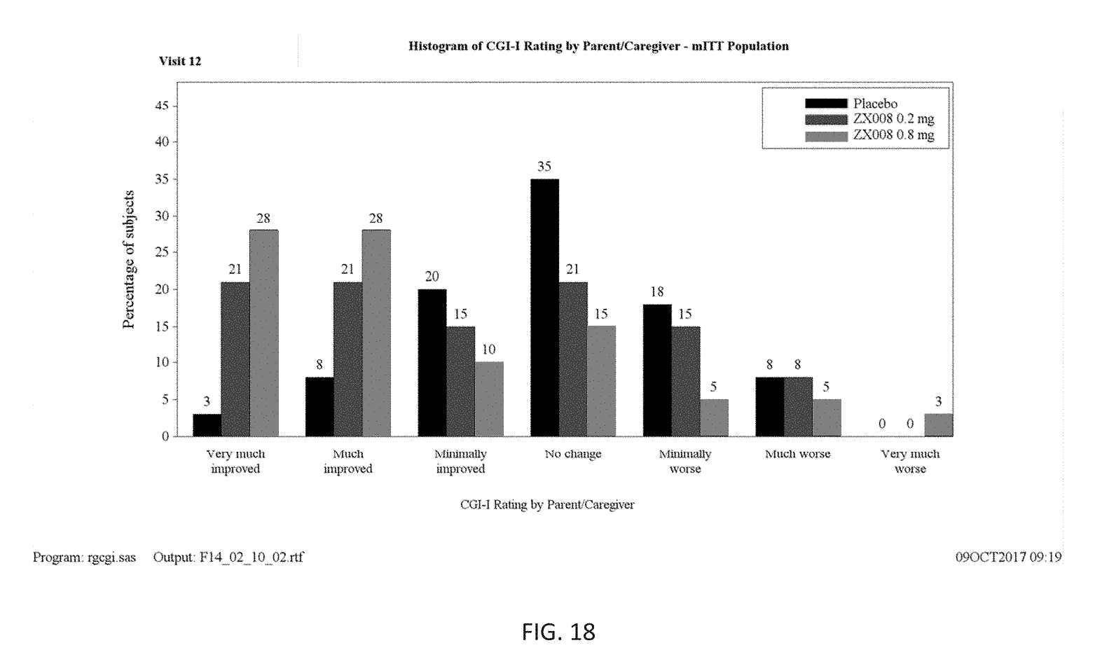

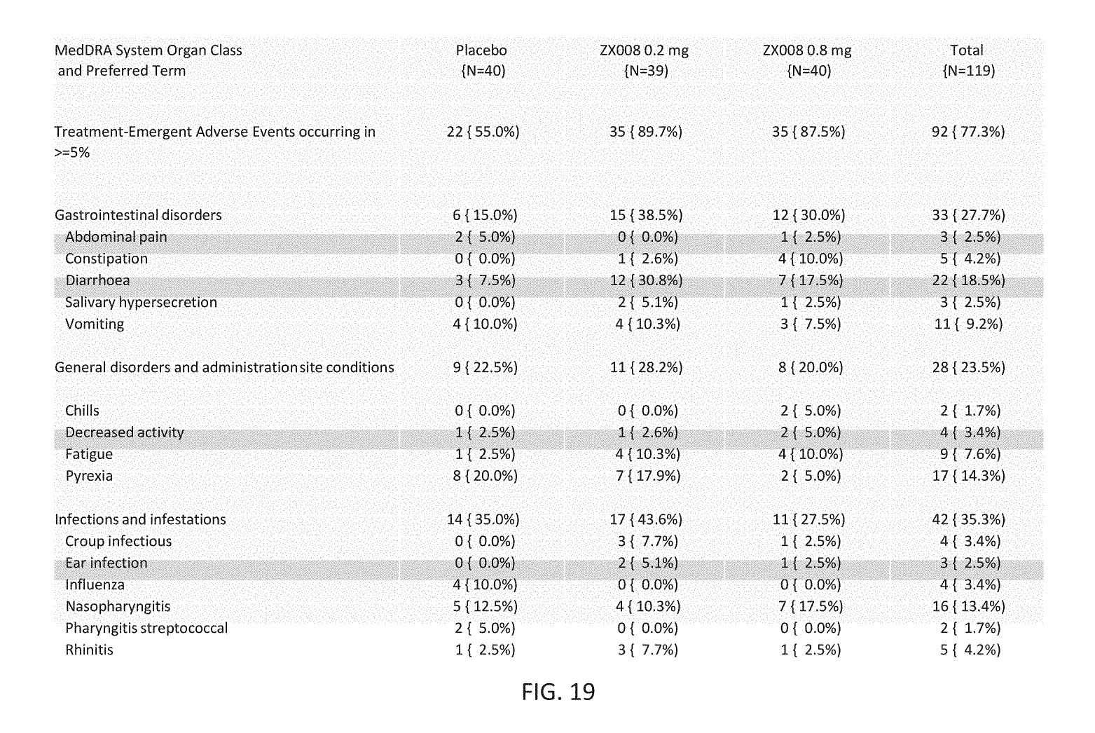

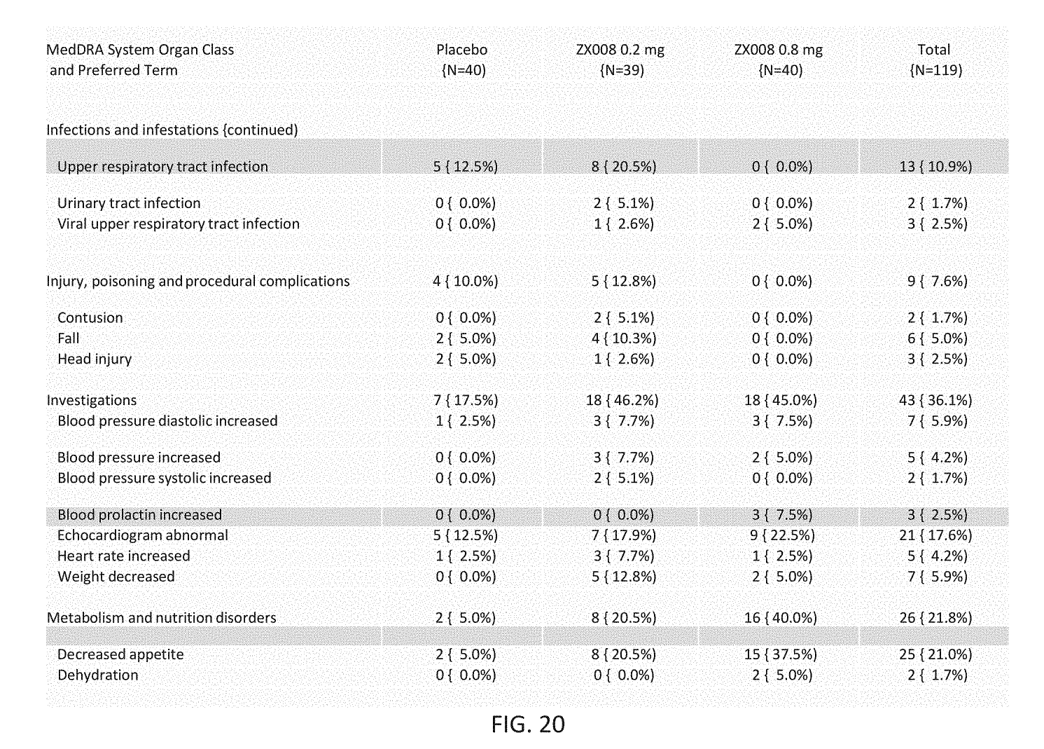

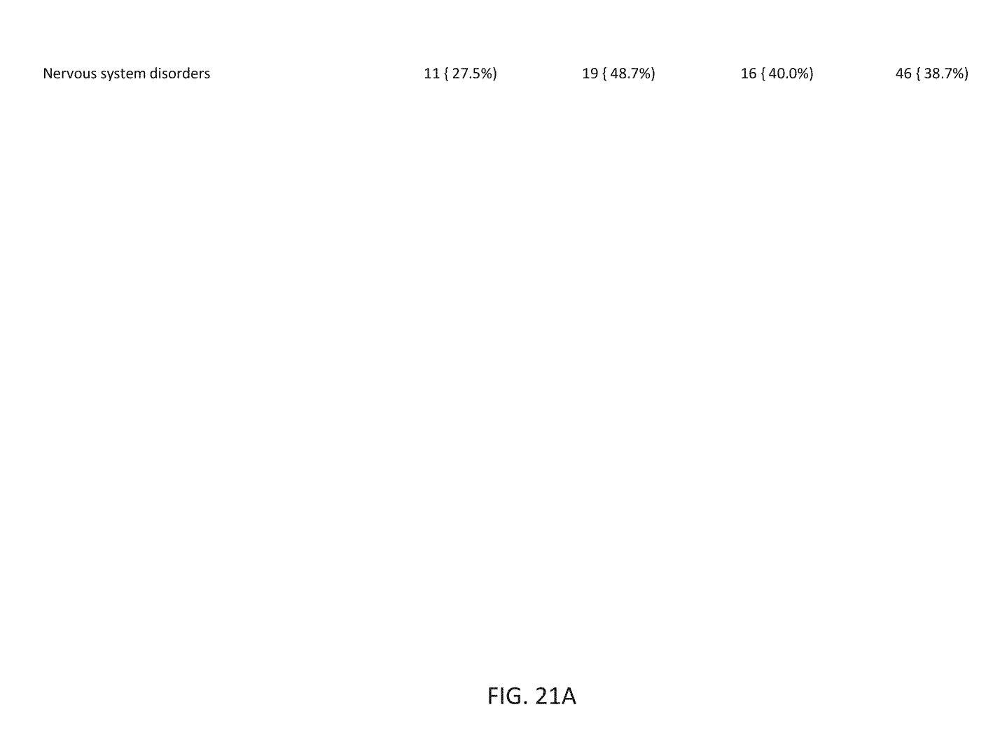

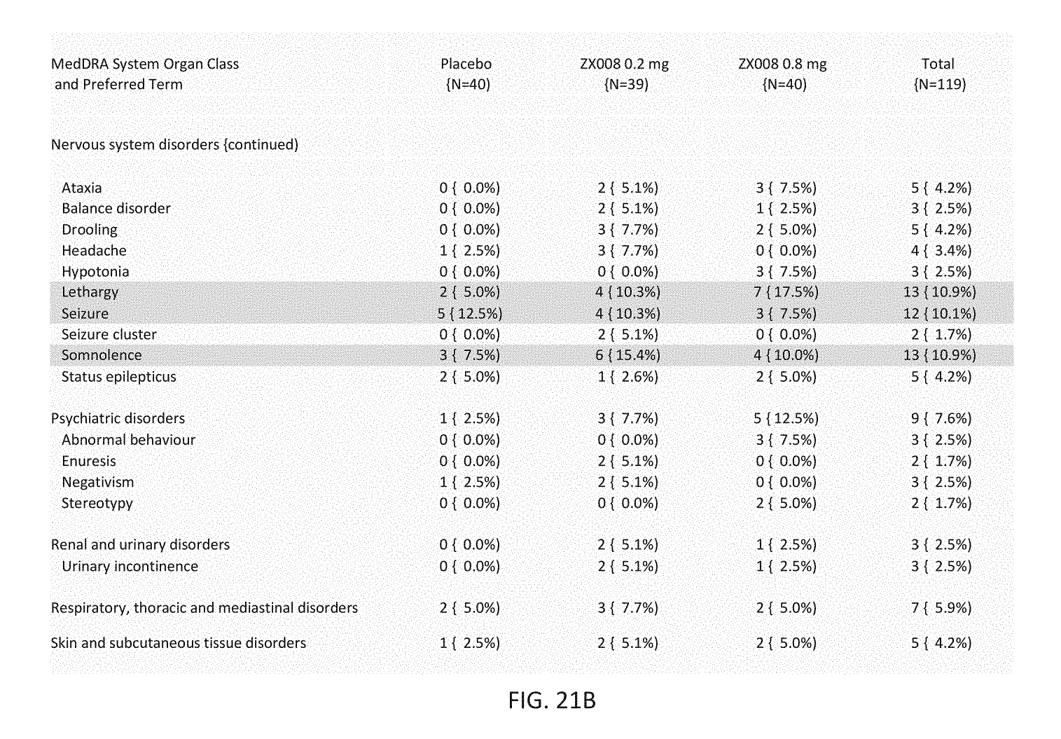

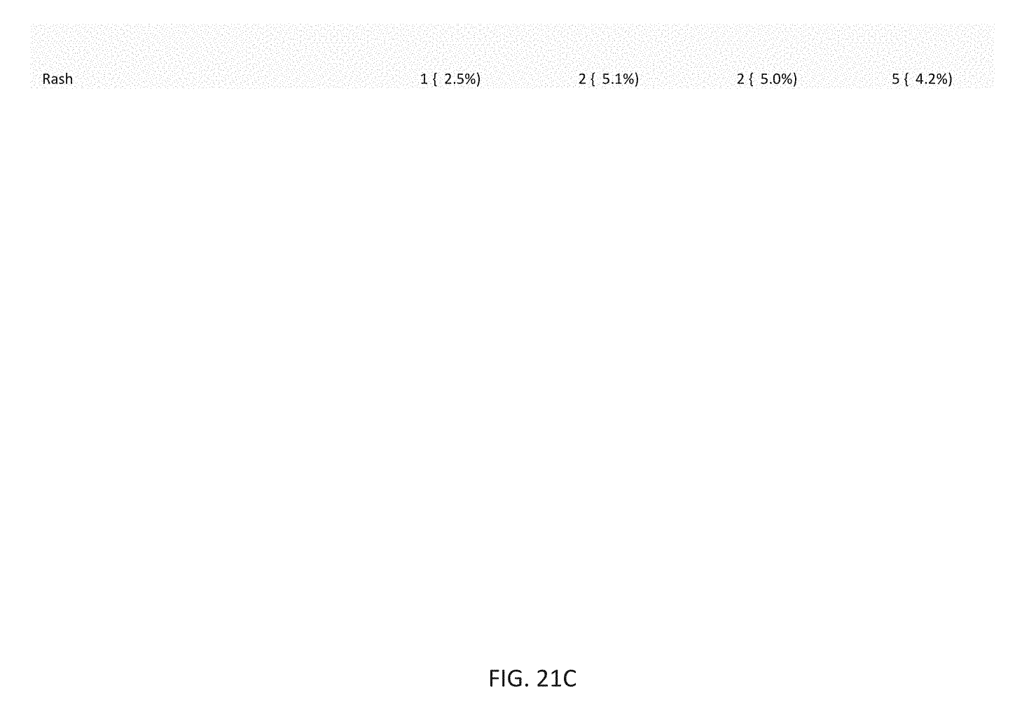

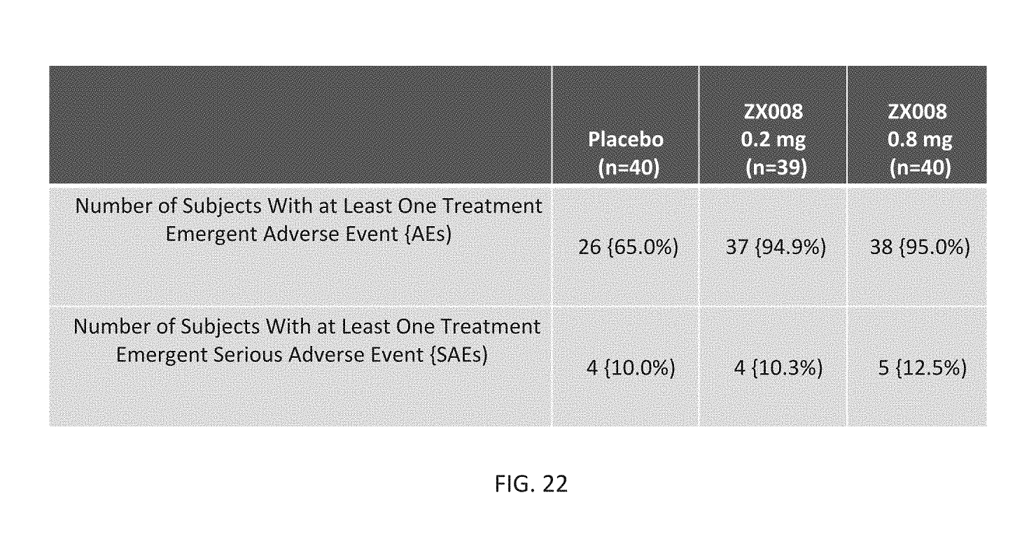

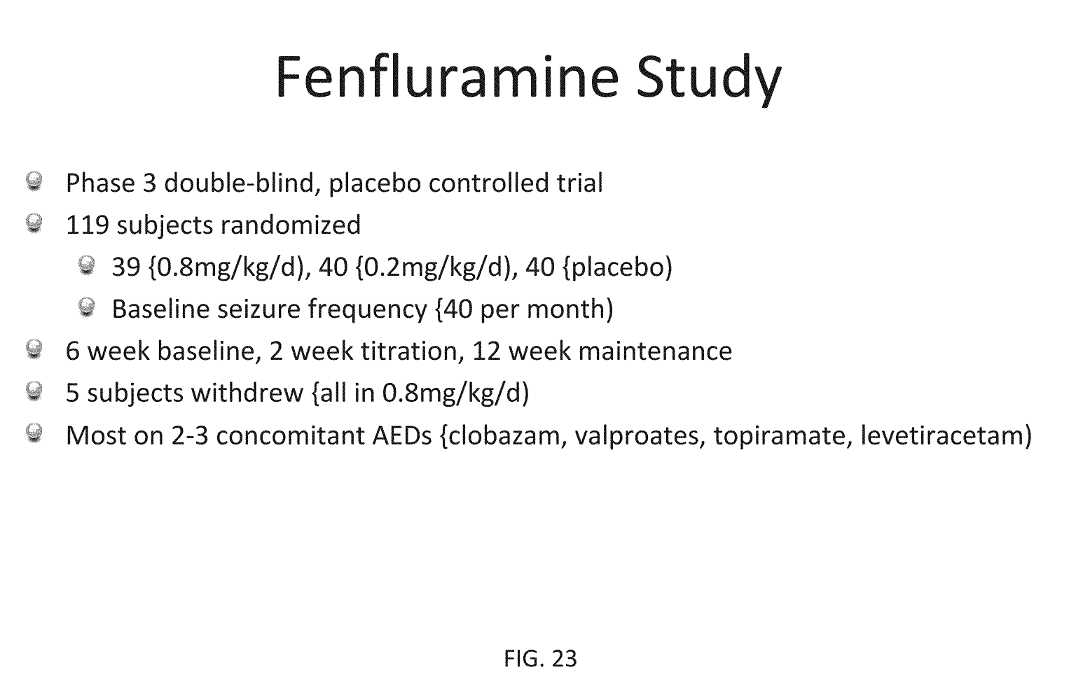

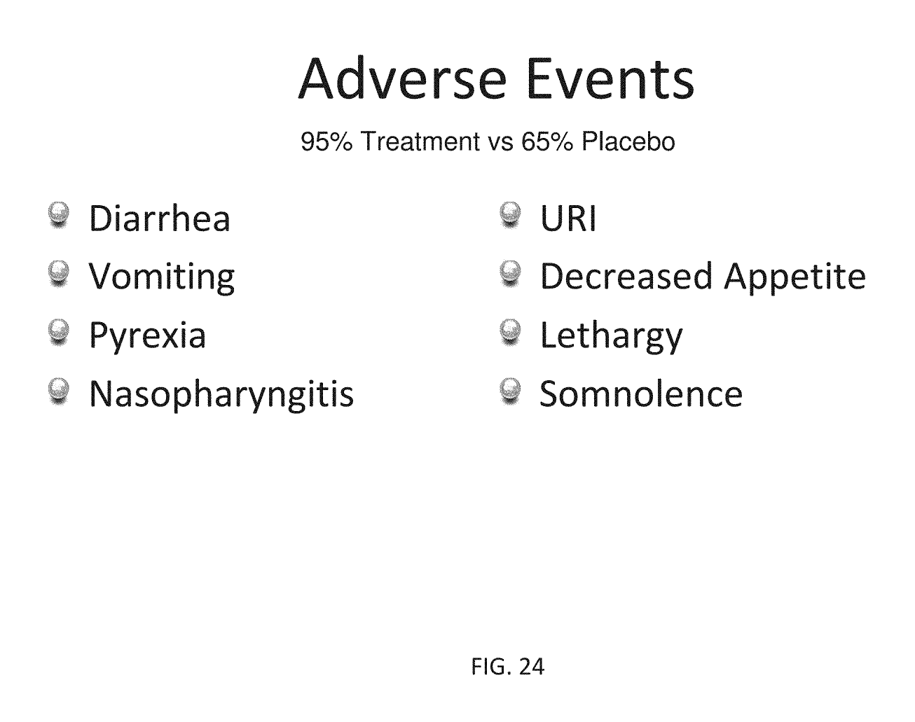

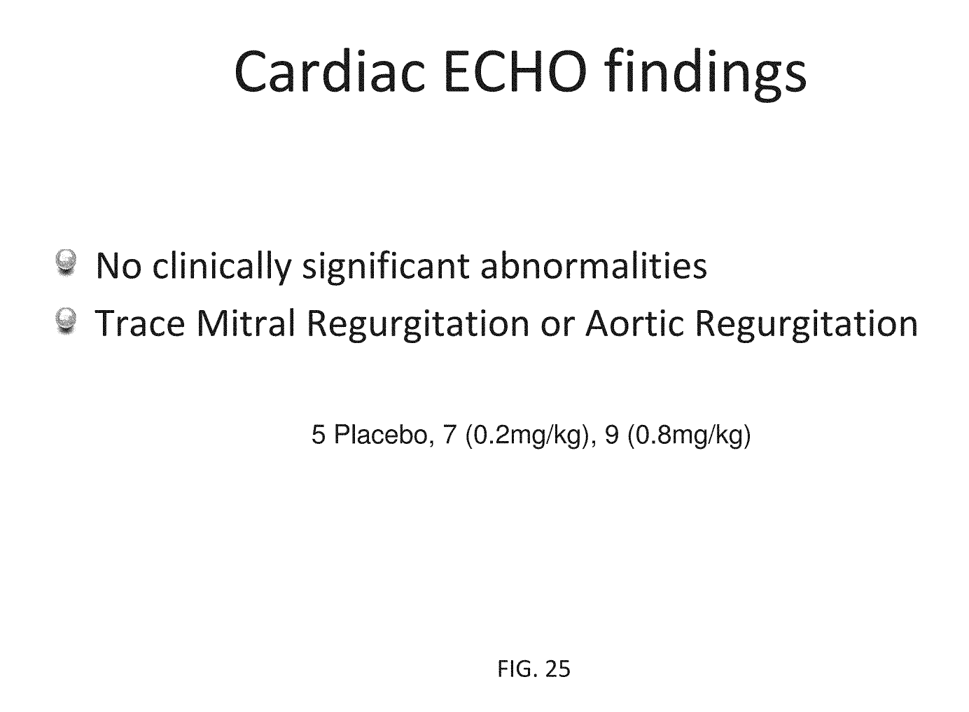

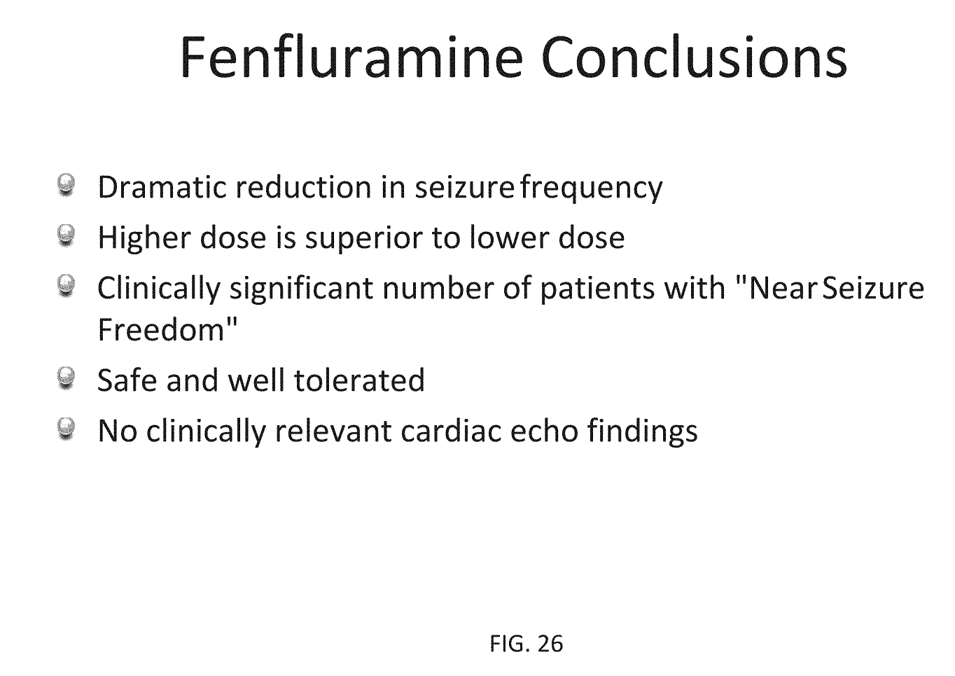

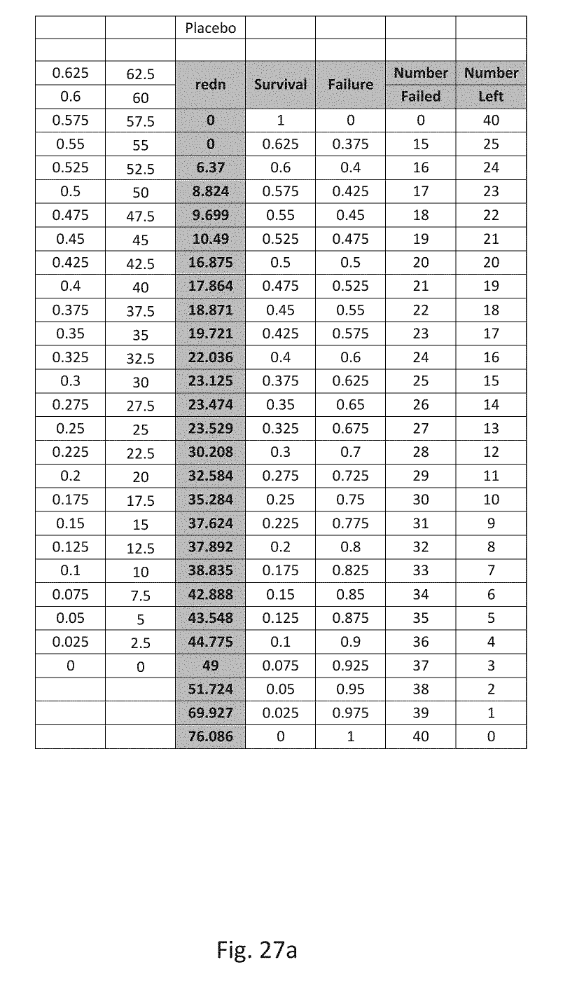

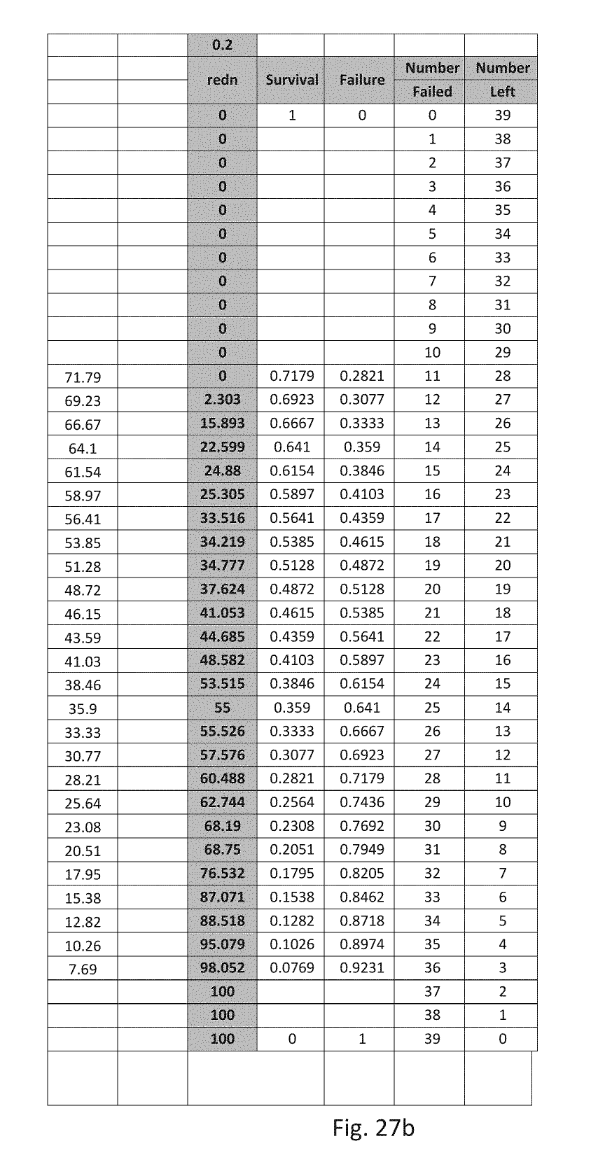

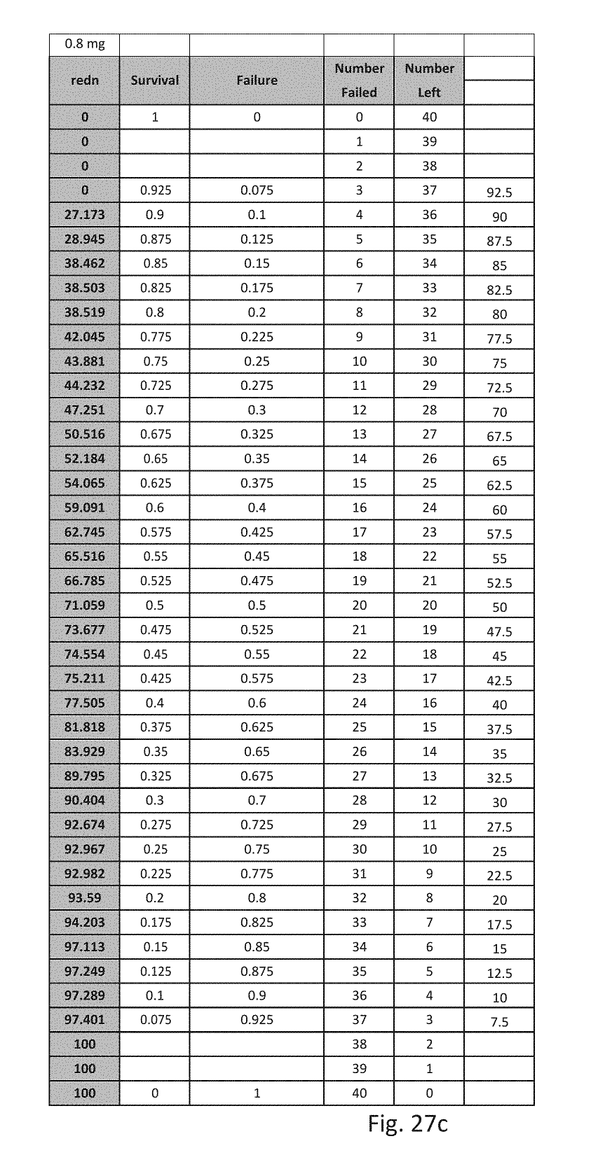

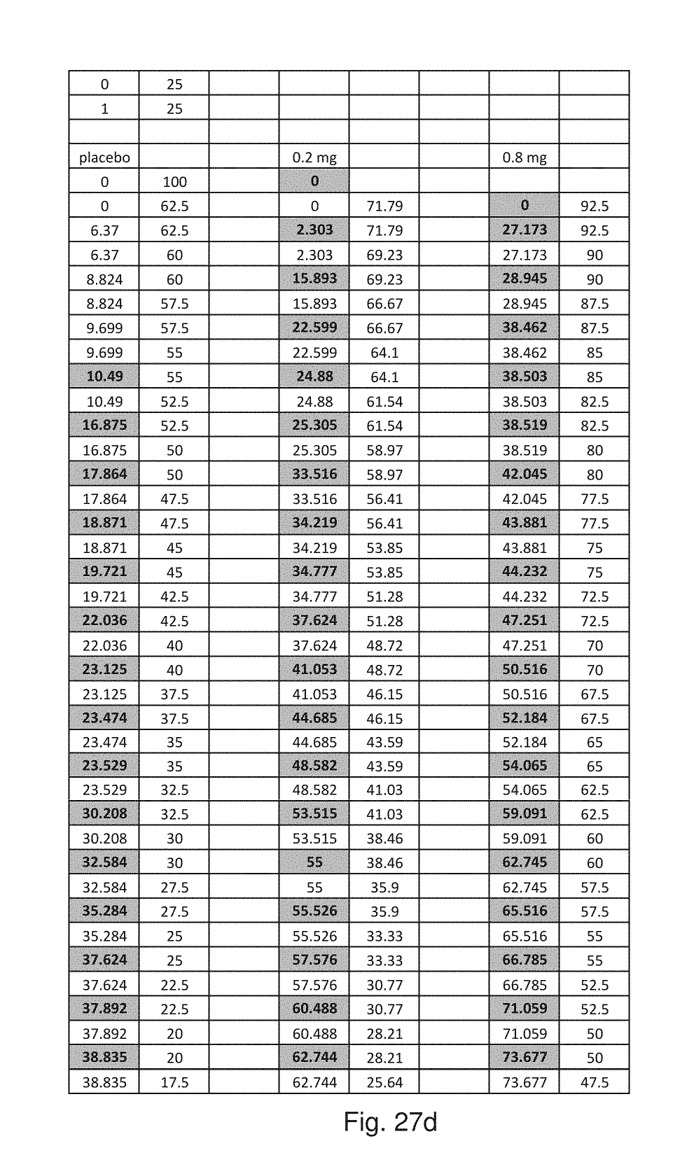

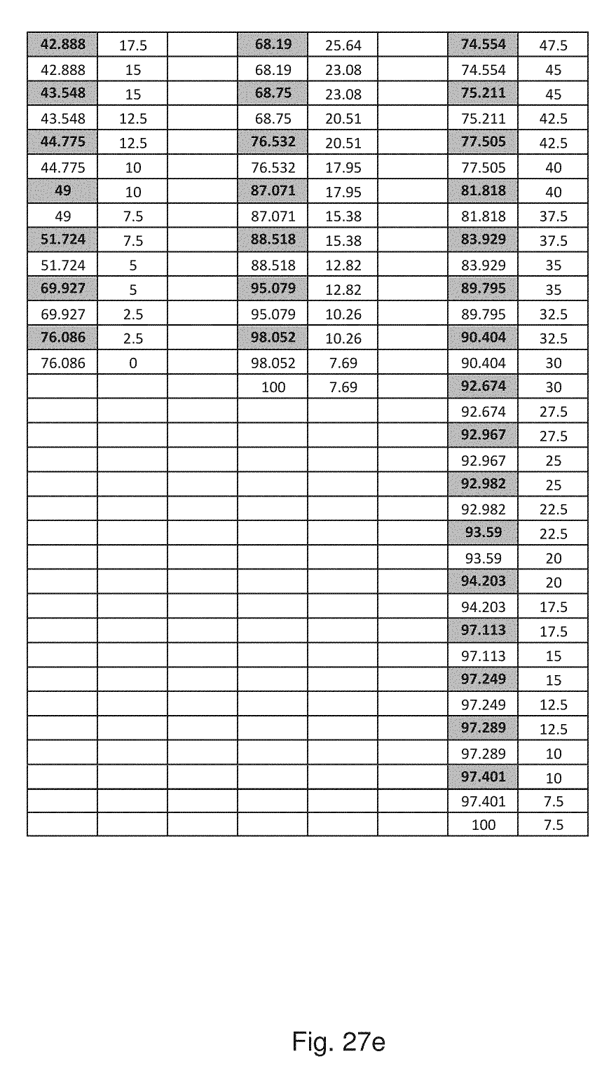

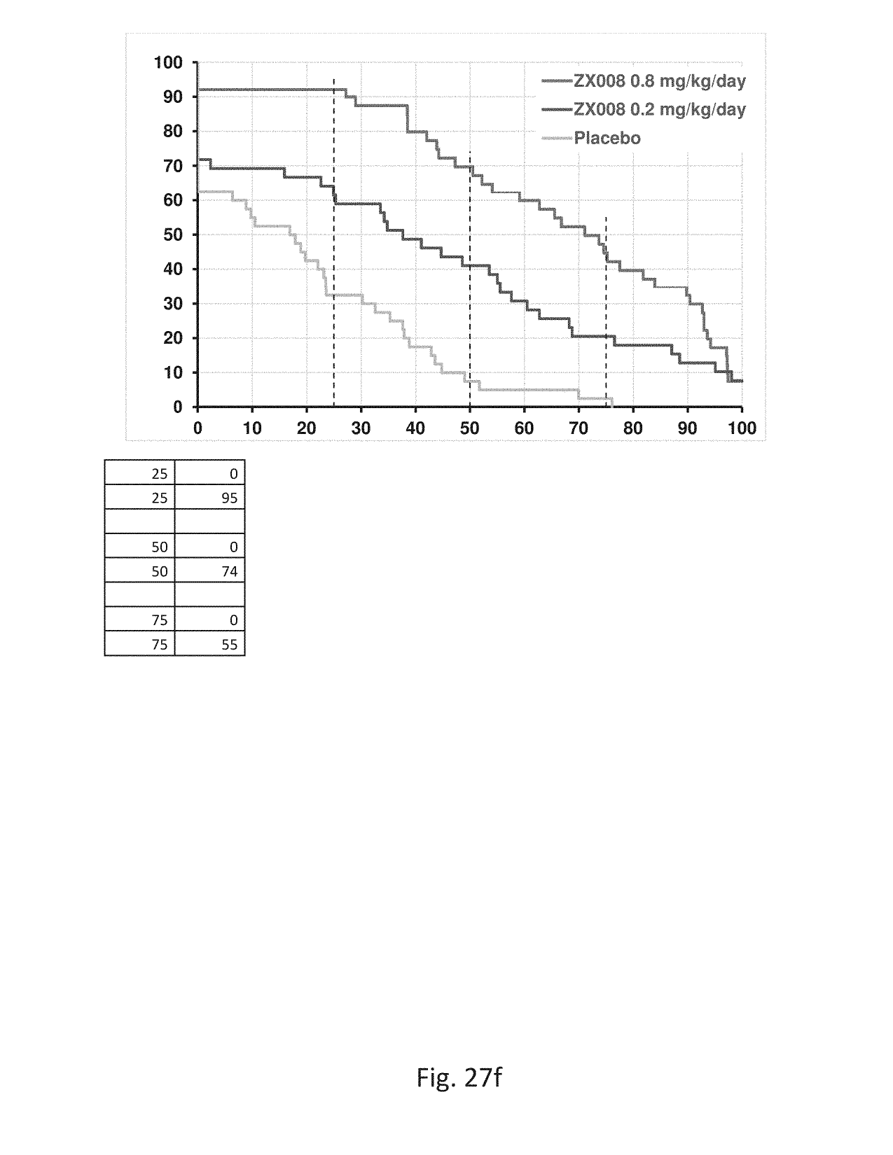

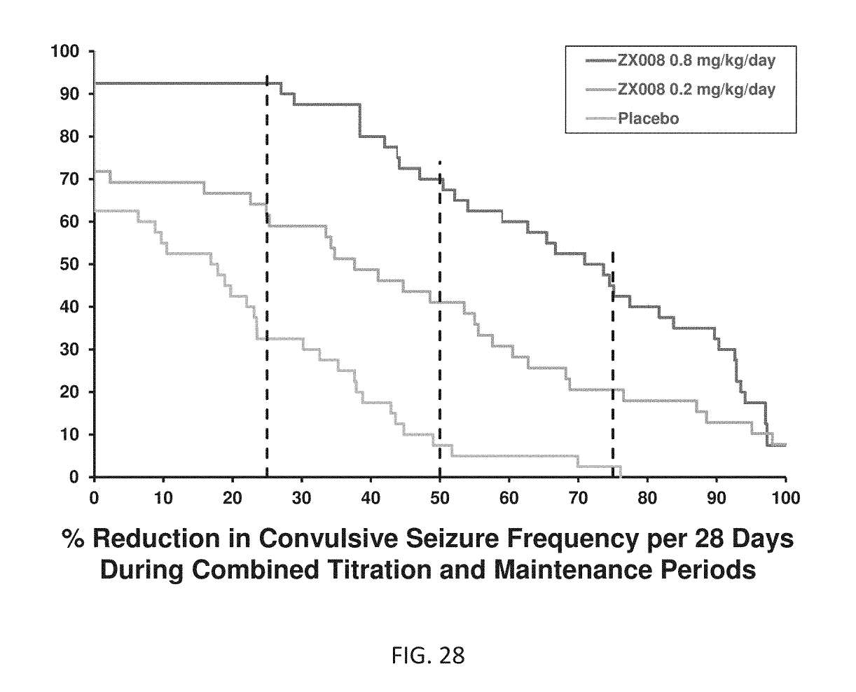

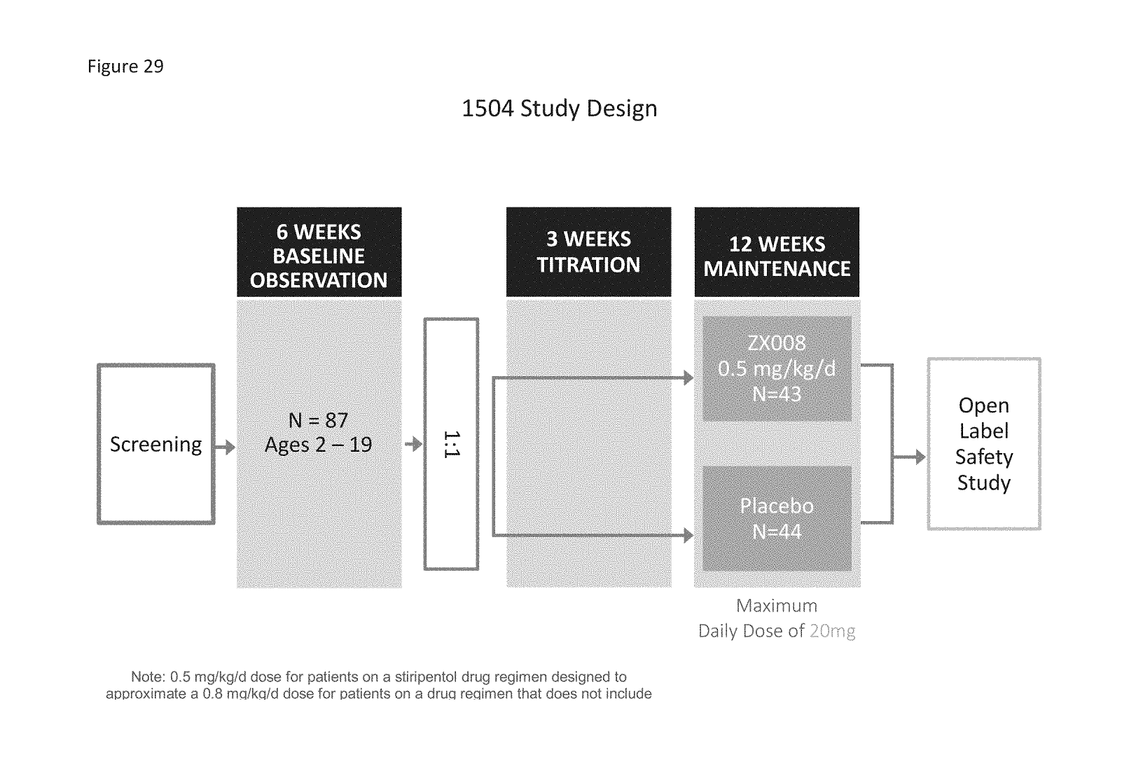

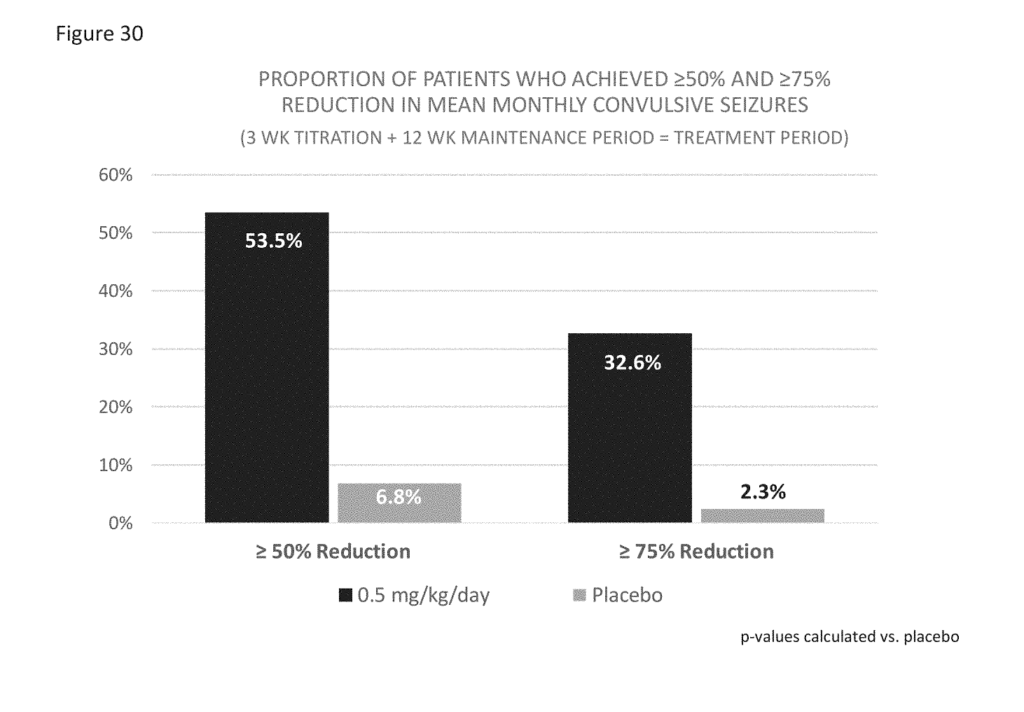

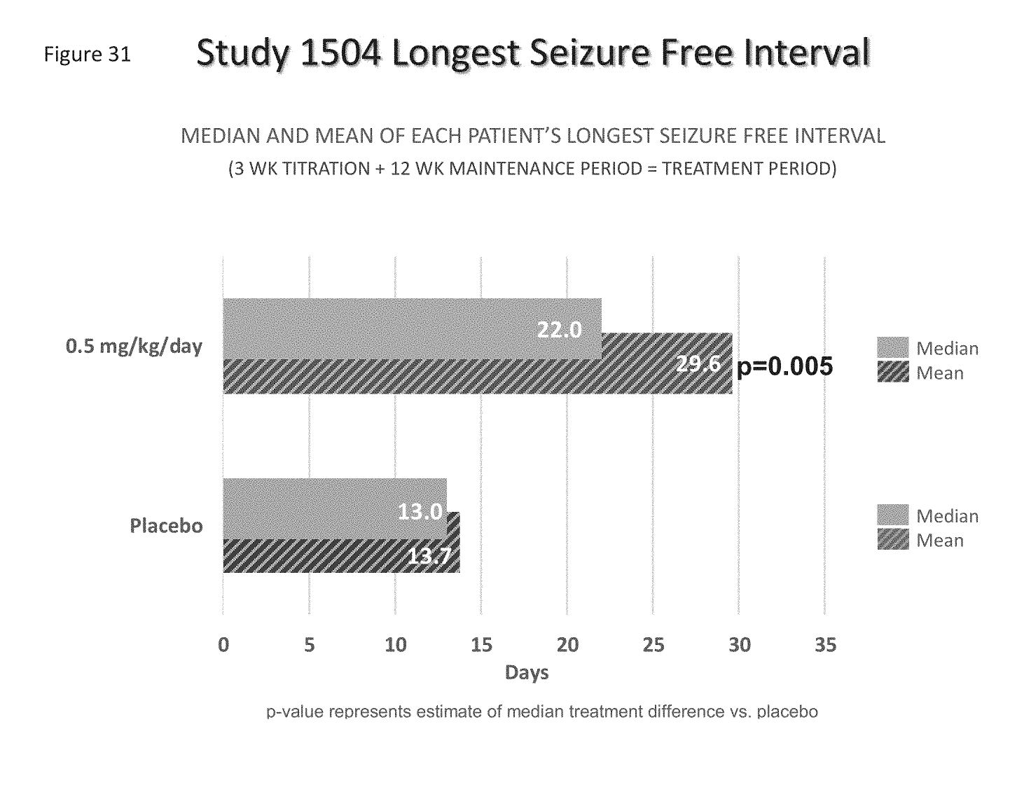

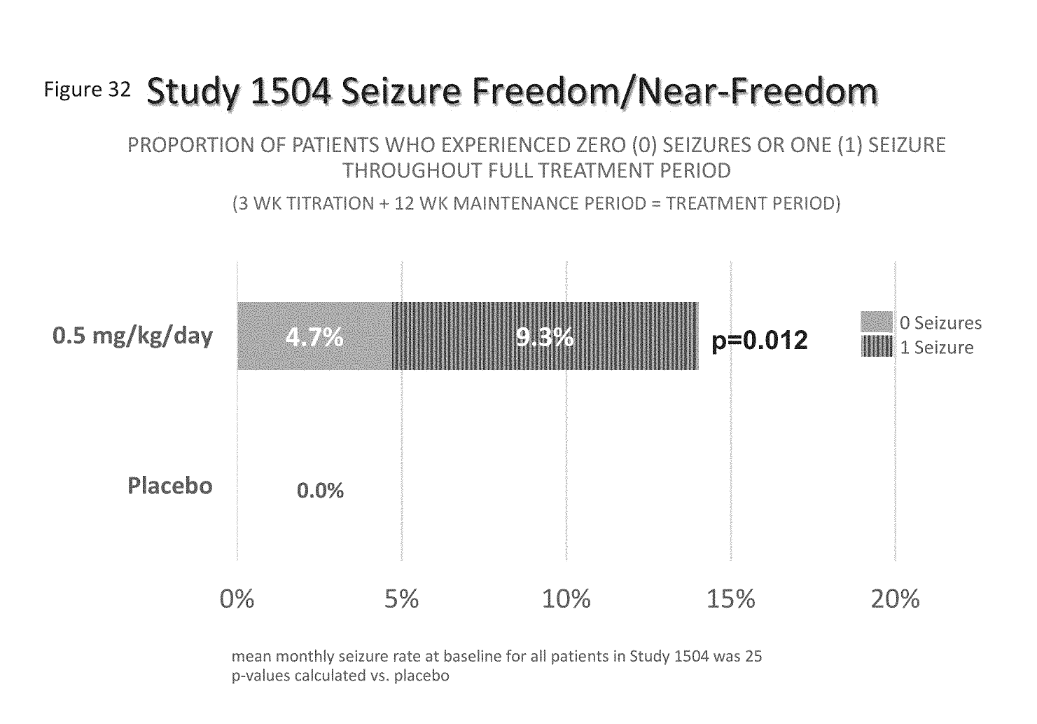

The primary efficacy measure was a comparison between liquid fenfluramine formulation 0.8 mg/kg/day and placebo of the change in monthly convulsive seizure frequency between during the 14-week treatment period and the 6-week baseline observation period. Patients taking liquid fenfluramine formulation 0.8 mg/kg/day achieved a 63.9% greater reduction in monthly convulsive seizures than patients taking placebo (p<0.001) (FIG. 6).

In addition, the study evaluated the monthly frequency of convulsive seizures during the 14-week treatment period compared with the 6-week baseline period. Patients taking liquid fenfluramine formulation 0.8 mg/kg/day achieved a median reduction in monthly convulsive seizures of 72% as compared with a 17% reduction on placebo.

A key secondary endpoint used the same technique as the primary analysis to compare liquid fenfluramine formulation 0.2 mg/kg/day and placebo. Patients taking liquid fenfluramine formulation 0.2 mg/kg/day achieved a 33.7% greater reduction in monthly convulsive seizures than patients taking placebo (p=0.019). Patients taking liquid fenfluramine formulation 0.2 mg/kg/day achieved a median reduction in monthly convulsive seizures of 38%. Collectively, these data suggest a dose-response relationship for liquid fenfluramine formulation in the treatment of convulsive seizures in Dravet syndrome.

Additional key secondary objectives of the study were to compare 0.8 mg/kg/day and 0.2 mg/kg/day liquid fenfluramine formulation (independently) with placebo in terms of (1) the proportion of patients who achieved ≥50% reductions in monthly convulsive seizures and the median of the longest convulsive seizure-free interval. These results are shown in the Table 2. The proportion of patients who achieved ≥75% seizure reductions, a secondary (non-key) efficacy measure, is also presented.

TABLE 2

| | liquid | liquid | |
|---|---|---|---|
| | fenfluramine | fenfluramine | |
| | formulation | formulation | |
| | 0.8 mg/kg/d | 0.2 mg/kg/d | Placebo |
| | (N = 40) | (N = 39) | (N = 40) |
| Patients with ≥50% | 70% | 41% | 8% |
| reduction in monthly | (p < 0.001) | (p = 0.001) | |
| convulsive seizures | | | |
| Patients with ≥75% | 45% | 21% | 3% |
| reduction in monthly | (p = 0.001) | (p = 0.001) | |
| convulsive seizures | | | |
| Longest seizure-free | 21 days | 14 days | 9 days |
| interval (median) | (p = 0.001) | (p = 0.011) | |

Table 3 presents more details of the statistical analysis on reduction in seizure frequency.

TABLE 3
Percentage Reduction in Convulsive
Seizure Frequency (mITT Population)

| T + M Period Distribution | | | |
|---|---|---|---|
| of Percentage Change from | | ZX008 | ZX008 |
| Baseline in convulsive | Placebo | 0.2 mg | 0.8 mg |
| seizure frequency | (N = 40) | (N = 39) | (N = 40) |
| ≥50%, n (%) | 3 (7.5) | 16 (41.0) | 28 (70.0) |
| Odds ratio (95% CI) | | 10.095 (2.480, | 29.098 (7.182, |
| | | 41.100) | 117.890) |
| p-value¹ | | 0.001 | <0.001 |

CI = confidence interval; mITT = modified intent-to-treat population.
¹Two separate logistic regression models that include a categorical response variable (achieved 50% percentage point reduction, yes or no) as a function of treatment group (Active or placebo), age group (<6 years, ≥6 years), and baseline convulsive seizure frequency were used.

Table 4 presents details of the statistical analysis on reduction in seizures per 28 days for 0.2 and 0.8 dosage groups vs placebo.

TABLE 4
Convulsive Seizure Frequency Per 28 Days: T +
M Period - Parametric Analysis (mITT Population)

| | | ZX008 | | ZX008 | |
|---|---|---|---|---|---|
| | Placebo | 0.2 mg | | 0.8 mg | |
| | (N = 40) | (N = 39) | | (N = 40) | |
| Baseline Summary Statistics | | | | | |
| Mean (SD) | 46.07 (40.704) | 47.17 | (99.636) | 32.95 | (31.480) |
| Median | 31.39 | 17.50 | | 21.17 | |
| Min, Max | 3.3, 147.3 | 4.8, 623.5 | | 4.9, 127.0 | |
| T + M Period Summary | | | | | |
| Statistics | | | | | |
| Mean (SD) | 40.56 (39.748) | 29.23 | (40.169) | 18.89 | (32.080) |
| Median | 26.03 | 14.33 | | 5.42 | |
| Min, Max | 3.2, 180.6 | 0.0, 202.11 | | 0.0, 169.9 | |
| T + M Period: Parametric Model | | | | | |
| Summary¹ Results on Log Scale¹ | | | | | |
| Least Squares Mean (SE) | 3.08 (0.127) | 2.67 | (0.130) | 2.07 | (0.126) |
| 95% CI for Least Squares Mean | 2.84, 3.33 | 2.42, 2.93 | | 1.82, 2.31 | |
| Difference from Placebo: | | | | | |
| Estimate of A − P (95% CI) [1] | | −0.41 | (−0.75, 0.07) | −1.02 | (−1.36, −0.68) |
| p-value for comparison with | | 0.019 | | <0.001 | |
| placebo² | | | | | |
| Original Scale | | | | | |
| Least Squares Mean3 | 21.8 | 14.5 | | 7.9 | |
| Comparison with placebo | | | | | |
| Estimate of Ratio (95% CI) [3] | | 0.66 | (0.47, 0.93) | 0.36 | (0.26, 0.51) |
| Difference from Placebo (95% CI)4 | | 33.74 | (7.06, 52.77) | 63.89 | (49.40, 74.22) |

CI = confidence interval; M = Maintenance; mITT = modified intent-to-treat population; SD = Standard Deviation; SE = Standard error; T = Titration.
¹Baseline, M, and T + M Period values were log transformed prior to analysis. To avoid taking log of 0, a value of 1 was added to the M, T + M Period values before log transformation.
²Results are based on an ANCOVA model with treatment group (3 levels) and age group (<6 years, ≥6 years) as factors, log baseline convulsive seizure frequency as a covariate and log convulsive seizure frequency (Titration + Maintenance, or Maintenance) period as response. The p-value was obtained from this ANCOVA model.
3The LS Mean and A − P difference and CI on the log scale were exponentiated.
4This is obtained from the LS Means on the log scale as follows: 100 * [1 − exp (LS mean active − LS mean placebo)].

Longest Interval between Convulsive Seizures

The median longest seizure-free interval was significantly longer in subjects treated with ZX008 0.8 mg/kg/day and ZX008 0.2 mg/kg/day compared with placebo. The median longest interval was 20.5 days for the 0.8 mg/kg/day group (p<0.001) and 14.0 days for the 0.2 mg/kg/day group (p=0.011), compared to 9.0 days for placebo. The mean (SD) longest seizure-free interval (days) was 27.5 (23.99) for the 0.8 mg/kg/day group, 22.0 (26.56) for the 0.2 mg/kg/day group, and 9.5 (5.38) for placebo. Table 5 presents additional analysis of seizure free intervals in the two treatment arms and placebo.

TABLE 5
Longest Interval (Days) between Convulsive
Seizures (mITT Population)

| | | ZX008 | ZX008 |
|---|---|---|---|
| | Placebo | 0.2 mg | 0.8 mg |
| | (N = 40) | (N = 39) | (N = 40) |
| Median | 9.00 | 14.00 | 20.50 |
| Mean (SD) | 9.53 (5.383) | 22.00 (26.559) | 27.53 (23.986) |
| Min | 2.0 | 3.0 | 2.0 |
| 25^{th} percentile | 5.50 | 7.00 | 7.00 |
| 75^{th} percentile | 11.00 | 21.00 | 39.00 |
| Max | 23.0 | 104.0 | 97.0 |
| Estimate of Median | | 5.00 | 11.50 |
| Treatment Difference | | | |
| 95% CI for Treatment | | 1.00, 9.00 | 5.00, 18.00 |
| Difference¹ | | | |
| p-value² | | 0.011 | <0.001 |

CI = confidence interval; Max = maximum; Min = minimum; mITT = modified intent-to-treat population; SD = standard deviation.
¹Based on Hodges-Lehmann estimator of treatment difference.
²From Wilcoxon rank sum test comparing active with placebo

### Number of Convulsive Seizure-Free Days

The mean number of convulsive seizure free days, defined as a day for which diary data are available and no convulsive seizures have been reported, was determined for each treatment group during the Baseline and T+M Periods. Using a parametric analysis, comparisons for the ZX008 groups to placebo were estimated from an ANCOVA model with treatment group (3 levels) and age group (<6 years, ≥6 _{y}ears) as factors, log baseline number of convulsive seizure free days as a covariate and log convulsive seizure free days (Titration+Maintenance, or Maintenance Period) as response. The mean (SD) number of convulsive seizure free days per 28 days during the Baseline Period was 12.6 (7.68), 15.7 (6.80), and 14.6 (7.59) for the placebo, 0.2 and 0.8 mg/kg/day groups, respectively. The mean (SD) number of convulsive seizure free days per 28 days during the T+M Period for the 0.8 mg/kg/day group was 20.4 (8.72) days which was statistically significantly greater (p=0.006) compared to 14.0 (7.12) days for the placebo group. The mean (SD) number of convulsive seizure free days for the 0.2 mg/kg/day group was 17.9 (8.04) days, which was numerically greater than placebo, but was not statistically significant (p=0.514). Similar results were obtained with a nonparametric analysis where the difference between the 0.8 mg/kg/day and placebo groups was statistically significant (p<0.001) while the difference between the 0.2 mg/kg/day and placebo groups was not (p=0.102).

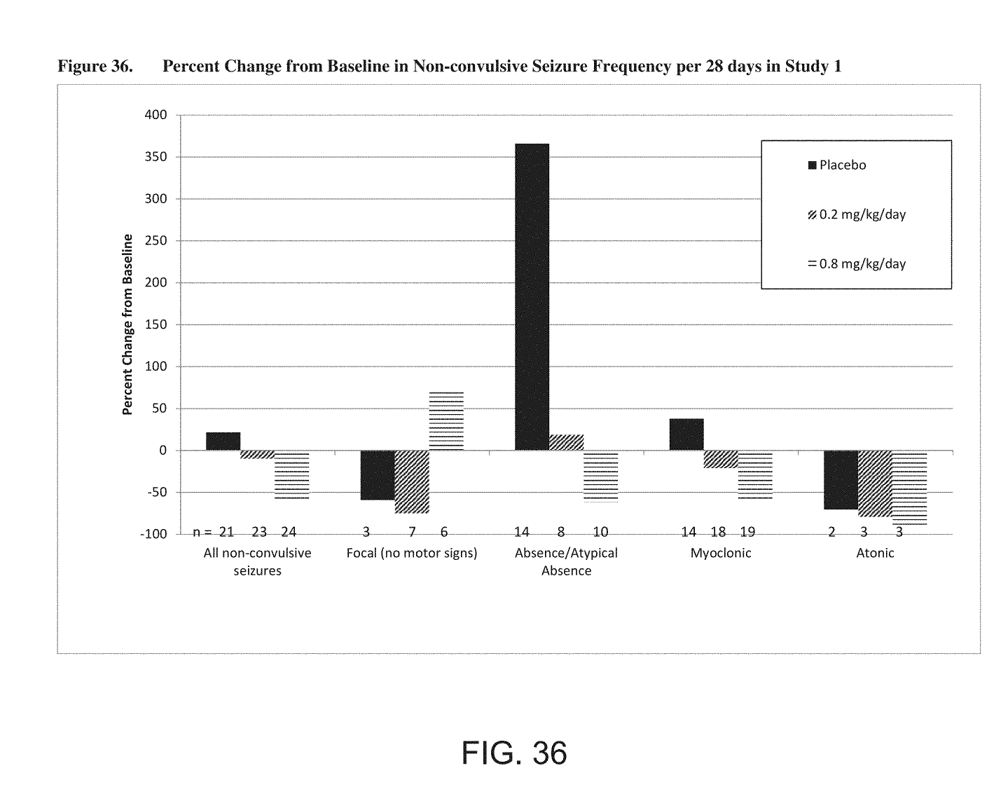

Fenfluramine's effect on nonconvulsive seizures was also tracked and analyzed. The mean (SD) number of non-convulsive seizures (all types) per 28 days at Baseline for the placebo, 0.2 and 0.8 mg/kg/day groups, respectively, was 67.8 (87.28), 193.6 (478.45), and 335.2 (758.58). At the end of the T+M Period the mean (SD) non-convulsive seizure frequency per 28 days for the 0.8 mg/kg/day group and placebo, respectively, was 127.9 (297.39) and 135.6 (463.26), which is a (%, [SD]) 59.1% (40.14) reduction for the 0.8 mg/kg/day group and a 21.5% (205.13) increase for placebo. The difference between placebo and the 0.8 mg/kg/day group was statistically significant (p=0.035). The change from Baseline for non-convulsive seizures (all types) per 28 days for the 0.2 mg/kg/day group was not statistically significant (p=0.735). At the end of the T+M Period the mean (SD) non-convulsive seizure frequency per 28 days for the 0.2 mg/kg/day group was 93.7 (219.69), which is a (%, [SD]) 9.7% (140.46) reduction from Baseline. The mean percent change from Baseline by non-convulsive seizure type is summarized graphically in FIG. 36.

The liquid fenfluramine formulation was orally administered with an oral syringe, and was generally well-tolerated in this study, with the most common adverse events consistent with the known safety profile of fenfluramine. The incidence of adverse events was higher in the treatment groups as compared to the placebo group, but the incidence of serious adverse events was similar in all three groups (Table 6). Five subjects in the 0.8 mg/kg/day group had an adverse event leading to study termination. Prospective cardiac safety monitoring throughout the study demonstrated no clinical or echocardiographic evidence of cardiac valvulopathy or pulmonary hypertension.

TABLE 6

| | ZX008 | ZX008 | |
|---|---|---|---|
| | 0.8 mg/kg/day | 0.2 g/kg/day | Placebo |
| | (n = 40) | (n = 39) | (n = 40) |
| Had serious adverse event | 5 (12.5%) | 4 (10.3%) | 4 (10.0%) |
| Study withdrawal all cause | 6 (15.0%) | 0.0 | 3 (7.5%) |
| Study withdrawal due to | 5 (12.5%) | 0.0 | 0.0 |
| adverse event | | | |

### Example 3

ZX008 (Fenfluramine HCL Oral Solution) in Dravet Syndrome: Effect on Convulsive Seizure Frequency in Patients Who Failed Treatment with Stiripentol Prior to Study 1

The effect of ZX008 on frequency of convulsive seizures (CSF) is assessed in a subset of Dravet syndrome (DS) subjects in a Phase 3 clinical trial (Study 1) who had previously been treated with stiripentol (58 subjects met the criteria for this analysis across both treatment arms and the placebo arm). For this analysis subjects who had discontinued stiripentol prior to study entry were defined as failures.

Stiripentol is approved in Europe, Australia, Canada and Japan and the USA for adjunctive treatment of patients with DS. A subgroup analysis of the effect of ZX008 on CSF in subjects who discontinued stiripentol prior to entry in Study 1 is presented.

Methods: Following a 6-week baseline period, subjects were randomized 1:1:1 to placebo (n=16), ZX008 0.2 mg/kg/day (n=20), or ZX008 0.8 mg/kg/day, maximum dose 30 mg/day (n=22) and treated for 14 weeks, including an initial 2-week titration period. Number and type of seizures were recorded daily in an electronic diary.

Results: A total of 58 subjects met the criteria for this analysis with a mean age 9.7 years (range, 2-18). ZX008 0.8 mg/kg/day showed a 60.8% reduction in mean monthly (28 days) CSF vs placebo (p=0.002). Seventy-three percent of subjects in the ZX008 0.8 mg/kg/day group achieved >50% reduction in CSF (p=0.006) and 50% achieved >75% reduction in CSF (p=0.036). Longest median seizure free intervals were 24.5 days (0.8 mg/kg/day, p=0.003), 18 days (0.2 mg/kg/day, p=0.012), and 9 days (placebo). Compared with placebo, ZX008 0.8 mg/kg/day-treated subjects were more likely to be rated much or very much improved by parents/caregivers (41% vs 6%, p=0.012) and investigators (64% vs 6%, p<0.001). ZX008 was generally well tolerated.

Conclusions: ZX008 provided robust improvement in CSF in subjects who had previously used stiripentol, an approved treatment for seizures in DS. ZX008 may represent an effective new treatment option for these patients with DS.

### Example 4

ZX008 (Fenfluramine HCL Oral Solution) in Dravet Syndrome: Effect on Convulsive Seizure Frequency in Patients Who Failed Treatment with Cannabidiol Prior to Study 1

The effect of ZX008 on frequency of convulsive seizures (CSF) is assessed in a subset of Dravet syndrome (DS) subjects in a Phase 3 clinical trial (Study 1) who had previously been treated with cannabidiol. For this analysis subjects who had discontinued cannabidiol prior to study entry were defined as failures.

Cannabidiol is being developed for adjunctive treatment of patients with DS and was recently given marketing approval by the US FDA, but is at this time, still not commercially available. A subgroup analysis of the effect of ZX008 on CSF in subjects who discontinued cannabidiol prior to entry in Study 1 (n=32) is presented.

Methods: Following a 6-week baseline period, subjects were randomized 1:1:1 to placebo (n=7), ZX008 0.2 mg/kg/day (n=11), or ZX008 0.8 mg/kg/day, maximum dose 30 mg/day (n=14) and treated for 14 weeks, including an initial 2-week titration period. Number and type of seizures were recorded daily in an electronic diary.

Results: A total of 32 subjects met the criteria for this analysis. ZX008 0.8 mg/kg/day showed a 67.8% reduction in mean monthly CSF. A total of 85.7% of subjects (n=12) in the ZX008 0.8 mg/kg/day group achieved >50% reduction in CSF and 64.3% of subjects (n=9) achieved >75% reduction in CSF. ZX008 0.2 mg/kg/day (n=11) showed a 22.6% reduction in mean monthly CSF. A total of 45.5% of subjects (n=5) in the ZX008 0.2 mg/kg/day group achieved >50% reduction in CSF and 9.1% of subjects (n=1) achieved >75% reduction in CSF.

Conclusions: ZX008 provided robust improvement in CSF in subjects who had previously used cannabidiol for seizures in DS. ZX008 may represent an effective new treatment option for these patients with DS.

### Example 5

A Randomized, Double-blind, Placebo-controlled Parallel Group Evaluation of the Efficacy, Safety, and Tolerability of ZX008 as Adjunctive Antiepileptic Therapy to Stiripentol Treatment in Children and Young Adults with Dravet Syndrome (Study 1504). Stiripentol (Diacomit®) is approved treatment for Dravet syndrome (DS) in Europe, Canada, Japan, the USA and Australia as an adjunctive therapy in patients with Dravet syndrome, and must be co-administered with clobazam with or without valproate.

Patient Inclusion and Dosing: A 6-week Baseline Period consisted of the establishment of initial eligibility during a screening visit followed by an observation period where subjects were assessed for baseline seizure activity based on recordings of daily seizure activity entered into a diary which established a baseline convulsive seizure frequency (CSF). Upon completion of the Baseline Period, subjects who qualified for the study were randomized (1:1) in a double-blind manner to receive ZX008 (at a dose of 0.5 mg/kg/day, maximum 20 mg/day or placebo.

Randomization was stratified by age group (≥2 to <6 years and ≥6 years) to ensure balance across treatment arms. Patients were titrated to their target dose over three weeks and then remained at that fixed dose for 12 weeks. Titration occurred in 3 steps starting with a 0.2 mg/kg/day dose of ZX008 (or placebo equivalent) on Study Days 1-7, increased to a dose of 0.4 mg/kg/day on Study Day 8-14, and then increased to a dose of 0.5 mg/kg/day on Study Days 15-21; the maximum daily dose at any point was 20 mg/day. The duration of the titration period was 21 days. Following titration subjects continued treatment at their randomly assigned dose of ZX008 0.5 mg/kg/day (maximum 20 mg/day) or placebo over a 12-week Maintenance Period.

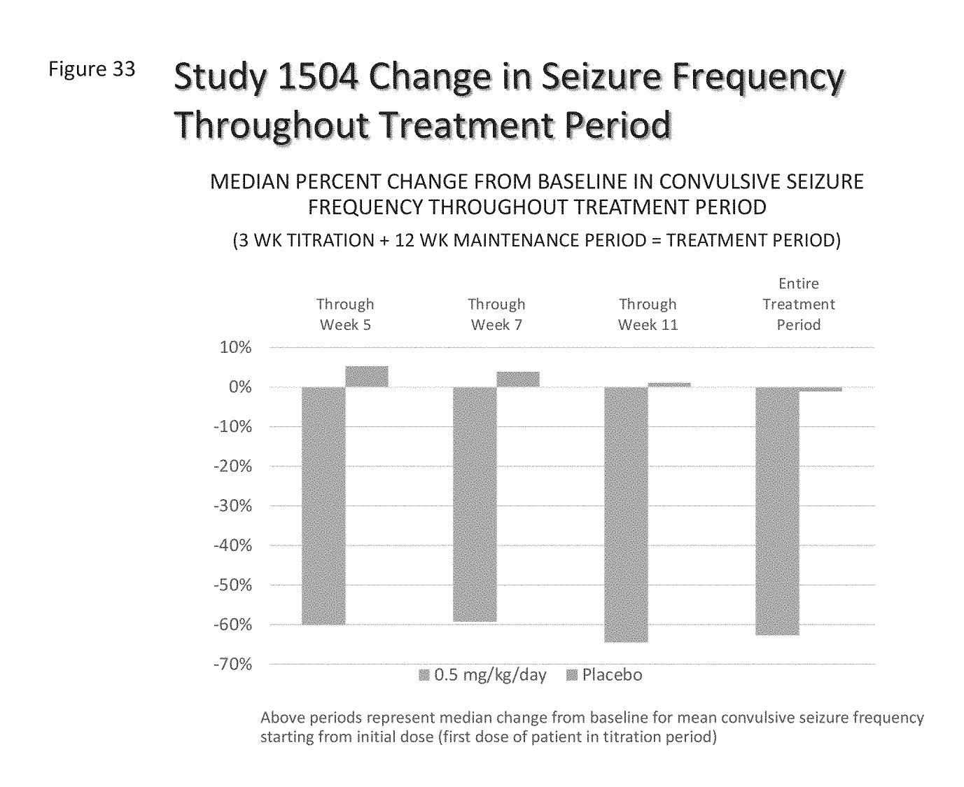

Eighty-seven patients were randomized into treatment and placebo arms, with a median age of 9 years (range, 2-19 years), across sites in Europe, the United States, and Canada. Following a six-week baseline observation period, which established a baseline CSF, patients were randomly assigned to one of two treatment groups in which ZX008 (n=43) or placebo (n=44) was added to their stable background regimen of stiripentol plus other antiepileptic drugs. The ZX008 dose of 0.5 mg/kg/day (20 mg maximum daily dose) in this study accounted for a drug-drug interaction between stiripentol and ZX008 and was designed to approximate the 0.8 mg/kg/day dose evaluated in Study 1 wherein patients background concomitant medications did not include a stiripentol regimen. The mean baseline CSF across the study groups was approximately 25 seizures per month. Additionally, the study did not find a diminishing effect of treatment with ZX008 over the 15-week treatment period (see FIG. 33), which is remarkable as fenfluramine is a serotonergic agent. Many neurologic drugs exhibit tachyphylaxis, a decreasing response to a drug after administration of a few doses, including anti-epileptics and selective serotonin reuptake inhibitors (SSRIs).

Eligible participants in this study were offered enrollment in a separate open-label extension trial. At the end of the Maintenance Period (or early discontinuation), subjects who did not enter the open-label extension underwent a tapering of ZX008 dosing over 14 days, after which they were off study medication. Subjects who enrolled in the separate open-label extension trial entered a 14-day transition period.

A follow-up ECHO, ECG and possibly a physical examination are to be performed 3-6 months after study drug discontinuation with early termination, or for those subjects who completed the study but do not enter the open-label extension trial.

Patients who received ZX008 achieved a 54.7% greater reduction in monthly convulsive seizures compared to placebo (p<0.001). The median reduction in monthly convulsive seizure frequency was 62.7% in the ZX008 group compared to 1.2% in placebo patients.

The primary efficacy endpoint of the 1504 study, the change in the monthly convulsive seizure frequency (MCSF) per 28 days between the Baseline and Titration and Maintenance (T +M) periods, was calculated from all available data collected during the Baseline or T+M Periods. This endpoint was analyzed using an analysis of covariance (ANCOVA) model with treatment group (ZX008 or placebo) and age group ((≥2 to <6 years, ≥6 years) as factors, and with baseline frequency as a covariate. The primary analysis compares the ZX008 group to the placebo group using a two-sided test at the a=0.05 level of significance.

ZX008 also demonstrated statistically significant improvements versus placebo in both key secondary measures, including patients with clinically meaningful reductions (>50%) in seizure frequency and longest seizure-free interval.

TABLE 7

| | ZX008 | |
|---|---|---|
| | 0.5 mg/kg/day | Placebo |
| | (N = 43) | (N = 44) |
| Patients with ≥50% reduction in | 53.5% | 6.8% |
| monthly convulsive seizures* | (p < 0.001) | |
| Patients with ≥75% reduction in | 32.6% | 2.3% |
| monthly convulsive seizures | (p = 0.004) | |
| Longest seizure-free interval | 22 days | 13 days |
| (median)* | (p < 0.005) | |

*Key secondary endpoints

Several topline findings on the secondary endpoints are given in Table 7. The first key secondary endpoint—the proportion of subjects who achieved a ≥50% reduction from baseline in convulsive seizure frequency—was derived directly from the primary endpoint. That is, the proportion of subjects in the ZX008 group who had a change in convulsive frequency of at least 50 percentage points were compared to the analogous proportion in the placebo group.

The comparison was made using a logistic regression model that incorporates the same factors and covariates as the ANCOVA used in the primary analysis. The analyses were performed using data collected over the T+M period.

The longest interval between convulsive seizures was be calculated for each subject over the entire T+M period. The ZX008 and placebo groups were compared using a Wilcoxon test.

Additional secondary efficacy objectives of the 1504 study were to demonstrate ZX008 superiority at 0.5 mg/kg dose to placebo on the following endpoints:
i. The proportion of subjects who achieve ≥25% reductions from baseline in convulsive seizure frequency.
ii. The change from baseline in non-convulsive seizure frequency.
iii. The change from baseline in convulsive+non-convulsive seizure frequency
iv. The incidence of rescue medication usage
v. The incidence of hospitalization to treat seizures
vi. The incidence of status epilepticus
vii. The change from baseline in health-related quality of life (HRQOL) measured using the Pediatric Quality of Life Inventory™ (PedsQL) Generic Core Scale
viii. The change from baseline in PedsQL Family Impact module score
ix. Change from baseline in subjects' quality of life measured using the Quality of Life in Childhood Epilepsy (QOLCE)
x. The change from baseline in the HRQOL of the parent/caregiver using the standardized measure of health status (EQ-5D-5L) scale
xi. The change from baseline on the impacts of the condition on parents and the family using the PedsQL family impact module.
xii. Clinical Global Impression—Improvement (CGI-I) rating, as assessed by the principal investigator
xiii. CGI-I rating, as assessed by the parent/caregiver

Many neurologic drugs exhibit tachyphylaxis, a decreasing response to a drug after administration of a few doses, including anti-epileptics and selective serotonin reuptake inhibitors (SSRIs). The study did not find a diminishing effect of treatment with ZX008 over the 15-week treatment period (see FIG. 33), which is remarkable as fenfluramine is a serotonergic agent.

Safety endpoints: To compare the safety and tolerability of ZX008 to placebo with regard to AEs, laboratory parameters, physical examination, neurological examination, vital signs (blood pressure, heart rate, temperature, and respiratory rate), ECGs, ECHOs, and body weight, and assessment of cognitive function.

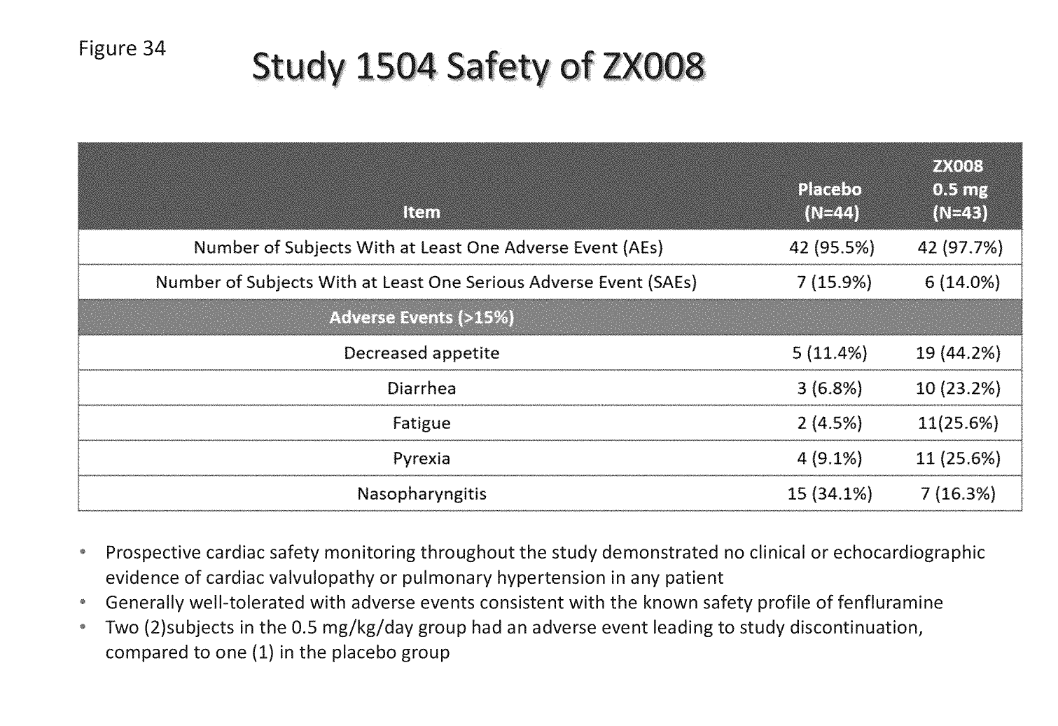

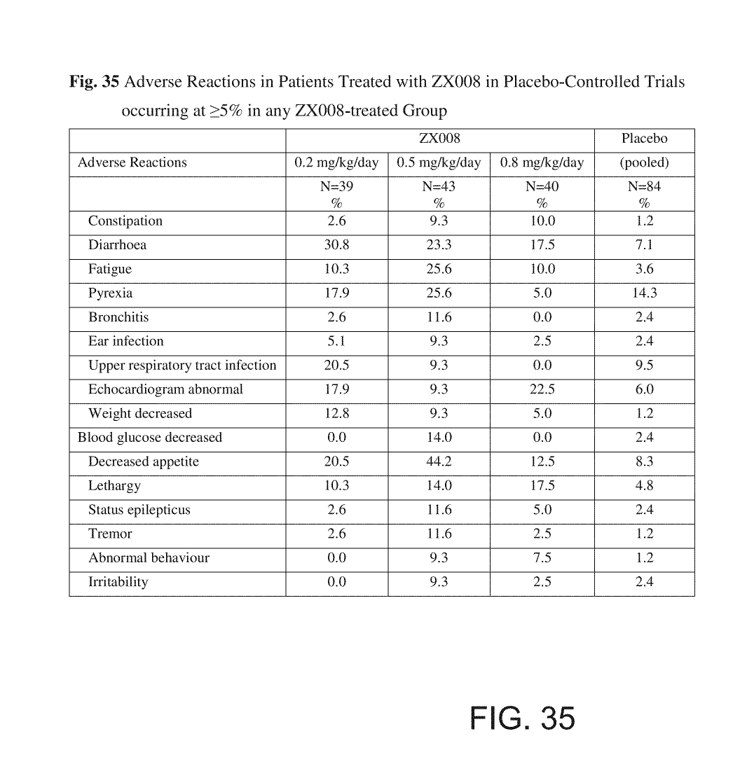

ZX008 was generally well-tolerated in this study, with the adverse events consistent with those observed in Study 1 and the known safety profile of fenfluramine. FIG. 34 shows adverse events in the 1504 study for both treatment and placebo groups and a listing of adverse effects occurring in greater than 15% of ZX008 treated patients. The incidence of treatment emergent adverse events was similar in both the treatment and placebo groups, with 97.7 percent (n=42) of patients receiving ZX008 experiencing at least one treatment emergent adverse event compared to 95.5 percent (n=42) of patients in the placebo group. The most common adverse events in the ZX008 group were decreased appetite, diarrhea, pyrexia, fatigue, and nasopharyngitis.

The incidence of serious adverse events was similar in both the treatment and placebo groups, with 14 percent (n=6) of patients in the ZX008 group experiencing at least one treatment emergent serious adverse event compared to 15.9 percent (n=7) of patients in the placebo group. Two patients in the ZX008 group had an adverse event leading to study discontinuation compared to one in the placebo group.

Prospective cardiac safety monitoring throughout the study demonstrated no clinical or echocardiographic evidence of cardiac valvular disease or pulmonary hypertension in any patient. These results confirm the observations from Study 1 which also reported no cardiac valvular disease or pulmonary hypertension. Furthermore, in the open-label safety extension study (Study 1503) there are approximately 300 patients currently enrolled, some of whom have been treated with ZX008 on a daily basis for over 2 years. In Study 1503, no safety signal of any clinically meaningful cardiovascular abnormality has been identified to date.

Conclusions: ZX008 in this study was successful in meeting its primary endpoint and both key secondary endpoints, demonstrating that ZX008, at a dose of 0.5 mg/kg/day (maximum 20 mg/day), is superior to placebo when added to a stiripentol regimen. The study findings are also consistent with results observed in Study 1, described in Examples 2, 3 and 4.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of treating a patient diagnosed with Dravet syndrome, comprising:
administering to the patient a therapeutically effective dose of fenfluramine or a pharmaceutically acceptable salt, base or acid thereof; and
repeating the administering over a period of days until the patient exhibits an increase from baseline in an average time between convulsive seizures of eight hours or more.

2. The method as claimed in claim 1, wherein the fenfluramine is the only active ingredient administered to the patient.

3. The method of claim 1, further comprising administering a co-therapeutic agent.

4. The method of claim 3, wherein the co-therapeutic agent is selected from the group consisting of, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt or base thereof.

5. The method of claim 4, wherein the co-therapeutic agent is a combination of stiripentol, valproate and clobazam.

6. The method of claim 1, wherein the co-therapeutic agent is cannabidiol.

7. The method of claim 1, wherein the administering is over a period of months, and the co-therapeutic agent is clobazam.

8. The method of claim 1, further comprising:
repeating the administering over a period of weeks until the patient exhibits an increase from baseline in an average time between convulsive seizures of one day or more.

9. The method of claim 1, further comprising:
repeating the administering over a period of weeks until the patient exhibits an increase from baseline in an average time between convulsive seizures of two days or more.

10. The method of claim 1, further comprising:
repeating the administering over a period of weeks until the patient exhibits an increase from baseline in an average time between convulsive seizures of one week or more.

11. The method of claim 1, further comprising:
repeating the administering over a period of weeks until the patient exhibits an increase from baseline in an average time between convulsive seizures of one month or more.

12. The method of claim 1, further comprising:
repeating the administering until the patient is seizure free for a period of ≥1 day.

13. The method of claim 1, further comprising:
repeating the administering until the patient is seizure free for a period of ≥9 days.

14. The method of claim 1, further comprising:
repeating the administering until the patient is seizure free for a period of ≥14 days.

15. The method of claim 1, further comprising:
repeating the administering until the patient is seizure free for a period of ≥21 days.

16. The method of claim 1, further comprising:
repeating the administering until the patient is seizure free for a period of ≥14 weeks.

17. The method of claim 1, further comprising:
repeating the administering until the patient is seizure free for a period of ≥6 months.

18. The method of claim 1, further comprising:
repeating the administering until the patient is seizure free for a period of ≥1 year.

19. The method of claim 1, further comprising:
repeating the administering until the patient is permanently seizure free.

20. A method of treating a patient diagnosed with Dravet syndrome, comprising:
administering to the patient a therapeutically effective dose of fenfluramine or a pharmaceutically acceptable salt, base or acid thereof in an amount of 0.2 mg/Kg/day or more, up to 30 mg/day;
administering a co-therapeutic agent; and
repeating the administering of the co-therapeutic agent and fenfluramine over a period of weeks until the patient exhibits an increase from baseline in average time between convulsive seizures of one week or more.
